(19) United States
(12) Patent Application Publication
(10) Pub. No.: US 2024/0194319 A1
(43) Pub. Date: Jun. 13, 2024

(54) DISPENSING DEVICE AND SERVER PROVIDING A COMBINATION OF DOSAGE FORMS OPTIMIZED FOR CURRENT HEALTH CONDITION OF USER AND OPERATING METHOD THEREOF

(71) Applicant: ALGOCARE CORPORATION, Seoul (KR)

(72) Inventors: Jiwon JOUNG, Seoul (KR); Pil Lip YOUN, Seoul (KR); Kwang Tae KIM, Seoul (KR)

(73) Assignee: ALGOCARE CORPORATION, Seoul (KR)

(21) Appl. No.: 18/555,185

(22) PCT Filed: Nov. 9, 2022
(86) PCT No.: PCT/KR2022/017499

§ 371 Date: Oct. 12, 2023

(30) Foreign Application Priority Data
Nov. 12, 2021 (KR) 10-2021-0155897
Dec. 9, 2021 (KR) 10-2021-0175808
Dec. 17, 2021 (KR) 10-2021-0182043
Jan. 28, 2022 (KR) 10-2022-0013172
Sep. 19, 2022 (KR) 10-2022-0118124

(51) Int. Cl. G16H 20/13; G16H 10/60; G16H 20/60

(52) U.S. Cl. CPC G16H 20/13; G16H 10/60; G16H 20/60

(57) ABSTRACT

According to an aspect of the present invention, there is provided an operating method of a server that processes dosage form composition information, the operating method including the steps of receiving, by a server, first current condition information about a user at a first time point, which is any one time point in a first period, calculating, by the server, first dosage form composition information by reflecting the first current condition information, transmitting, by the server, the first dosage form composition information to a device dispensing a dosage form, receiving, by the server, second current condition information about the user at a second time point, which is any one time point in a second period consecutive after the first period, calculating, by the server, second dosage form composition information by reflecting the second current condition information, and transmitting, by the server, the second dosage form composition information to the device.

### TECHNICAL FIELD

The present invention relates to a dispensing device and server providing a combination of dosage forms optimized for current health condition of a user, and a processing method thereof.

### BACKGROUND ART

As a nutrient market grows and needs of the user diversify, a personalized nutrient service that analyzes the health condition of the user, and combines and provides appropriate nutrients accordingly are being activated. This personalized nutrient service is performed by collecting health information of users through a medical examination-by-interview, etc., deriving a nutritional component determined necessary by analyzing collected health information of the user, and combining and providing the nutrients satisfying the nutritional component.

In the provision of nutrients, which is the third matter among the matters described above, conventionally, the nutrients have been operated in a manner in which they are combined and packaged in small portions at a factory. However, as such, in the case of customized nutrients produced in the factory, the collection and analysis of health information is not performed in real time, and thus there is a disadvantage that the health information of the user that changes on a daily basis cannot be immediately reflected. In addition, due to the minimum production quantity of the factory, there was no choice but to provide customized nutrients as a unit for a considerable period of time (e.g., one month).

### DISCLOSURE OF THE INVENTION

### Technical Problem

An object of the present invention is to provide a dispensing device and server that provide a combination of dosage forms optimized for a current health condition of a user, and a processing method thereof.

### Technical Solution

According to an example of the present invention, there is provided an operating method of a server that processes dosage form composition information, the operating method including the steps of receiving, by the server, first current condition information about a user at a first time point, which is any one time point in a first period; calculating, by the server, first dosage form composition information by reflecting the first current condition information, transmitting, by the server, the first dosage form composition information to a device dispensing a dosage form, receiving, by the server, second current condition information about the user at a second time point, which is any one time point in a second period consecutive after the first period, calculating, by the server, second dosage form composition information by reflecting the second current condition information, and transmitting, by the server, the second dosage form composition information to the device.

According to an example of the present invention, there is provided a server that processes dosage form composition information, the server including a memory, a processor that is connected to the memory and executes instructions included in the memory, and a server communication unit that transmits information calculated by the execution of the processor to a user terminal or a device for dispensing dosage forms, and receives information transmitted from the user terminal or the device, in which the processor controls the communication unit to receive first current condition information about a user at a first time point, which is any one time point in a first period, calculates first dosage form composition information by reflecting the first current condition information, controls the communication unit to transmit the first dosage form composition information to the device, controls the communication unit to receive second current condition information about the user at a second time point, which is any one time point in a second period consecutive after the first period, calculates second dosage form composition information by reflecting the second current condition information, and controls the communication unit to transmit the second formulation composition information to the device.

According to an example of the present invention, there is provided an operating method of a device that provides a combination of dosage forms, the operating method including the steps of obtaining, by the device, first current condition information from a user at a first time point, which is any one time point in a first period, dispensing, by the device, a combination of dosage forms corresponding to the first time point based on first dosage form composition information in which the first current condition information is reflected, obtaining, by the device, second current condition information from the user at a second time point, which is any one time point in a second period consecutive after the first period, and dispensing, by the device, a combination of dosage forms corresponding to the second time point based on second dosage form composition information in which the second current condition information is reflected.

According to an example of the present invention, there is provided a provides a combination of dosage forms, the device including a memory, a processor that is connected to the memory and executes instructions included in the memory, an input unit that receives information from the outside under the control of the processor, and a dosage form dispensing unit that combines and dispenses a plurality of dosage forms under the control of the processor, in which the processor controls the input unit to obtain first current condition information from a user at a first time point, controls the dosage form dispensing unit to dispense a combination of dosage forms corresponding to the first time point based on first dosage form composition information in which the first current condition information is reflected, controls the input unit to obtain second current condition information from the user at a second time point, and controls the dosage form dispensing unit to dispense a combination of dosage forms corresponding to the second time point based on second dosage form composition information in which the second current condition information is reflected.

According to an example of the present invention, there is provided an operating method of a server, the operating method including the steps of obtaining first base dose information set based on initial variable value information of a user, the first base dose information including a plurality of nutritional components and a taking-a-dose amount for each of the plurality of nutritional components and a taking-a-dose amount for each of the plurality of nutritional components being set according to the initial variable value information, extracting indication variable value information for the user from personal health information of the user, obtaining at least one important factor variable for the user among a plurality of factor variables according to the indication variable value information, calculating a detailed variation rate value according to the important factor variable, calculating a variation rate value for the indication variable value information by reflecting the the detailed variation rate value, and calculating second base dose information by reflecting the variation rate value in the first base dose information.

According to an example of the present invention, there is provided an operating method of a server for controlling a dispensing amount of a dispensing device, the operating method including the steps of obtaining first dose information for a user, the first dose information including first single-component dose information about a single-component cartridge that contains a single-component dosage form including a first nutritional component and first multi-component dose information about a multi-component cartridge that contains a multi-component dosage form including the first nutritional component and a second nutritional component, obtaining cartridge information about the dispensing device, the cartridge information including information about a cartridge mounted on the dispensing device, calculating second dose information based on the first dose information and the cartridge information, and providing a nutrient through the dispensing device using the calculated second dose information, in which, when the single-component cartridge and the multi-component cartridge are mounted on the dispensing device, the step of calculating of the second dose information includes the steps of calculating second multi-component dose information about the multi-component cartridge among the second dose information by using the first dose information and calculating second single-component dose information about the single-component cartridge among the second dose information by using the first dose information and the second multi-component dose information.

According to an example of the present invention, there is provided an operating method of a server for controlling a dispensing amount of a dispensing device, the operating method including the steps of obtaining first dose information for a user, the first dose information including first single-component dose information for a single-component cartridge that contains a single-component dosage form including a first nutritional component and first multi-component dose information for a multi-component cartridge that contains a multi-component dosage form including the first nutritional component and a second nutritional component, obtaining cartridge information about the dispensing device, the cartridge information including information about a cartridge being mounted on in the dispensing device, calculating second dose information based on the first dose information and the cartridge information, and providing a nutrient through the dispensing device using the calculated second dose information, in which, when the single-component cartridge and the multi-component cartridge are mounted on the dispensing device, in the step of calculating the second dose information, after calculating second multi-component dose information about the multi-component cartridge among the second dose information, second single-component dose information about the single-component cartridge among the second dose information is calculated.

According to an example of the present invention, there is provided an operating method of a server, the operating method including the steps of storing base dose information for a user, receiving first current condition information for the user, determining whether the first current condition information is received a first predetermined number of times or more during a predetermined period, providing a first body condition medical examination-by-interview corresponding to the first current condition information, receiving first medical examination-by-interview result information, which is a result of the user performing the first body condition medical examination-by-interview, and updating the base dose information based on the first medical examination-by-interview result information.

According to an example of the present invention, there is provided an operating method of a user terminal, the operating method including the steps of receiving, by the user terminal, first taking-a-dose information of a user at a first time point, determining, by the user terminal, whether or not the user takes a dose at a first taking-a-dose time point based on the first taking-a-dose information, if it is determined that the user has taken the dose at the first taking-a-dose time point, displaying, by the user terminal, a screen indicating taking-a-dose of the user, receiving, by the user terminal, second taking-a-dose information of the user at a second time point after the first time point, determining, by the user terminal, whether the user has taken or not taken a dose at a second taking-a-dose time point after the first taking-a-dose time point based on the second taking-a-dose information, and, if it is determined that the user has not taken the dose at the second taking-a-dose time point, outputting, by the user terminal, an alarm for encouraging the user to take the dose.

According to an example of the present invention, there is provided an operating method a user terminal, the operating method including the steps of obtaining, by the user terminal, alarm setting information from a user, transmitting, by the user terminal, the alarm setting information to a server, receiving, by the user terminal, alarm information according to the alarm setting information from the server, and outputting, by the user terminal, an alarm for encouraging the user to take a dose according to the alarm information.

According to an example of the present invention, there is provided an operating method of a user terminal, the operating method including the steps of obtaining, by the user terminal, alarm setting information including a taking-a-dose information including first alarm time, the alarm setting alarm setting information about a first taking-a-dose time point within a first period and second alarm setting information about a second taking-a-dose time point within the first period, transmitting, by the user terminal, the alarm setting information to a server, receiving, by the user terminal, first alarm information according to the first alarm setting information from the server, outputting, by the user terminal, a first alarm for encouraging the user to take a dose according to the first alarm information, receiving, by the user terminal, second alarm information according to the second alarm setting information from the server, and outputting, by the user terminal, a second alarm for encouraging the user to take the dose according to the second alarm information.

According to an example of the present invention, there is provided an operating method of a server, the operating method including the steps of obtaining, by the server, daily serving amount information for a user, the daily serving amount information including first serving amount information about a first cartridge and second serving amount information about a second cartridge, and a first nutrient and a second nutrient being stored in the first cartridge and second cartridge, respectively, receiving, by the server, alarm setting information including a taking-a-dose alarm time, the alarm setting information including first alarm setting information about a first divided time point within a first period and second alarm setting information about a second divided time point within the first period, calculating, by the server, a first division ratio for the first cartridge and a second division ratio for the second cartridge based on the alarm setting information, and providing, by the server, the nutrient to the user through a dispensing device according to the first division ratio and the second division ratio.

### Advantageous Effects

An embodiment according to the present invention can provide a combination of dosage forms optimized for the health condition of the user at each time point by reflecting current condition information about the health condition of the user at the time point when the combination of dosage forms is provided to the user.

### BRIEF DESCRIPTION OF THE DRAWINGS

FIG. 1 is a diagram illustrating an example of a network environment according to an embodiment of the present invention.

FIG. 2 is a diagram for explaining an example of a user terminal illustrated in FIG. 1.

FIG. 3 is a diagram for explaining an example of a configuration of a server illustrated in FIG. 1 and an example of processing dosage form composition information.

FIG. 4 is a diagram for explaining an example of a method of calculating base dose information in the present invention.

FIG. 5 is a diagram for explaining an example in which personal health variable value information is applied for each nutritional component when base dose information is calculated in the present invention.

FIG. 6 is a diagram for explaining an example in which current condition information is reflected in the base dose information in the present invention.

FIG. 7 is a diagram for explaining an example of a physical structure of a device illustrated in FIG. 1.

FIG. 8 is a diagram for explaining an example of a circuit configuration of the device illustrated in FIG. 7 and an example of a configuration of a device terminal.

FIG. 9 is a diagram for explaining an example of an operating method of the device illustrated in FIG. 7.

FIGS. 10A to 10C are diagrams for explaining a first embodiment of a method in which the server, the user terminal, and the device illustrated in FIG. 1 operate together.

FIGS. 11A and 11B are diagrams for explaining an example of outputting personalized result information by the user terminal in FIG. 10A.

FIGS. 12A and 12B are diagrams for explaining an example of a method for a device to obtain device input information at first and second time points of the first and second periods in FIG. 10B or FIG. 10C, respectively.

FIGS. 12C to 12D are diagrams for explaining an example of dispensing result information output by a device at first and second points of time in the first and second periods of FIG. 10B or FIG. 10C, respectively.

FIGS. 13A to 13D are diagrams for explaining example of taking-a-dose management information output by the user terminal corresponding to current condition information at the first and second time points of the first and second periods of FIG. 10B or FIG. 10C, respectively.

FIG. 14 is a table for explaining an example in which change amount information at a first time point is reflected.

FIG. 15 is a diagram for explaining an example in which change amount information of a second time point is reflected.

FIGS. 16A and 16B are diagrams for explaining a first modified example of the first embodiment described in FIGS. 10A to 10C.

FIGS. 17A and 17B are diagrams for explaining a second modified example of the first embodiment described in FIGS. 10A to 10C.

FIGS. 18A and 18B are views for explaining a third modified example of the first embodiment described in FIGS. 10A to 10C.

FIGS. 19A and 19B are diagrams for explaining a second embodiment of a method in which the service providing server, the user terminal, and the device illustrated in FIG. 1 operate.

FIGS. 20A and 20B are diagrams for explaining a first modified example of the second embodiment described in FIGS. 19A and 19B.

FIGS. 21A to 21C are diagrams for explaining a third embodiment of a method in which the service providing server, the user terminal, and the device illustrated in FIG. 1 operate.

FIG. 22A is a diagram for explaining an example of setting divided taking-a-dose through the user terminal and an example of displaying the number of times of divided taking-a-dose by the user terminal in FIG. 21A.

FIG. 22B is a diagram for explaining an example of a method of reflecting change amount information at a first and second divided time points in FIGS. 21A and 21B.

FIGS. 23A and 23B are diagrams for explaining a first modified example of a third embodiment described in FIGS. 21A to 21C.

FIGS. 24A to 24C are diagrams for explaining a fourth embodiment of a method in which the service providing server, the user terminal, and the device illustrated in FIG. 1 operate.

FIG. 25 is a diagram for explaining an example of a method in which a server updates base dose information.

FIG. 26 is a diagram for explaining an example of a method in which the server updates base dose information again.

FIG. 27A is a diagram for explaining an example in which the user terminal outputs body condition medical examination-by-interview result information in FIG. 25 or 26.

FIG. 27B is a diagram for explaining an example in which the user terminal displays cumulative taking-a-dose amount information, base dose information, average serving amount, minimum serving amount, and maximum serving amount.

FIG. 28 is a diagram for explaining an example of a method in which the user terminal outputs a taking-a-dose encouragement alarm.

FIG. 29 is a diagram for explaining an example in which the user terminal obtains alarm setting information.

FIG. 30 is a diagram for explaining an example in which a user terminal visually displays the taking-a-dose encouragement alarm.

FIG. 31 is a diagram for explaining an example in which a user terminal displays taking-a-dose information.

FIG. 32 is a diagram for explaining an example in which the user terminal obtains alarm modification information.

FIGS. 33A to 33C are diagrams for explaining an example of a method in which the server, the user terminal, and the device illustrated in FIG. 1 operate together for pre-dispensing.

FIG. 34 is a diagram for explaining in detail an operating method of a device for pre-dispensing in FIGS. 33A to 33C.

FIG. 35 is a diagram for explaining an example in which a user interface for pre-dispensing of a device is output when the device operates for pre-dispensing according to FIG. 34.

FIG. 36 is a diagram for explaining an example in which taking-a-dose management information is output to the user terminal for an N-th period and a pre-dispensing date in relation to pre-dispensing.

### MODE FOR CARRYING OUT THE INVENTION

The following content merely illustrates the principles of the present invention. Therefore, those skilled in the art can invent various devices, although not explicitly shown herein, implementing the principles of the present invention and fallen within the concept and scope of the present invention. Further, it should be understood that all conditional terms and examples listed in this specification, in principle, are expressly intended only for the purpose of enabling the concept of the present invention to be understood, and not to be limited to such specifically enumerated examples and conditions.

In addition, it should be understood that all detailed descriptions reciting specific embodiments, as well as principles, aspects and embodiments of the present invention, are intended to include structural and functional equivalents of these matters. In addition, it should be understood that such equivalents include not only currently known equivalents but also equivalents developed in the future, that is, all devices invented so as to perform the same function regardless of a structure.

Accordingly, for example, the block diagrams herein should be understood to represent conceptual views of exemplary circuits embodying the principles of the present invention. Similarly, it should be understood that all flow diagrams, state transition diagrams, pseudo code, etc. may be substantially represented on a computer readable medium and represent various processes performed by a computer or processor regardless of whether the computer or processor is explicitly illustrated.

Functionalities of various elements illustrated in the drawings including functional blocks represented by a processor or a concept similar thereto may be provided using dedicated hardware as well as hardware capable of executing software in relation to appropriate software. When provided by the processor, the functionalities may be provided by a single dedicated processor, a single shared processor, or a plurality of separate processors, some of which may be shared.

In addition, the explicit use of terms presented as the processor, control or s concepts should not be construed as exclusively citing hardware capable of executing software, and should be construed as implicitly including, without limitation, digital signal processor (DSP) hardware, a ROM, random access memory (RAM), and non-volatile memory for storing software. Other well-known and commonly used hardware may also be included.

In the claims of this specification, components expressed as means for performing the functions described in the detailed description are intended to include all methods of performing a function, including all types of software including, for example, a combination of circuit elements or firmware/microcode that performs the function, and the components are coupled with suitable circuitry for executing the software so as to perform the functions. Since the present invention as defined by these claims combines the functions provided by the various enumerated means and is combined in the manner required by the claims, any means capable of providing the functions should be construed as equivalent to those identified from this specification.

The object, features and advantages described above will become clearer through the following detailed description in relation to the accompanying drawings, and accordingly, those skilled in the art to which the present invention belongs can easily implement the technical idea of the present invention. In addition, in describing the present invention, if it is determined that a detailed description of a known technology related to the present invention may unnecessarily obscure the subject matter of the present invention, the detailed description thereof will be omitted.

Hereinafter, a preferred embodiment according to the present invention will be described in detail with reference to the accompanying drawings.

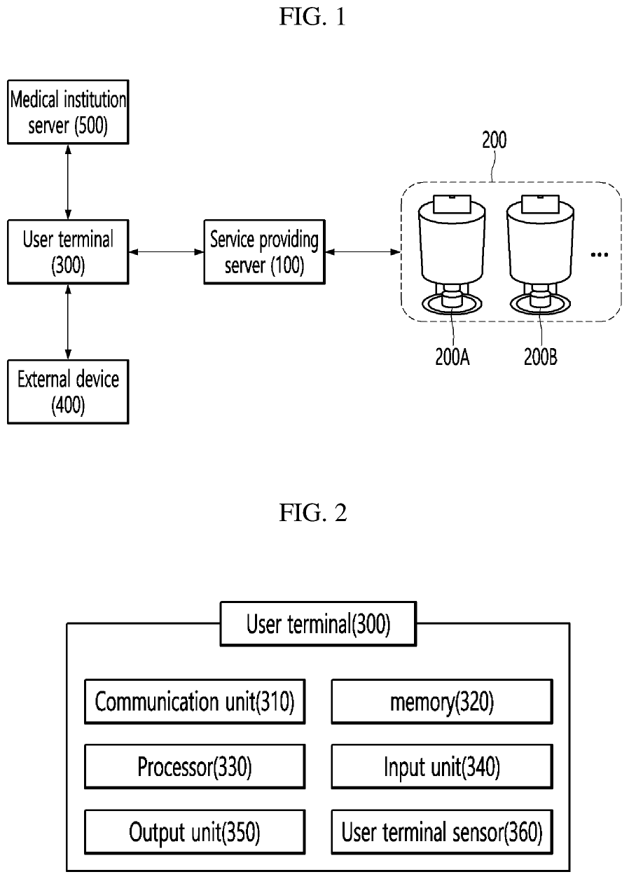

FIG. 1 is a diagram illustrating an example of a network environment according to an embodiment of the present invention.

As illustrated in FIG. 1, the network environment according to an embodiment of the present invention may include a service providing server 100, a dispensing device 200, a user terminal 300, an external device 400, and a medical institution server 500.

The network is not limited in communication method, and may include not only a communication method utilizing a communication network (e.g., a mobile communication network, wired Internet, wireless Internet, and broadcasting network) that the network may include, but also short-distance wireless communication.

As an example, the network may be implemented as all types of wired/wireless networks such as a local area network (LAN), a wide area network (WAN), a value added network (VAN), a personal area network (PAN), a mobile radio communication network or satellite communication network.

The service providing server 100 illustrated in FIG. 1 may be connected to the user terminal 300 and the device 200 through a network.

The service providing server 100 may receive information provided from the medical institution server 500 and the external device 400 through the user terminal 300. However, the present invention is not limited thereto, and information provided from the medical institution server 500 and the external device 400 may also be directly received without going through the user terminal 300.

The service providing server 100 (hereinafter referred to as “server”) may be implemented as a computer device that communicates with the user terminal 300 and the device 200 through a network to provide commands, codes, files, contents, services, etc.

For example, the server 100 of the present invention may provide information about a combination of dosage forms optimized for changes in the health condition of the user to the device 200 when the user receives the dosage form through the device 200.

To this end, the server 100 may calculate base dose information based on personal health information of the user, receive information about the current condition related to the health of the user (hereinafter referred to as ‘current condition information’) during a period to which a time point, at which the user receives the dosage form, belongs, reflects the information to the base volume information in real time, and calculate information about the combination of dosage forms to be provided to the user through the device 200.

For example, the server 100 may calculate the base dose information for each cartridge. As another example, the server 100 may calculate the base dose information for each nutritional component.

The period to which the time point, at which the user receives the dosage form, belongs may mean a date or a predetermined length of time to which the time point, at which the user receives the dosage form, belongs. For example, if the user receives the dosage form at 7 am on December 24th, the period to which the time point, at which the dosage form is provided, belongs may mean the length of time from 0:00 to 24:00 on December 24th or December 24th.

Therefore, the server 100 of the present invention may provide information about the combination of dosage forms optimized for the health condition of the user, and provide information about the combination of dosage forms optimized for the health condition of the user based on the current time by reflecting current condition information of the user in real time.

A dosage form means a substance in various forms, e.g., solid form, powder form, or liquid form, containing various nutritional components that can be absorbed by the body of the user. The dosage form is, e.g., processed into health functional food, pharmaceuticals, general food, etc., and taken for the purpose of improving normal function and health of the human body, and may have at least one form among various forms such as tablet, capsule, pill, powder, granule, jelly, or liquid. Hereinafter, a case in which the dosage form is in solid form will be described as an example.

When the dosage form includes nutrients, the dosage form may include, for example, nutritional components such as vitamin B, vitamin C, Omega-3, magnesium, multi-mineral, milk thistle, red ginseng, etc.

Nutrients may include one or more nutritional components.

For example, one nutrient may include one nutritional component. As a more specific example, a magnesium nutrient may include magnesium as a nutritional component. As another more specific example, an iron nutrient may include iron as a nutritional component. As another more specific example, a lactic acid bacteria nutrient may include lactic acid bacteria as a nutritional component.

As another example, one nutrient may include a plurality of nutritional components. As a more specific example, a multi-mineral nutrient may include magnesium and iron as nutritional components.

The type of nutrient may vary depending on the nutritional components contained in the nutrient. For example, a magnesium nutrient containing magnesium and an iron nutrient containing iron may be different types of nutrients. As another example, a magnesium nutrient containing magnesium and a multi-mineral nutrient containing magnesium and iron may be different types of nutrients.

In this specification, the fact that a nutrient contains only specific nutritional components means that it contains only specific nutritional components among various nutritional components, and does not mean that additives such as excipients, disintegrants, lubricants, anti-caking agents, coating agents, and pigments for forming a solid dosage form are excluded.

If the dosage form contains pharmaceuticals or prescription drugs, the dosage form may include pharmaceuticals or prescription drugs effective for chronic diseases or, e.g., aspirin or metformin, which has a preventive effect on cardiovascular disease or heart attack.

Personal health information of the user may include information about a chronic or chronic physical or mental disease, symptom, or condition of the user. The personal health information of the user may be extracted from at least one of identification information, medical examination-by-interview result information, or medical information about the user received by the server 100 from the user terminal 300 or the device 200.

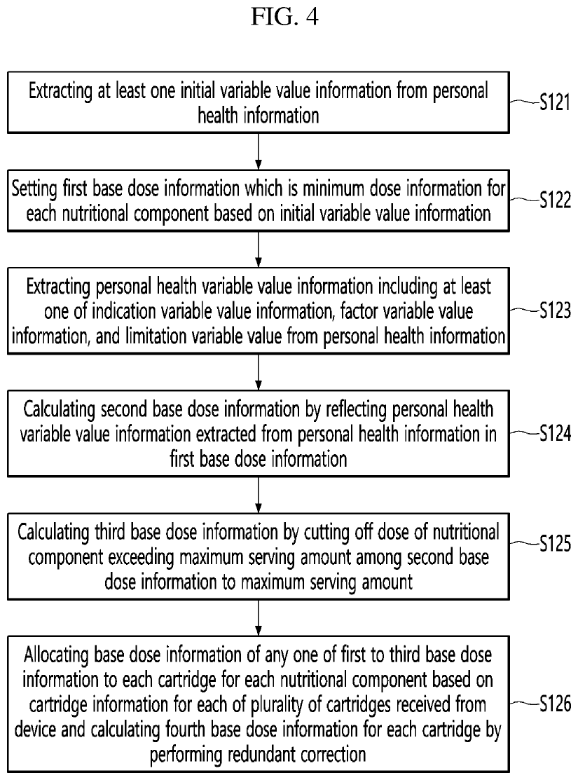

The base dose information may be information in which personal health variable value information extracted from the personal health information of the user is reflected for each nutritional component or each cartridge. That is, the base dose information may be personalized base dose information for each nutritional component or each cartridge optimized for the personal health information of the user. A method of calculating such base dose information will be described later with reference to FIG. 4 or subsequent drawings.

The current condition information of the user may include information about a mental or physical health condition related to a body condition of the user, or living condition, which is temporarily generated in relation to the health of the user for a specific period of time.

Specifically, the current condition information may include at least one of (1) information about a mental state or symptom of the user (e.g. depression, tension, anxiety, fatigue, stress, etc.) or (2) information about the physical state or symptom of the user (e.g. menstrual cramp, premenstrual syndrome (PMS), diarrhea, constipation, colds, stomatitis, migraines, etc.), and further include information about a behavior (e.g. overtime, examination, before exercise, after exercise, etc.), mood (e.g., in a good mood, not in a good mood, etc.), and severity of symptoms (e.g., severe menstrual cramp, mild menstrual cramp, etc.) of the user, that may affect health for a temporary period of time.

In addition, as the current condition information, any information can be used as long as it is information that can detect a temporary change in the body condition of the user. For example, biometric information collected from biometric signal of the user may be further included as the current condition information. For example, information about the amount of exercise (e.g., running distance, walking distance, etc.), blood pressure information, heart rate information, electrocardiogram information, etc. of the user acquired by various sensors may also be included in the current condition information, and voice information input from the user and image information generated by photographing the user may also be included in the current condition information.

The current condition information of the user may be collected by the device 200 or the user terminal 300, and the server 100 may receive the current condition information from the device 200 or the user terminal 300.

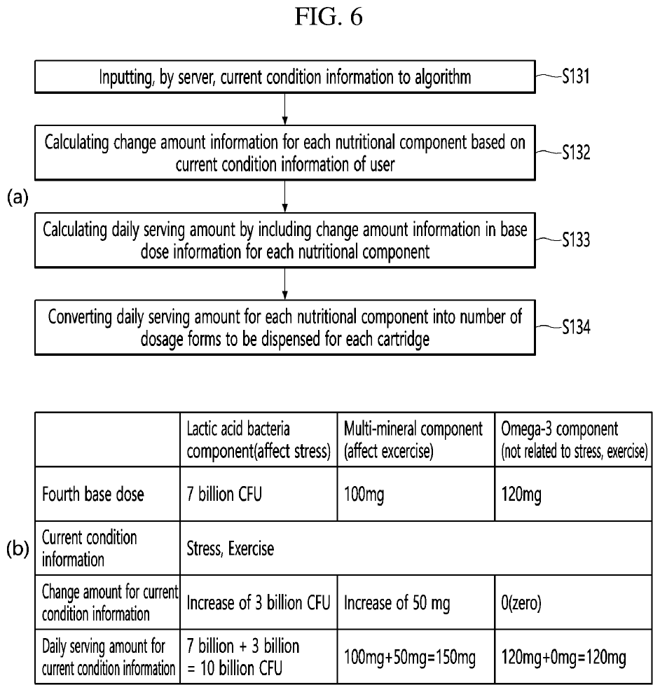

In the present invention, at the time point at which the user receives the dosage form from the device 200, the device 200 may provide a combination of dosage forms optimized for real-time body condition change of the user as daily serving amount information by reflecting the current condition information of the user in the base volume information in real time. As described above, a method of reflecting the current condition information in the base dose information will be described later with reference to FIG. 6 or later.

Here, the daily serving amount information is a serving amount for each nutritional component or each cartridge provided by the device 200 for a predetermined period of time (e.g., 1 day or 24 hours), and may be calculated by reflecting the current condition information of the user in the base dose information.

Here, the current condition information of the user can be used to calculate the daily serving amount of the period to which a time point, at which the current condition information is acquired, belongs, and may not be used to calculate the daily serving amount of the period to which the time point, at which the current condition information is acquired, does not belong.

For example, when first current condition information of the user is collected in a first period and second current condition information of the user is collected in a second period, the first current condition information may be used to calculate the daily serving amount of the first period, and the second current condition information may be used to calculate the daily serving amount of the second period.

The server 100 of the present invention may also receive, from the device 200, dispensing amount information about a dosage form that is dispensed through the device 200 or taken by the user. The server 100 may continuously monitor dispensing amount information from at least one device 200 and provide the information to the user terminal 300. For example, the server 100 may receive dispensing amount information from a first device 200A or receive dispensing amount information from a plurality of devices including the first and second devices 200A and 200B and transmit the dispensing amount information to one user terminal 300.

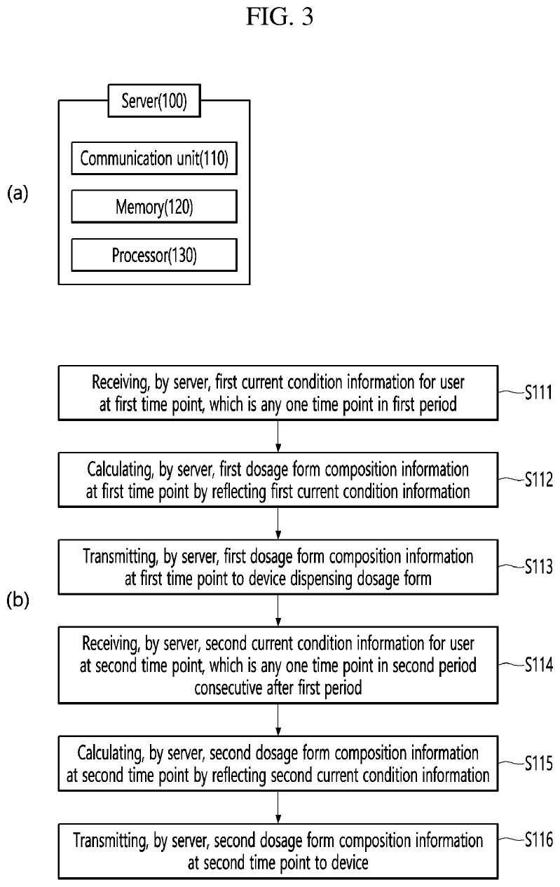

A configuration and basic function of the server 100 will be described in more detail with reference to FIGS. 3 to 6.

The dispensing device 200 (hereinafter referred to as ‘device’) may perform a function of dispensing a plurality of dosage forms to the user. Specifically, the device 200 may be interlocked with the server 100 through a network, obtain various information including current condition information, data or commands from the user through user interaction, and provide a plurality of dosage forms combined according to dosage form composition information reflecting the current condition information to the user.

To this end, the device 200 may store a plurality of dosage forms containing various nutritional components therein in advance.

One or more cartridges may be mounted on the dispensing device 300. A plurality of cartridges may be mounted on the dispensing device 300.

One cartridge may contain one type of nutrient. For example, the magnesium cartridge may contain a magnesium nutrient. As another example, a multi-mineral cartridge may contain a multi-mineral nutrient.

At the time point at which the user receives the dosage form through the device 200, the device 200 may dispense the number of combined dosage forms according to dosage form composition information based on current condition information. Accordingly, the device 200 may provide a dosage form optimized for the health of the user to the user according to real-time body condition change of the user.

In addition, the number of dosage forms provided to the user may be counted for each type of dosage form, and dispensing amount information about the number of dosage forms may be generated and provided to the server 100.

A detailed description of the device 200 will be described later with reference subsequent drawings.

The user terminal 300 may be a fixed terminal implemented as a computer device or a mobile terminal. As an example, the user terminal 300 may include a smart phone, a mobile phone, a tablet PC, a computer, a laptop computer, personal digital assistants (PDA), etc.

The user terminal 300 may be used by a user. The term “user” may refer to the user of the user terminal 300 or a user of an account registered for a service provided by the server 100. Therefore, the meaning that the server 100 transmits or provides certain information to the user may mean that certain information is transmitted to the user terminal 300 of the account registered in the server 100 and provided to the user.

The user terminal 300 may be interlocked with the server 100 through a network using a wireless or wired communication method, receive at least one of the base dose information, current condition information, and daily serving amount information described above as taking-a-dose management information from the server 100 and provide the information to the user.

After receiving medical examination-by-interview inquiry information from the server 100 and performing medical examination-by-interview to the user, the user terminal 300 transmit medical examination-by-interview result information, which is information about the result value of the medical examination-by-interview, to the server 100.

The user terminal 300 may request medical information of the user from the medical institution server 500 through an authentication means and obtain the medical information from the medical institution server 500 in response to the request, and the user terminal 300 may provide the received medical information to the server 100. The medical information may include, for example, disease history information, health checkup information, pharmaceuticals-in-use information, etc. of the user.

When the user terminal 300 obtains current condition information from the user through user interaction, the user terminal 300 may transmit the obtained current condition information to the server 100. The user interaction may mean that the user inputs information reflecting the selection or intention of the user to the user terminal 300 by manipulating an input means. The input means may include at least one of a touch screen a keyboard, a mouse, physical buttons, a camera, and a microphone provided in the user terminal 300 or connected to the user terminal 300.

The user terminal 300 may transmit sensing information of the user detected by a sensor (e.g., momentum information obtained by an acceleration sensor and a position sensor provided in the user terminal 300, etc.) provided therein or sensing information of the user detected from the external device 400 (e.g., blood pressure information obtained by a sensor interlocked with the user terminal 300, oxygen saturation information, etc.) interlocked with the user terminal 300 to the server 100 as the current condition information.

To this end, the user terminal 300 may include an application for performing the functions described above, and may be interlocked with the service providing server 100 through the application.

The configuration of the user terminal 300 will be described in more detail with reference to FIG. 2 and subsequent drawings.

The external device 400 may be a device that is interlocked with the user terminal 300, collects health-related information about the user of the user terminal 300, and transmits the collected health-related information to the server 100 through the user terminal 300.

For example, the external device 400 may include a device capable of collecting and detecting biometric signals from the user, and may include, for example, various wearable healthcare devices such as a smart watch, smart glasses, and a smart bracelet equipped with a sensor for collecting and detecting biometric signals.

The external device 400 may collect various health-related sensing information such as heart rate information, electrocardiogram information, blood pressure information, and blood sugar information corresponding to user information as the current condition information. The current condition information collected by the external device 400 may be transmitted to the server 100 through the user terminal 300.

The medical institution server 500 may be a database server of a hospital, medical center, a health examination institution, or other national medical institutions, etc. that receives and provides medical information requested from the user terminal 300.

When the medical institution server 500 receives a request for medical information of a user from the user terminal 300, the medical institution server 500 may provide the medical information to the user terminal 300 or provide the medical information to the server 100.

In one embodiment of the present invention including the server 100, the device 200 and the user terminal 300, base dose information for each nutritional component or each cartridge may be calculated based on personal health information of the user, the dosage form composition information may be calculated by reflecting the current condition information of the user in the base volume information in real time at the time point at which the user receives the dosage form, and the type and dose of a dosage form optimized according to dosage form composition information may be provided to the user.

Therefore, the present invention may optimize and provide a plurality of dosage forms and doses to the user according to the personal health information of the user, and may provide a dosage form more optimized for the current body condition of the user by reflecting current real-time current condition information of the user in the personal health information.

Hereinafter, the present invention will be described in more detail.

FIG. 2 is a diagram for explaining an example of the user terminal 300 illustrated in FIG. 1.

As illustrated in FIG. 2, the user terminal 300 includes a communication unit 310, a memory 320, a processor 330, an input unit 340, and an output unit 350, and may further include a user terminal sensor 360.

The communication unit 310 provided in the user terminal 300 may transmit information to the server 100, the external device 400 or the medical institution server 500, or receive information generated by the server 100, the external device 400 or the medical institution server 500.

The memory 320 may store an application for the user terminal 300 to perform a service provided by the server 100, or may store various commands or execution programs necessary for performing services provided by the server 100 and various health-related information related to the user.

The processor 330 may execute an application stored in the memory 320 to perform a medical examination for the user, collect health-related information of the user (e.g., personal health information or current condition information, etc.), or control the medical examination-by-interview result information or the health-related information of the user to be transmitted to the server 100 through the communication unit 310.

In addition, the processor 330 may control the health-related information of the user (e.g., personalized result information or taking-a-dose management information) provided from the server 100 to be output as information recognizable by the user.

The input unit 340 may collect or obtain various information input from the user through user interaction according to the execution of the application. The input unit 340 may include, for example, a keyboard, a mouse, a camera, and a microphone.

The output unit 350 may output the health-related information of the user provided from the server 100 according to the execution of the application. The output unit 350 may include, for example, a speaker and a display module displaying an image.

If the user terminal 300 has the form of a mobile terminal such as a smart phone, the user terminal 300 may further include a touch screen module capable of input and output as one module.

For example, the user terminal 300 may execute a medical examination-by-interview for the user to collect medical examination-by-interview result information about the medical examination from the user or output personal health information or current condition information of the user through the input unit 340 and the output unit 350.

The user terminal sensor 360 may detect information related to the health of the user, and may include, for example, an acceleration sensor and a location sensor. Accordingly, the user terminal 300 may obtain momentum information of the user through the user terminal sensor 360, for example.

FIG. 3 is a diagram for explaining an example of a configuration of the server 100 illustrated in FIG. 1 and an example of processing dosage form composition information.

The (a) of FIG. 3 is a diagram for explaining the configuration of the server 100, and (b) of FIG. 3 is a diagram for explaining a method of calculating dosage form composition information by receiving the current condition information by the server 100.

In FIG. 3 and subsequent drawings, detailed description of contents overlapping with those described in FIGS. 1 and 2 are omitted.

As illustrated in (a) of FIG. 3, the server 100 may include a communication unit 110, a memory 120, and a processor 130.

The communication unit 110 may perform communication with the device 200 and the user terminal 300 through a network under the control of the processor 130 to transmit information required for services provided by the server 100 to the device 200 and the user terminal 300 or receive the information from the device 200 and the user terminal 300.

The processor 130 may control the overall operation of the server 100. Specifically, the processor 130 may calculate the base dose information for each nutritional component or each cartridge from the personal health information of the user, and may calculate the dosage form composition information by reflecting the current condition information obtained from the user to the base volume information during or at the time point when the device 200 provides the dosage form to the user.

The memory 120 may store application information for driving the service provided by the server 100, information received from the user terminal 300 or device 200, and information generated during operation of the processor 130. Such a memory 120 may be provided in the form of various storage devices such as ROM, RAM, EPROM, flash drive, hard drive, etc. in terms of hardware, or may be provided in the form of a web storage performing a storage function of the memory 120 on the Internet or a storage form on the cloud.

As illustrated in (b) of FIG. 3, a driving method of the server 100 according to an example of the present invention may include the steps of receiving first current condition information (S111), calculating first dosage form composition information (S112), transmitting the first dosage form composition information (S113), receiving second current condition information (S114), calculating second dosage form composition information (S115), and transmitting the second dosage form composition information (S116).

In the step of receiving the first current condition information (S111), the processor 130 may control the communication unit 110 to receive the first current condition information about the user at a first time point, which is any one time point of the first period.

In the step of calculating the first dosage form composition information (S112), the processor 130 may calculate first dosage form composition information by reflecting the first current condition information.

More specifically, in the step of calculating the first dosage form composition information (S112), the processor 130 may calculate daily serving amount information in the first period by including amount information according to the first current condition information in the base dose information for each nutritional component or cartridge for the user, and may calculate the first dosage form composition information by converting the daily serving amount of the first period into the number of dosage forms to be dispensed for each cartridge from the device 200.

In the step of transmitting the first dosage form composition information (S113), the processor 130 may control the first dosage form composition information to be transmitted to the device 200 through the communication unit 110.

In the step of receiving the second current condition information (S114), the processor 130 may control the second current condition information about the user to be received by the communication unit 110 at a second time point, which is any one time point in the second period.

In the step of calculating second dosage form composition information (S115), the processor 130 may calculate second dosage form composition information by reflecting the second current condition information.

More specifically, in the step of calculating the second dosage form composition information (S115), the processor 130 may calculate daily serving amount information in the second period by including change amount information according to the second current condition information in the base dose information for each nutritional component or cartridge for the user, and may calculate the second dosage form composition information by converting the daily serving amount of the second period into the number of dosage forms to be dispensed for each cartridge in the device 200.

In the step of transmitting the second dosage form composition information (S116), the processor 130 may control the second dosage form composition information to be transmitted to the device 200 through the communication unit 110.

In each of the steps of receiving the first and second current condition information (S111 and S114), each of the first period and the second period may mean a length of a period predetermined in the server 100. As an example, each of the first and second periods may have a time length of 24 hours or a day length of 1 day.

The second period is a temporal concept located after the first period, and may mean a consecutive period or an inconsecutive period after the first period. For example, if the first and second periods are consecutive periods, and the first period means Jan. 1, 2022, the second period may mean Jan. 2, 2022. Or, as another example, if the first period is from 2:00 on Jan. 1, 2022 to 2:00 on January 2, the second period may be from 2:00 on Jan. 2, 2022 to 2:00 on January 3.

Each of the first and second time points may refer to a time point selected by a user in each of the first period and the second period. For example, the first time point may be 8:00 AM on January 1st, which is the first period, and the second time point may be 7:00 PM on January 2nd, which is the second period.

The first current condition information may refer to current condition information received by the server 100 during a first period or a first time point, and the second current condition information may refer to current condition information received by the server 100 during a second period or a second time point.

Here, as described above with reference to FIG. 1, each piece of current condition information may include information about at least one of (1) information about mental state or symptom of a user or (2) information about a physical state or symptom of a user. Specific contents are replaced with those described above in FIG. 1.

In addition, if there is no change in the body condition of the user, the user may select “none” as the current condition information, and in this case, the server 100 may receive “none” as the current condition information.

The server 100 may receive the first and second current condition information from the device 200 at the first and second time points, respectively. However, in some cases, it is also possible for the server 100 to receive the first and second current condition information from the user terminal 300 before the first and second time points during the first and second periods.

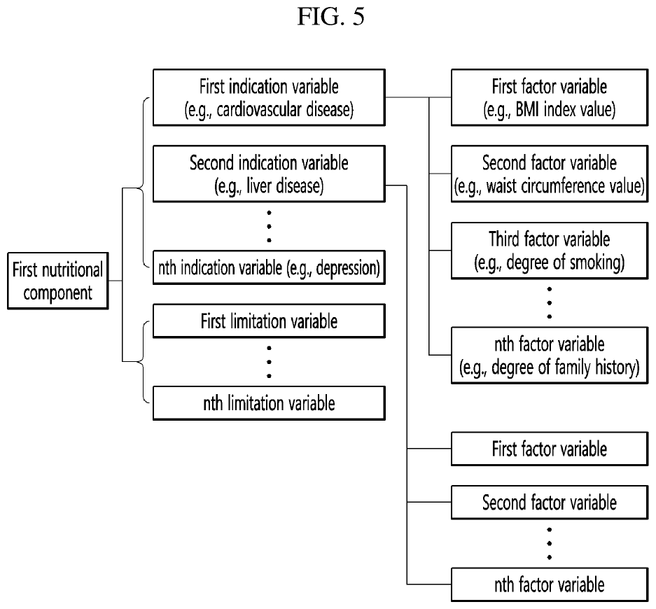

In each of the steps of calculating the first and second dosage form composition information (S112, S115), the base dose information may be dose information for each nutritional component or each cartridge calculated according to the personal health information of the user. The base dose information may be provided to the terminal as personalized base dose information for each nutritional component or each cartridge. A method of calculating the base dose information will be described later in detail with reference to FIGS. 4 and 5.

In each of the steps of calculating the first and second dosage form composition information (S112, S115), change amount information according to the current condition information may be dose information for each or nutritional component or each cartridge in which the current condition information is converted for each nutritional component. The change amount information may be included in the base dose information and calculated as daily serving amount information. A method of calculating the change amount information will be described later in detail with reference to FIG. 6.

In each of the steps of calculating the first and second dosage form composition information (S112, S115), the daily serving amount information is information obtained by including change amount information for each nutritional component or cartridge at the first or second time point in the base volume information, and may be dose information for each nutritional component provided to the user during the first period or the second period based on the personal health information and current condition information of the user.

Each of the first and second dosage form composition information may include information about the type and number of dosage forms to be dispensed from the device 200 corresponding to the first time point or the second time point.

Here, during the first and second periods, respective pieces of the base dose information used to calculate the first and second dosage form composition information are information based on the personal health information of the user, and may be identical to each other, and daily serving amount information for the first period according to the first current condition information obtained from the user at the first time point and daily serving amount information for the second period according to the second current condition information obtained from the user at the second time point may differ from each other.

Accordingly, when the second current condition information is different from the first current condition information, the second dosage form composition information may be different from the first dosage form composition information. That is, when the second current condition information is different from the first current condition information, at least one of the type and number of dosage forms according to the second dosage form composition information of the second time point may be different from at least one of the type and number of dosage forms according to the first dosage form composition information of the first time point.

In addition, in contrast, when the second current condition information is the same as the first current condition information, the content of the second dosage form composition information may be the same as the content of the first dosage form composition information, and the type and number of dosage forms according to the first dosage form composition information and the type and number of dosage forms according to the second dosage form composition information may be the same.

More specifically, there may be cases in which the server 100 receives ‘fatigue’ as the first current condition information at the first time point and receives ‘stress’ at the second time point as the second current condition information.

In this case, the numbers of a first dosage form containing a first nutritional component (vitamin B) related to fatigue dispensed at the first and second time points may be different from each other, and the numbers of a second dosage form containing a second nutritional component (e.g., lactic acid bacteria) related to stress dispensed at the first and second time points may be different from each other.

However, among the plurality of dosage forms, the numbers of a third dosage form containing a third nutritional component (e.g., Omega-3) not related to fatigue and stress dispensed at the first and second time points may be the same.

Hereinafter, a method of calculating the base dose information described above will be described in more detail with reference to FIGS. 4 and 5.

FIG. 4 is a diagram for explaining an example of a method for calculating the base dose information in the present invention. FIG. 5 is a diagram for explaining an example in which personal health variable value information is applied for each nutritional component when calculating the base dose information in the present invention.

In the present invention, the base dose information may be basically calculated by the server 100, but in some cases, the device 200 may also calculate the base dose information.

In FIG. 4 and subsequent drawings, a method of calculating base dose information by the server 100 is described as an example, but a method of calculating base dose information by the device 200 may also be applied similarly to FIG. 4.

As illustrated in FIG. 4, the method of calculating the base dose information by the server 100 may include a step of extracting initial variable value information (S121), a step of setting first base dose information (S122), a step of extracting personal health variable value information (S123), a step of calculating second base dose information (S124), a step of calculating third base dose information (S125), and a step of calculating fourth base dose information (S126).

The base dose information calculated as illustrated in FIG. 4 may be calculated differently for each nutritional component according to the personal health information of each user, and the result calculated as the base dose information may be provided to the user as personalized base dose information for each nutritional component or each cartridge. A program for calculating the base dose information may be provided in the form of a program module or an artificial intelligence (AI) program module in the memory of the server 100 or the device 200 terminal.

In addition, in FIG. 4, a case in which each step is sequentially performed is illustrated as an example, but the present invention is not limited thereto. The step of extracting initial variable value information (S121) and the step of extracting personal health variable value information (S123) may be performed in advance before calculating the base dose information.

In addition, the method of calculating the base dose information according to the present invention is not necessarily limited to FIG. 4, and in some cases, at least one of the steps of calculating the second, third, and fourth base dose information among the steps illustrated in FIG. 4 may be omitted. However, in the case of including the steps of calculating the second, third, and fourth base dose information, the base capacity can be calculated more precisely for the personal health of the user. Accordingly, hereinafter, the case in which the steps of calculating second, third, and fourth base dose information are included will be described as an example.

In the step of extracting initial variable value information (S121), at least one initial variable value information may be extracted from the personal health information received by the server 100.

Here, the personal health information may be extracted from at least one of user identification information, medical examination-by-interview result information, or medical information. Hereinafter, as an example, a case in which identification information about a user, medical examination-by-interview result information, and medical information are all extracted will be described as an example.

The identification information about the user may include, for example, information about a subscription ID, name, contact information, gender, and age of the user. Such user identification information may be collected when the user signs up for a membership of a service provided by the server 100.

The medical examination-by-interview result information may refer to information about a result of the user terminal 300 or the device 200 performing a medical examination-by-interview to the user. To this end, the server 100 may transmit medical examination-by-interview inquiry to the user terminal 300 or the device 200 so that the user terminal 300 or the device 200 may perform the medical examination-by-interview.

The medical examination-by-interview inquiry information may include basic inquiry information and detailed inquiry information. The basic query information may include, for example, basic items such as the gender, age, height, weight, pregnancy status, breastfeeding status, etc. of the user, and the detailed inquiry information may include, for example, detailed items classified by category.

For example, the detailed inquiry information may include a plurality of categories including first, second, and third categories. For example, each of the first, second, and third categories may include detailed items.

For example, the detailed items of the first category may include questions about drinking, smoking, exercise, sleeping habits, eating habits, etc., the detailed items of the second category may include questions about self-efficacy, work-related, immunity-related (allergies, etc.), drugs being taken (e.g. pharmaceuticals or nutrients), etc., or the detailed items of the third category may include questions about indicants (or indications) indicating various kinds of physical or mental diseases, symptoms or conditions. For example, the detailed items of the third category may include questions related to physical indications such as cardiovascular disease, liver disease, cancer-related disease, nervous system disease, family history of illness or disease, immunity, and stress and and questions related to mental indications such as depression, insomnia, and anxiety.

In addition, the detailed inquiry information of the medical examination-by-interview information may include additional inquiry information when there is a selection response corresponding to a specific category, and the additional inquiry information may include additional query information about the selected indication. For example, if the user responds to an indication of cardiovascular disease, in relation to the factors that form cardiovascular disease, additional inquiry information such as duration of illness, treatment method, current or past history, complications, and severity of symptoms may be further included.

As another example, the medical examination-by-interview inquiry information may include (1) physical information (e.g., age, height, weight, body mass index (BMI), etc.) of a user, (2) drugs that the user is taking, (3) health functional food that the user is taking, (4) lifestyle of the user, (5) a disease that the user has or has a history of having, and (6) question information about a symptom that the user has or has a history of having.

Using the medical examination-by-interview inquiry information as described above, the user terminal 300 or the device 200 may perform a medical examination-by-interview for the user.

When performing a medical examination-by-interview, the user terminal 300 or device 200 may display, for example, medical examination-by-interview inquiry information through a touch screen module capable of input and output with one module, store the value input through the interaction of the user as the medical examination-by-interview result information and transmit the value to the server 100. Alternatively, as another example, the user terminal 300 or device 200 may output medical examination-by-interview inquiry information by voice and receive the user's voice as medical examination-by-interview result information.

The medical information is information received by the server 100 from the medical institution server 500, and may include, for example, at least one of medical record information, image capture information, drug-taking information, or health checkup result information that the user has been tested, examined, or diagnosed at a medical institution.

The server 100 may extract initial variable value information from personal health information extracted from at least one of information, medical the identification examination-by-interview result information, or medical information about the user as described above.

Here, the initial variable value information includes the gender and age of the user, and may optionally further include the pregnancy status and breastfeeding status of the user.

In the step of setting the first base dose information (S122), the server 100 may set first base dose information as dose information for each nutritional component or each cartridge based on the initial variable value information extracted from the personal health information.

Specifically, the server 100 may set the minimum dose for each nutritional component or cartridge for the user by the inputting initial variable value information for the user as an input value into a base dose table (or base dose algorithm). This minimum dose may be an initial setting value for calculating the base dose information.

For example, the server 100 may calculate dose information for each nutritional component or cartridge by inputting variable value information about the gender, age, pregnancy status, and breastfeeding status of the user into the base dose table.

In the step of extracting personal health variable value information (S123), the server 100 may extract personal health variable value information including at least one of indication variable value information, factor variable value information, and limitation variable value information from the personal health information. For example, the personal health variable value information includes the indication variable value information and the factor variable value information, and may optionally include the limitation variable value information.

The indication variable may be a variable for an indication indicating a physical illness, disease, symptom or condition, a mental illness, disease, symptom or condition, or energy state affected by nutritional component or cartridge component. The indication variable value information may be information about values of indication variables extracted from the user's personal health information.

As illustrated in FIG. 5, such an indication variable may include a plurality of indication variables (e.g., a first indication variable to an nth indication variable) for each nutritional component or each cartridge. Examples of such indication variables may include cardiovascular disease, liver disease, depression, etc.

The factor variable is a variable that affects each indication variable, and may be a variable for a detailed factor that forms or changes the value of the indication variable. The factor variable value information may be information about values of factor variables for a specific indication variable.

The factor variable may be set for each indication variable. Therefore, a plurality of factor variables may be dependent on one indication variable, and a plurality of pieces of factor variable value information may be summed with a weight to form an indication variable value.

As illustrated in FIG. 5, when the first indication variable is, for example, cardiovascular disease, the factor variable is a variable that affects or forms the degree of cardiovascular disease. For example, the BMI index value, waist circumference value, smoking level, family history level, etc. of the user may be used as the factor variables.

Such an indication variable and a factor variable may be used to increase or decrease the first base dose information calculated for each nutritional component or each cartridge. For example, a plurality of indication variables may be included with a weight for the first nutritional component illustrated in FIG. 5 to increase the first base dose of the first nutritional component and may be used to calculate the second base dose information.

As illustrated in FIG. 5, the limitation variable may be a variable for a factor that is prohibited or restricted from taking for each nutritional component or cartridge according to the health condition of the user, corresponding to the indication variable. The limitation variable value information may be information about values of limitation variables extracted from personal health information of the user.

Such limitation variables may include (1) lifestyle (e.g., smoking, sleeping hours, etc.) or dietary habits (e.g., mackerel consumption, etc.) of the user, (2) condition of the user (e.g., pregnancy, breastfeeding, etc.), (3) drug the user is taking, or (4) illness or disease of the user, etc. It can be used to calculate the second base dose information by decreasing the first base dose of the nutritional component by the limitation variable.

For example, with regard to the limitation variable, in the present invention, when a user takes a specific drug, the server 100 may limit or not provide a serving amount of a specific dosage form in consideration of the interaction between the specific dosage form provided by the device 200 and the specific drug.

In addition, when the user has a specific disease or illness, the server 100 may limit or not provide a serving amount of a specific dosage form in consideration of the interaction between the specific dosage form provided by the device 200 and the specific specific disease or illness.

In the present invention, a case in which all of the indication variable value information, factor variable value information, and limitation variable value information are applied is described as an example, but, differently from this, it is also possible that only the indication variable value information and the factor variable value information are applied and the limitation variable value information is not applied. However, when all of the information are applied, it is possible to provide a more precisely customized dosage form to the personal health information of the user, and the case where all of the information are applied will be described as an example.

Such indication variable value information, factor variable value information, and limitation variable value information may be expressed as numerical values extracted from personal health information of the user.

For example, in the personal health information of the user including at least one of user identification information, medical information, and medical examination-by-interview result information about the user, when it is recognized that cardiovascular disease exists, an indication variable for cardiovascular disease may be selected, and factor variable value information may be extracted according to a numerical value of body mass index (BMI), a numerical value of waist circumference, a numerical value of smoking amount, a numerical value of family history, etc., which affect cardiovascular disease.

In the step of calculating the second base dose information (S124), the server 100 may calculate the second base dose information by reflecting the personal health variable value information extracted from the personal health information in the first base dose information.

In the step of calculating the second base dose information (S124) of the present invention, the second base dose information for each nutritional component or each cartridge may be calculated in consideration of at least one of (1) the interaction between the physical information of the user (e.g., age, height, weight, body mass index (BMI), body surface area, pregnancy, genetic information, etc.) and the nutritional component to be provided, (2) the interaction between the drug being administered and the nutritional components to be provided, (3) the interaction between the nutrients being taken and the nutritional components to be provided, (4) the interaction between the disease the user has and the nutritional components to be provided, (5) the interaction between the lifestyle and nutritional components to be provided, and (6) the interaction between the symptom the user has and the nutritional components to be provided.

To this end, the server 100 may calculate the second base dose information by varying the first base dose information for each nutritional component or each cartridge according to personal health variable value information.

More specifically, the server 100 may reflect the factor variable value information dependent on each indication variable as an input value in the variable value-nutritional component mapping table stored in advance in the memory 120, calculate it according to a predetermined formula, and calculate a detailed change rate value according to each factor variable value information. Here, the factor variable value information may be one of personal health variable value information extracted from the personal health information.

Thereafter, a change rate value for each indication variable may be calculated by applying a multiplication operation, a sum operation, etc. to the detailed change rate of each factor variable through a program module or an artificial intelligence (AI) program module.

Here, the change rate value of each indication variable may have a value increasing the first base capacity.

For reference, the change rate value of each indication variable calculated based on the variable value-nutritional component mapping table may be calculated within a range of the maximum change rate R value set for each indication variable. In addition, a detailed change rate r according to each factor variable value may be calculated within a range of the change rate smaller than a maximum change rate R of each indication variable.

In addition, the limitation variable value information may calculate the second base dose by decreasing the first base dose, which is increased by indication change rate value, by a predetermined dose or processing it back to 0 (zero).

Accordingly, the indication variable may be used to calculate the dose of each nutritional component that has a positive effect on a physical disease or symptom, a mental disease or symptom, or energy condition, the factor variable may be used to control the increase of the indication variable, and the limitation variable may be used as a variable that decreases the dose of each nutritional component or make it 0 (zero).

For example, in the case of the limitation variable, when it is confirmed that sufficient intake of the first nutritional component is obtained through food or outdoor activities, the value may be set to the sum of negative numbers, and the dose determined by the indication variable and the factor variable may be decreased according to the application of the limitation variable.

Further, in a case where the nutritional component is already being taken as a prescription drug or should not be taken due to an interaction with a specific drug being taken according to a prescription, or in a case where the nutritional component is a nutritional component that is contraindicated according to age, a nutritional component that is contraindicated for pregnant or breast-feeding women, or a nutritional component that is contraindicated for taking due to other health conditions such as a preexisting disease, the value of the limitation variable may be set by a multiplication operation of 0 (zero), and accordingly, the dose determined by the indication variable and the factor variable may be converted to 0 (zero) and limited.

More specifically, for example, the indication variables for Omega-3 may include (1) cardiovascular disease, (2) pregnancy-related disease or syndrome, (3) breast-feeding-related disease or syndrome, (4) liver disease, etc. as physical diseases or symptoms, etc. positively affected by Omega-3, and include (1) cognitive dysfunction, (2) depression, (3) bipolar disorder, etc. as mental diseases or symptoms positively affected by Omega-3.

In addition, when the indicator variable for Omega-3 is cardiovascular disease, the factor variables for cardiovascular disease may include body mass index (BMI), waist circumference, smoking, drinking, lack of exercise, and interaction with drugs (drugs that induce Hyper TG), triglyceride level, family history, menopause information, which are CVD risk factors, etc. as factors affecting cardiovascular disease.

In addition, the limitation variable for Omega-3 is a negative variable, which may include, for example, the degree of intake of mackerel as a variable, and as a variable that prohibits the intake of Omega-3 or limits it 0 (zero), taking prohibited prescription drugs (e.g., drug-nutrient interaction (DNI)), whether or not to take Omega-3 separately, etc. may be set.

The first base dose according to the personal health information of the user, the amount of increase by the indication variable and the factor variable, and the amount of decrease or limitation by the limitation variable described above may be recorded and updated in a variable value-nutritional component mapping table.

The server 100 of the present invention may calculate the second base dose information from the first base dose information by calculating the indication variable, the factor variable, and the limitation variable with reference to the variable value-nutritional component mapping table.

Here, the variable value-nutritional component mapping table may be configured by assigning a maximum change rate R corresponding to each sequential variable process.

For example, in the variable value-nutritional component mapping table, the maximum change rate range R for each indication variable is set corresponding to the first base dose for each nutritional component or each cartridge, and the factor variable may be assigned an increase weight applied within the maximum change rate range R.

In addition, when a plurality of indication variables are selected, the second base dose for each nutritional component may be individually calculated for each indication variable. The second base dose for each nutritional component or each cartridge may be determined by applying and summing weights (S1, S2, S3, . . . , SN) in ascending order of change rate values (R1, R2, R3, . . . RN) for each indication variable calculated by the variable value-nutritional component mapping table.

More specifically, when the change rates for a plurality of indication variables are calculated by the variable value-nutritional component mapping table, as shown in Equations 1 and 2 below, the second base dose for the corresponding nutritional component may be calculated by applying weights (S1, S2, S3, . . . , SN) to change rates of the plurality of indication variables and multiplying each weight by the first base dose.

For example, as shown in Equations 1 to 4 below, an increase value of the first base dose information may be calculated using the indication variable value information and the factor variable value information. However, this is an example and the present invention is not necessarily limited to Equations 1 to 4.

D(RAW)=D₀×S [Equation 1]

Here, D0 means the first base dose for each nutritional component, S means the sum of the weights for the corresponding nutritional component, and D (raw) may mean the second base dose for each nutritional component to be calculated.

S=S₁+S₂+S₃+ . . . +Sₙ [Equation 2]

Here, S1, S2, S3, . . . , Sn each mean a weight value for each indication variable, and the total sum of the weights (S) may be calculated by summing the weight values for each indication variable.

Here, the respective weight values (S1, S2, S3, . . . , Sn) for each indication variable may be values in which change rate values (R1, R2, R3, . . . RN) for each indication variable are arranged in ascending order.

Here, the weight values (S1, S2, S3, . . . , Sn) for each indication variable may be calculated as in Equations 3 and 4 below.

S₁=R₁ [Equation 3]

In Equation 3, S1 means the largest weight value among weight values of a plurality of indication variables, and R1 may mean the largest change rate value R1 among the change rate values (R1, R2, R3, . . . RN) according to a plurality of indication variables calculated by the variable value-nutritional component mapping table.

Therefore, in the present invention, among the change rate values of a plurality of indication variables, the largest change rate value R1 may be assigned as the largest weight value S1.

$$[ Equation 4 ]$$

$${S}_{n} ( weight value of each indication ) = ( {R}_{n} - 1 ) \times \frac{1}{{\sum }_{r = 1}^{n - 1} {S}_{r}} ( n \geq 2 )$$

Equation 4 may be an equation for determining weight values of next rank (S2, S3, . . . Sn) excluding the largest weight value S1 among the weight values of the plurality of indication variables.

In Equation 4, the value of n has a value greater than 2, Rn is the change rate value of each indication variable, and the calculated change rate value of Rn may be determined in descending order from the largest value. Sr means a weight value for each indication variable.

Therefore, for example, assuming that the calculated change rate value for the first indication variable has a value of 1.15 at 115%, the calculated change rate value for the second indication variable has a value of 2.2 at 220%, the calculated change rate value for the third indication variable has a value of 1.2 at 120%, and the calculated change rate value for the fourth indication variable has a value of 1.5 at 150%, R1 may be 2.2, which is a value of the change rate value of the second indication variable having the largest value, R2 may be 1.5, which is the next highest change rate value, R3 may be 1.2, and R4 may be 1.15.

Accordingly, R1=2.2, R2=1.5, R3=1.2, R4=1.15 are reflected in Equations 3 and 4 to calculate the weights S1, S2, S3, and S4 of each indication variable, and the total sum weight value S may be calculated according to Equation 2.

Thereafter, according to Equation 1, the S value, which is the total sum weight value, is reflected in the first base dose for each nutritional component, and the decrease amount according to the limitation variable value information is reflected for each nutritional component, so that the second base dose for each nutritional component may be calculated.

In the step of calculating the third base dose information (S125), the server 100 may calculate the third base dose information by cutting off the dose of nutritional components exceeding the first maximum serving amount of the second base dose information to the first maximum serving amount.

Further, in the step of calculating the third base dose information (S125), along with the correction of the first maximum serving amount described above, nutritional components that do not meet the preset minimum serving amount among the second base dose information may be corrected to have a minimum serving amount. However, when performing the correction of the minimum serving amount, correction for nutritional components prohibited from taking in personal health information may be excluded.

Here, the first maximum serving amount may mean the maximum recommended amount for each nutritional component or each cartridge that the user can take for a predetermined period of time. That is, if a user takes too much of a nutritional component, a desired effect may not occur or side effects may occur. In order to prevent this, the first maximum serving amount may be set for each nutritional component or each cartridge.

The predetermined period may be 1 day or 24 hours, the first maximum serving amount may be the maximum recommended daily amount for each nutritional component or each cartridge. However, the present invention is not necessarily limited thereto, and it is possible that the predetermined period may vary for each nutritional component or each cartridge.

Therefore, in the step of calculating the third base dose information (S125), when the second base dose, in which the personal health variable value information is reflected for each nutritional component or each cartridge, exceeds the maximum recommended daily amount, the server 100 may calculate the third base dose by performing a cut-off correction of the second base dose to a range of the maximum recommended daily amount for the dose for each nutritional component or the dose for each cartridge that exceeds the maximum recommended daily amount.

The first maximum serving amount for each nutritional component or each cartridge may be set differently according to demographic group information (such as gender, age, pregnancy status and breast-feeding status), health condition information, lifestyle information, side effects for each nutritional component, etc.

More specifically, the first maximum serving amount for each nutritional component or each cartridge may be set differently according to demographic group information (such as gender, age, pregnancy status and breast-feeding status).

For example, for vitamin C, the first maximum serving amount may be set to 200 mg for men between the ages of 40 and 50, and the first maximum serving amount may be set to 150 mg for women between the ages of 15 and 19.

Therefore, when the second base dose for vitamin C for a first user was calculated as 170 mg, (1) if the first user is a male between the ages of 40 and 50, 170 mg is calculated as the third base dose, but (2) if the first user is a female between the ages of 15 and 19, the third base dose may be calculated by performing a cut-off correction of the vitamin C dose to 150 mg.

In addition, the first maximum serving amount for each nutritional component or each cartridge may be set differently according to the personal health information of the user (physical or mental disease or condition), set differently according to lifestyle of the user (smoking status, etc.), or set differently according to side effects according to the corresponding nutritional component.

For example, if the second base dose for vitamin C is calculated as 170 mg for the first user, but the first user has gastrointestinal disorder, the dose of vitamin C may be cut off to 100 mg by the first maximum serving amount according to gastrointestinal disorder and calculated as a third base dose. In addition, unlike this, if the first user has urinary stones, the dose of vitamin C may be cut off to 50 mg by the first maximum serving amount according to urinary stones and calculated as a third base dose.

As described above, if the first maximum serving amount corresponds to a plurality of cases (e.g., the first user is a male between the ages of 40 and 50 with gastrointestinal problems and urinary stones), the first maximum serving amount (e.g., gastrointestinal disorders-200 mg, urinary stones-50 mg) may be selected as the smallest value (e.g., 50 mg).

In the step of calculating the fourth base dose information (S126), the server 100 may allocate any one base dose information of the first to third base dose information to each cartridge based on the cartridge information for each of the plurality of cartridges received from the device 200, and perform redundant correction to calculate the fourth base dose information for each cartridge. Here, any one base dose information of the first to third base dose information may mean base dose information calculated before the step of calculating the fourth base dose information (S126).

That is, when the server 100 has calculated up to the first base dose information but has not calculated the second and third base dose information, the first base dose information may be allocated for each cartridge, when the server 100 calculates up to the second base dose information, the second base dose information may be allocated for each cartridge, and when the server 100 calculates up to the third base dose information, the third base dose information may be allocated for each cartridge.

The cartridge information may be received by the server 100 from the device 200 and stored in the memory 120 of the server 100 before performing the step of calculating the fourth base dose information (S126). If the server does not obtain cartridge information at the time point of calculating the fourth base dose information (S126), the corresponding step (S126) may be omitted or the corresponding step (S126) may be performed for all types of cartridges stored in the server. If the corresponding step (S126) is omitted, the server 100 may provide base dose information calculated before the corresponding step (S126) to the user terminal 300 as personalized basic capacity information for each nutritional component or each cartridge.

When the server 100 performs step of calculating the fourth base dose information (S126), the cartridge information may include information about each of a plurality of cartridges currently loaded in the device 200 and information about dosage forms stored for each cartridge.

For example, when the number of cartridges currently loaded in the device 200 is eight, the cartridge information may include information about dosage forms provided in each of the eight cartridges. The information about the dosage form may include information about the type of dosage form provided in the cartridge, the number of remaining amounts of the dosage form, the date of manufacture of the dosage form, and information about the manufacturer.

Information about the type of dosage form may include the name or identification number of the dosage form. When the server 100 receives information about the type of dosage form, the server 100 may query a DB of the dosage forms stored in the memory to check information about a unit weight of the corresponding dosage form, nutritional components contained in the dosage form, and content of nutritional components per unit weight of the dosage form.

Therefore, the server 100 may query cartridge information to recognize or check what dosage form of nutritional components is currently stored in the device 200 for each cartridge, the content or content ratio of nutritional components contained per each dosage form, and the total number of dosage forms and the total amount of nutritional components.

Accordingly, the server 100 may query the cartridge information to recognize nutritional components contained per unit dosage form and the content or content ratio of the corresponding nutritional component for the dosage form stored in each cartridge.

The redundant correction may mean that, when the server 100 queries cartridge information and recognizes that the same nutritional component exists in a plurality of cartridges, a dose of the nutritional component allocated to at least one cartridge among a plurality of cartridges containing the same nutritional component is subtracted.

For example, there may be a case where the third base dose calculated for iron powder is 50 mg and the third base dose calculated for the magnesium component is 10 mg, and as a result of cartridge information query of the server 100, an iron cartridge containing iron powder (100%) and iron powder (50%) and magnesium (50%) exist together.

In this case, the server 100 may first allocate 10 mg of the third base dose for the magnesium component to the multi-mineral cartridge.

Thereafter, the server 100 may calculate 40 mg as the fourth base dose of the iron component in the iron cartridge by performing redundant correction that subtracts 10 mg of the iron component quota, which occurs when allocating 10 mg of magnesium component to the multi-mineral cartridge, from the third base dose of 50 mg calculated for the iron component.

For reference, the fourth base dose information for the multi-mineral cartridge may be calculated as 20 mg, which is a sum of 10 mg of magnesium and 10 mg of iron.

As another example, there may be a case where the third base dose calculated for iron is 50 mg and the third base dose calculated for multi-mineral containing both iron (50%) and magnesium (50%) is 80 mg, and, as the result of cartridge information query of the server 100, the iron cartridge and the multi-mineral cartridge exist together.

In this case, the server 100 may first allocate a third base dose of 80 mg to the multi-mineral cartridge.

Thereafter, the server 100 may calculate 10 mg as the fourth base dose of the iron component in the iron cartridge by performing redundant correction that subtracts 40 mg of the iron component quota, which occurs when allocating 80 mg to the multi-mineral cartridge, from the third base dose of 50 mg calculated for the iron component.

As another example, there may be a case where the third base dose calculated for iron is 40 mg and the third base dose calculated for multi-mineral containing both iron (50%) and magnesium (50%) is 80 mg, and, as the result of cartridge information query of the server 100, the iron cartridge and the multi-mineral cartridge exist together.

In this case, the server 100 may first allocate a third base dose of 80 mg to the multi-mineral cartridge.

Thereafter, the server 100 may calculate 0 mg as the fourth base dose of the iron component in the iron cartridge by performing redundant correction that subtracts 40 mg of the iron component quota, which occurs when allocating 80 mg to the multi-mineral cartridge, from the third base dose of 40 mg calculated for the iron component. In other words, in this case, the quota for the iron component may be satisfied only with the multi-mineral component. This may be the case even when the third base dose for iron is less than 40 mg. Since it is not possible to provide a nutritional component with a negative (−) value, the fourth base dose will be calculated as 0 mg, not a negative (−) value.

Such redundant correction may be applied not only between a cartridge containing a single nutritional component (e.g., contains iron) and a cartridge containing a complex nutritional component (e.g., contains iron and magnesium), but also between a first complex nutritional component cartridge (e.g., containing zinc and vitamin C) and a second complex nutritional component cartridge (e.g., containing zinc, vitamin C and vitamin E).

The server 100 may calculate the fourth base dose information by performing the redundant correction described above.

Here, the first, second, third, and fourth base dose information described above may have a unit value according to each nutritional component, such as international unit (OS), milligram (mg), microgram (mcg), or colony forming unit (CFU.)

However, the present invention is not necessarily limited thereto. Since the fourth base dose information calculated by the server 100 is information assigned to nutritional components of dosage forms in which the third base dose information for each nutritional component or cartridge is held by each cartridge, it is also possible that the fourth base dose information for each nutritional component or cartridge has a unit value of weight (e.g., mg).

As described above, the server 100 of the present invention may calculate the base dose differently according to the personal health information of the user. That is, according to the present invention, the base dose of each nutritional component or each cartridge may vary according to the health condition of the user for each user. Therefore, the base dose information calculated according to the present invention may be personalized base dose information for each nutritional component or each cartridge optimized according to the health condition of the user.

So far, the case where the server 100 calculates the base dose information has been described as an example, but the present invention is not limited thereto, and the base dose information may be calculated by the device 200 in some cases.

To this end, a program or algorithm for calculating base dose information may be stored in the device 200 as well.

If the device 200 calculates the base dose information, the steps of calculating the first, second, third, and fourth base dose information in FIG. 4 are maintained as they are, and initial variable value information and personal health variable value information may be received from the server 100 instead of extracting the initial variable value information and the personal health variable value information.

FIG. 6 is a diagram for explaining an example in which current condition information is reflected in the base dose information in the present invention.

In (a) of FIG. 6, a method reflecting the current condition information of the user to the base dose information by the server 100 will be described as an example, but the present invention is not necessarily limited thereto, and it is also possible for the device 200 to reflect the current condition information of the user to the base dose information.

In order to perform an operation of reflecting the current condition information to the base dose information, a program or artificial intelligence (AI) program for reflecting the current condition information may be provided in the memory of the server 100 or the device 200 terminal.

The server 100 of the present invention may provide a dosage form having a dosage more optimized for the real-time body condition change of the user by reflecting the current condition information of the user in the base dose information calculated based on the personal health information of the user.

Here, the time point at which the current condition information of the user is reflected in the base dose information may be at or before the time point at which the user receives the dosage form.

To this end, the server 100 may receive current condition information of the user from the device 200 or the user terminal 300. The time point at which the server 100 receives the current condition information of the user may be a time point at which the user receives a dosage form or a period including the time point at which the user receives the dosage form.

Specifically, the time point at which the user receives the dosage form may be the first and second time points described above, or a period from the start of the first period to the first time point, or a period from the start of the second period to the second time point.

As illustrated in (a) of FIG. 6, a method in which the server 100 reflects the current condition information of the user in the base dose information may include the steps of inputting (S131), calculating change amount information (S132), calculating daily serving amount (S133), and converting to the number of dosage forms (S134).

In the step of inputting (S131), the current condition information received by the server 100 may be input to a condition reflection algorithm.

The condition reflection algorithm may receive current condition information and calculate a value capable of being varied for each nutritional component or cartridge according to the current condition information. The condition reflection algorithm may be stored in the memory 120 of the server 100, and may be called and used at a necessary time point under the control of the processor 130.

In the step of calculating the change amount information (S132), the condition reflection algorithm provided in the server 100 may calculate the change amount information for each nutritional component or each cartridge based on the current condition information of the user.

For example, the condition reflection algorithm may reflect the current condition information of the user based on the third base dose information described above to calculate change amount information for each nutritional component or each cartridge.

In the present invention, the case where the change amount information according to the current condition information is calculated based on the third base dose information is described as an example, but the present invention is not necessarily limited thereto, and it is also possible to calculate the change information amount based on the first base dose information or the second base dose information. However, when the server 100 calculates the change amount information based on the third base dose information for which the first maximum serving amount correction has been performed, the current condition information of the user may be more precisely reflected. Hereinafter, a case in which the change amount information is calculated based on the third base dose information will be described as an example.

Therefore, in FIG. 3 described above, when the server 100 calculates the change amount information according to the first current condition information at the first time point or the change amount information according to the second current condition information at the second time point, the change amount information according to each of the first and second current condition information may be calculated differently for each nutritional component or for each cartridge according to the third base dose information.

Specifically, it is assumed that the device 200 has a plurality of cartridges storing first, second, and third dosage forms that respectively contain first, second, and third nutritional components, the first nutritional component affects first current condition information, the second nutritional component affects second current condition information, and the third nutritional component does not affect the first and second current condition information.

In such a case, when the server 100 receives the first current condition information at the first time point, the server 100 may calculate change amount information having a positive (+) value or a negative (−) value only for the first nutritional component, and calculate change amount information in which each change amount is 0 (zero) for the second and third nutritional components.

In addition, when the server 100 receives the second current condition information at the second time point, the server 100 may calculate information having a positive (+) value or a negative (−) value only for the second nutritional component, and calculate information in which the value of change amount is 0 (zero) for the first and third nutritional components.

For example, it is assumed that the cartridge of the device 200 contains a lactic acid bacteria component, a multi-mineral component, and an Omega-3 component, the lactic acid bacteria component affects only ‘stress’, the multi-mineral component affects only ‘exercise’, and the Omega-3 component has no effect on stress and exercise

In such a state, when the server 100 receives information about ‘stress’ as the first current condition information, the server 100 may calculate change amount information having a positive (+) value or a negative (−) value only for the lactic acid bacteria component related to stress, and calculate information in which the change amount is 0 (zero) for the Omega-3 component and the multi-mineral component.

In addition, when the server 100 receives information about ‘exercise’ as the second current condition information, the server 100 may calculate change amount information having a positive (+) value or a negative (−) value only for the multi-mineral component related to exercise, and calculate information in which the change amount is 0 (zero) for the Omega-3 component and the lactic acid bacteria component.

In addition, the change amount having a positive (+) value in the change amount information may be calculated differently according to a size value of each nutritional component or the third base dose of the cartridge. For example, the change amount having a positive (+) value may be calculated differently according to the ratio of the third base dose to the second maximum serving amount for each nutritional component or each cartridge.

Here, the second maximum serving amount may be information stored in the memory 120 in advance of the server 100, and can have a value equal to or greater than the first maximum serving amount described above. In the present invention, a case where the second maximum serving amount for each nutritional component or each cartridge is the same as the first maximum serving amount will be described as an example. For example, the second maximum serving amount, like the first maximum serving amount, may be set differently according to the demographic group (gender, age, pregnancy status, breast-feeding status) for each nutritional component or each cartridge and physical or mental disease, symptom, or condition for each nutritional component or each cartridge.

More specifically, (1) when the ratio of the third base dose to the second maximum serving amount is large, an allowable width of an increased change amount of may be relatively small, and when the ratio of the third base dose to the second maximum serving amount is small, the allowable width of the increased amount may be relatively large.

For example, if the second maximum serving amount of the user for the vitamin C component is 200 mg and the third base dose of the user for the vitamin C component is 150 mg, the allowable width of the change amount of the vitamin C according to the current condition component increased information may be increased within a range of 50 mg.

However, unlike the matters described above, if the second maximum serving amount of the user for the vitamin C component is 200 mg or the third base dose of the user for the vitamin C component is 50 mg, the allowable width of the change amount of the vitamin C component increased according to the current condition information may be increased within a range of 150 mg.

As described above, after the change amount information according to the current condition information is calculated, when the amount obtained by adding the change amount to the third base dose for each nutritional component or each cartridge exceeds the second maximum serving amount, the server may perform correction to cut off the calculated change amount to a limit of the second maximum serving amount so that the amount obtained by the addition does not exceed the second maximum serving amount.

That is, the server 100 may adjust the change amount calculated through correction of the second maximum serving amount, and adjusted change amount information may be used in the step of calculating the daily serving amount.

In the step of calculating the daily serving amount (S133), the server 100 may calculate the daily serving amount by including the change amount information in the base dose information for each nutritional component or each cartridge.

Here, the base dose information used to calculate the daily serving amount may be fourth base dose information calculated for each cartridge by applying redundant correction thereto.

Here, since the daily serving amount is calculated by reflecting the change amount information, in which the current condition information of the user is reflected, in the base dose information, the daily serving amount may be calculated differently for each time point at which the current condition information is received.

More specifically, when the first current condition information described in FIG. 3 and the second current condition information are different from each other, daily serving amount information for each nutritional component or each cartridge according to the first current condition information at the first time point during the first period may be different from daily serving amount information for each nutritional component or each cartridge according to the second current condition information at the second time point during the second period.

However, unlike the matters described above, when the first current condition information and the second current condition information are equal to each other, the daily serving amount information for each nutritional component or each cartridge according to the first current condition information and the daily serving amount information for each nutritional component or each cartridge according to the second current condition information may be equal to each other.

For example, it is assumed that, as shown in (b) of FIG. 6, the cartridge of the device 200 contains a lactic acid bacteria component, a multi-mineral component, and an Omega-3 component, the lactic acid bacteria component affects only ‘stress’, the multi-mineral component affects only ‘exercise’, the Omega-3 component has no effect on stress and exercise, and 7 billion CFU of lactic acid bacteria component component, 100 mg of multi-mineral component, and 120 mg of Omega-3 component are calculated as the fourth base dose according to the personal health information of the user.

Here, when the server 100 receives stress and exercise as the first current condition information, and calculates the change amount of the lactic acid bacteria component as an increase of 3 billion CFU, the change amount of the multi-mineral component as an increase of 50 mg, and the change amount of Omega-3 as 0 (zero), the server 100 may calculate 10 billion (7 billion+3 billion) CFU of lactic acid bacteria component, 150 mg (100 mg+50 mg) of multi-mineral component, and 120 mg (120 mg+0 mg) of Omega-3 component as daily serving amount for each nutritional component or each cartridge according to the first current condition information.

In addition, when the server 100 receives stress as the second current condition information, and calculates the change amount of the lactic acid bacteria component as an increase of 3 billion CFU and the change amount of the multi-mineral component and Omega-3 as 0 (zero), the server 100 may calculate 10 billion (7 billion+3 billion) CFU of lactic acid bacteria component, 100 mg (100 mg+0 mg) of multi-mineral component, and 120 mg (120 mg+0 mg) of Omega-3 component as daily serving amount for each nutritional component or each cartridge according to the second current condition information.

In addition, as an example, when the current condition information is not obtained from the user terminal 300 or the device 200 or is selected as ‘none’ and then the server 100 receives it, the server 100 may calculate the value of the change amount according to the current condition information as 0 (zero).

If the change amount value is 0 (zero), since the change amount information of each nutritional component or each cartridge included in the base dose information for each nutritional component or each cartridge is 0 (zero), the server 100 may calculate the same value as the fourth base dose information for all nutritional components or cartridges as daily serving amount information. For example, when the server 100 receives ‘none’ as the current condition information, the server 100 can calculate 7 billion CFU of lactic acid bacteria component, 100 mg of multi-mineral component, and 120 mg of Omega-3 component in the same amount as the fourth base dose, as the daily serving amount.

In addition, if the change amount of any nutritional component or cartridge has a negative (−) value, the server 100 may calculate the daily serving amount by subtracting the change amount calculated from the fourth base dose information of the corresponding nutritional component or cartridge or by decreasing the fourth base dose information by a ratio according to the change amount.

For example, if the fourth base dose of the vitamin C component causing gastrointestinal disorder is 100 mg, and gastrointestinal disorder is received as the current condition information and the change amount of vitamin C component is calculated as (−) 50 mg, the server 100 may subtract 50 mg from 100 mg to calculate 50 mg as a daily serving amount of the vitamin C component.

In some cases, all of the fourth base dose of a certain nutritional component or cartridge is subtracted by the change amount information having a negative (−) value, and thus the daily serving amount of the nutritional component or cartridge may be calculated as 0 (zero).

Here, as an example, the daily serving amount information may be calculated as a unit value for the nutritional component of the dosage forms held for each cartridge. However, the present invention is not necessarily limited thereto, and the daily serving amount information may be calculated as a weight (e.g., mg) value for the nutritional component or cartridge.

In this way, since the server 100 of the present invention calculates the daily serving amount information by including the current condition information according to the change in the body condition of the user in the base dose information for each nutritional component or each cartridge according to the personal health information of the user in real time, the server 100 may provide information for each nutritional component or each cartridge more accurately tailored to the real-time health condition of the user.

In the step of converting to the number of dosage forms (S134), the server 100 may convert the daily serving amount for each nutritional component or each cartridge for a predetermined period into the number of dosage forms to be dispensed for each cartridge.

More specifically, in the step of converting to the number of dosage forms (S134), the server 100 may calculate first dosage form composition information by converting the daily serving amount of the first period into the number of dosage forms to be dispensed for each cartridge and calculate the second dosage form composition information by converting the daily serving amount of the second period into the number of dosage forms to be dispensed for each cartridge.

In the step of converting to the number of dosage forms (S134), the server 100 may convert the daily serving amount for each nutritional component or each cartridge into the number of dosage forms to be dispensed for each cartridge with reference to individual unit weight of the dosage form held in each cartridge and individual unit content of nutritional components contained in individual unit dosage form from the inquired cartridge information.

For example, if the calculated daily serving amount for vitamin B component is 330 mg, and as a result of the server 100 querying information of the vitamin B cartridge among the cartridge information, the individual unit weight of the vitamin B dosage form is 40 mg and the individual unit content of the vitamin B component is 30 mg, the server 100 may calculate 11 dosage forms obtained by dividing 330 mg by 30 mg as the number of vitamin B dosage forms to be dispensed from the vitamin B cartridge.

In this way, in the step of converting to the number of dosage forms of the present invention, when the server 100 converts the daily serving amount for each nutritional component or each cartridge into the number of dosage forms to be dispensed for each cartridge, the server 100 may convert the number of dosage forms based on the individual unit content of the nutritional component substantially contained in the individual unit dosage form.

Accordingly, the present invention can provide a more precise dose of the dosage form to the user.

For reference, if only the daily serving amount for the vitamin B component calculated under the same conditions as described above is calculated as 335 mg, the server 100 divides 330 mg by 30 mg and then the remaining 5 mg may be subjected to an integer-making process through rounding without being included in the conversion of the number of dosage forms when converting to the number of dosage forms.

Accordingly, even when the daily serving amount of the vitamin B component is 335 mg, the server 100 may calculate 11 dosage forms as the number of vitamin B dosage forms to be dispensed from the vitamin B cartridge. Accordingly, the present invention can prevent the user from overdosing on nutritional components.

So far, the case where the server 100 reflects the current condition information in the base dose information has been described as an example, but the present invention is not limited thereto, and in some cases, a method of reflecting the current condition information in the base dose information may be performed by the device 200.

To this end, a program or algorithm for reflecting the current condition information may be stored in the device 200 as well.

If the device 200 reflects the current condition information in the fourth base dose information, the device 200 may receive the third base dose information and the fourth base dose information from the server 100 before obtaining the current condition information from the user.

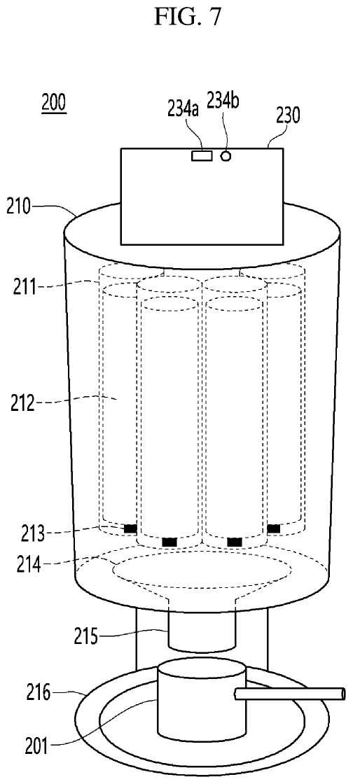

FIG. 7 is a diagram for explaining an example of a physical structure of the device 200 illustrated in FIG. 1.

In an embodiment of the present invention, the device 200 may be configured in a form in which the device terminal 230 is integrally coupled to hardware 210 for dispensing dosage forms. However, the device terminal 230 may also be provided in a form that is attachable to and detachable from the hardware 210. When the device terminal 230 is provided in an attachable and detachable form, the device terminal 230 may communicate with the dispensing hardware 210 wirelessly.

The dispensing hardware 210 may control the dispensing of the dosage form from the plurality of cartridges 212 storing the dosage form according to the dosage form composition information, and count the number of dispensed dosage forms.

The device terminal 230, for example, may obtain current condition information from the user and transmits it server 100, and receive dosage form composition to the information from the server 100 and transmit it to the dispensing hardware 210. However, the present invention is not limited thereto, and in some cases, it is also possible for the device terminal 230 to generate dosage form composition information and transmit it to the dispensing hardware 210.

As illustrated in FIG. 7, the hardware 210 according to an example of the present invention may include a cartridge insertion unit 211, a plurality of cartridges 212, a dosage form dispensing sensor 213, a dosage form dispensing passage 214, a dispensing port 215, and a cup holder 216. In addition, the hardware 210 may further include components of general electronic devices such as a power unit such as a dispensing device that dispenses dosage forms and an electric motor, a hardware communication unit, a camera unit, an external communication means, and a power supply unit.

A plurality of cartridges 212 may be inserted into each of the plurality of cartridge insertion units 211. Dosage forms containing different nutritional components may be stored in the plurality of cartridges 212.

In addition, two or more dosage forms may be mixed and stored in each cartridge 212. For example, the cartridge 212 storing a multivitamin dosage form in which a vitamin B component and a vitamin C component are mixed may be inserted.

Also, the dosage form dispensing passage 214 may be formed below the cartridge insertion unit 211. The dosage form dispensing passage 214 may be in communication with the dosage form dispensing port 215 of each of the plurality of cartridges 212. Various types of dosage forms dispensed from the plurality of cartridges 212 may be dispensed to the outside of the device 200 through the final dispensing port 215 through the dosage form dispensing passage 214. The cup holder 216 may be formed below the final dispensing port 215.

According to an embodiment of the present invention, the dosage form dispensing sensor 213 for detecting a dispensing amount of dosage form (i.e., the number of dispensed dosage forms) may be provided at the bottom of each of the plurality of cartridges 212. The dosage form dispensing sensor 213 may detect the number of dosage forms dispensed from each cartridge 212 and generate dispensing amount information, and the device 200 may store dispensing amount information and output or transmit dispensing result information to the server 100. For example, a photo interrupter, an ultrasonic sensor, an infrared sensor, a camera sensor, or the like may be applied to the dosage form dispensing sensor 213.

As illustrated in FIG. 7, the dosage form dispensing passage 214 may have a structure inclined toward the final dispensing port 215, and the dosage form dispensed from the cartridge 212 may roll toward the final dispensing port 215 and be collected in the cup 201 located on the cup holder 216.

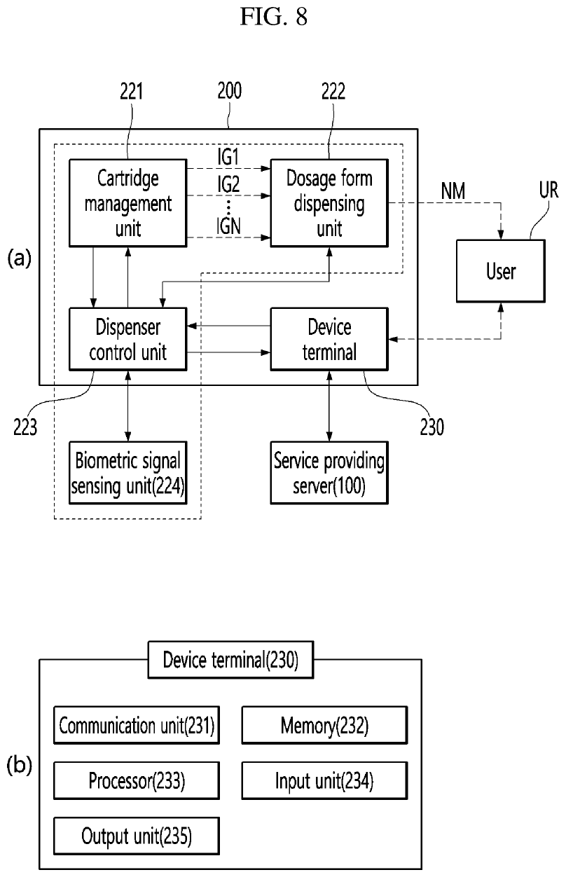

The device terminal 230 may be provided on an outer portion (e.g., upper portion) of the device 200, and the device terminal 230 may provide information to the user and receive data and commands from the user. For example, the device terminal 230 may receive or obtain data related to the health of the user, such as current condition information. The configuration of the device terminal 230 will be described in detail with reference to (b) of FIG. 8.

The device terminal 230 may communicate with the server 100 to transmit and receive information. Specifically, the device terminal 230 may receive dosage form composition information from the server 100 and transmit it to the hardware 210, and the hardware 210 may generate a control signal corresponding to the dosage form composition information to control the dosage form to be dispensed.

The (a) of FIG. 8 is a diagram for explaining an example of the circuit configuration of the device 200 illustrated in FIG. 7, and (b) of FIG. 8 is a diagram for explaining an example of a configuration of a device terminal.

As illustrated in (a) of FIG. 8, the circuit configuration of the device 200 according to an example of the present invention may include a cartridge management unit 221, a dosage form dispensing unit 222, a dispenser control unit 223, a biometric signal sensing unit 224, and a device terminal 230. Here, the cartridge management unit 221, the dosage form dispensing unit 222, the dispenser control unit 223, and the biometric signal sensing unit 224 may be provided in the dispensing hardware 210, and the biometric signal sensing unit 224 may be omitted in some cases.

The cartridge management unit 221 may store and manage cartridge information about the cartridges 212 in which dosage forms having various nutritional components are stored.

Specifically, a memory chip may be provided on each of the plurality of cartridges 212. The cartridge management unit 221 may receive, when the cartridge 212 storing a specific dosage form is inserted into the cartridge insertion unit 211, cartridge information including at least one of the type or name of the corresponding dosage form according to the nutritional component, individual unit weight of the dosage form, individual unit content of the nutritional component included in the dosage form, stored quantity information, manufacturing date, and manufacturer from the memory 120 provided for each cartridge 212, and transmit the cartridge information to the server 100 through the dispenser control unit 223 and the device terminal 230.

In addition, the cartridge management unit 221 may monitor dispensing amount information of the dosage forms (IG1 to IGN) dispensed from each cartridge 212 and remaining amount information of dosage forms stored in each cartridge 212, and the dispensing amount information and the remaining amount information may be output through the device terminal 230 or transmitted to the server 100 and the user terminal 300.

The dosage form dispensing unit 222 may include a dispensing device coupled to each cartridge 212 to dispense the dosage form, a drive motor mechanically coupled to each dispensing device to operate the dispensing device, and the dosage form dispensing sensor 213 provided at the bottom of each dispensing device to detect the number of dispensed dosage forms.

Each of the plurality of drive motors may operate the dispensing device of each of the plurality of cartridges 212 according to a control signal input from the dispenser control unit 223 to dispense the dosage form from each cartridge 212. The dosage form dispensing sensor 213 counts the number of dosage forms dispensed from the cartridge 212 to generate dispensing amount information, and the dispensing amount information may be provided to the dispenser control unit 223.

When the dosage form dispensing unit 222 is driven, the dosage form dispensing sensor 213 detects and counts the number of dosage forms dispensed from each cartridge 212, and the dispensing amount information generated by being counted may be transmitted to the device terminal 230 through the dispenser control unit 223. Then, the device terminal 230 may transmit the dispensing amount information to the server 100.

The dispenser control unit 223 may include a printed circuit board (PCB) on which a processor, memory, and communication module for controlling overall operations of the device 200 are mounted.

The dispenser control unit 223 may be electrically connected to the device terminal 230, the cartridge management unit 221, and the dosage form dispensing unit 222, and in some cases, the biometric signal sensing unit 224 may be connected to the device 200. The dispenser control unit 223 may control the cartridge management unit 221 and the dosage form dispensing unit 222 according to information provided from the device terminal 230 and provide information input from the cartridge management unit 221 and the dosage form dispensing unit 222 to the device terminal 230.

Specifically, the dispenser control unit 223 may provide cartridge information provided from the cartridge manager 221 to the device terminal 230. The dispenser control unit 223 may convert the dosage form composition information input from the device terminal 230 into a control signal, and may input a control signal to the dosage form dispensing unit 222 to control the dosage form to be dispensed from each cartridge 212.

In addition, the dispenser control unit 223 may provide the dispensing amount information provided from the dosage form dispensing unit 222 to the device terminal 230 and the cartridge management unit 221, the cartridge management unit 221 may generate remaining amount information by subtracting dispensing amount information from storage quantity information of the dosage form and track the remaining amount information of the dosage form for each cartridge 212, and provide the remaining amount information to the device terminal 230 through the dispenser control unit 223.

The biometric signal sensing unit 224 that senses a biometric signal of the user may be connected to the dispenser control unit 223 in a wired manner or wirelessly.

The biometric signal sensing unit 224 may include, for example, a biometric sensor and a sensor for detecting a biometric signal of user (e.g., electromyogram, brainwave, etc.), and the biometric signal obtained from the user may be provided to the device terminal 230 through the dispenser control unit 223.

As illustrated in (b) of FIG. 8, the device terminal 230 may include a communication unit 231, a memory 232, a processor 233, an input unit 234 and an output unit 235. The communication unit 231 of the device terminal 230 may communicate with the dispensing hardware 210 and the server 100.

The input unit 234 of the device terminal 230 may include various types of input units 234 for receiving a user's command, for example, a touch pad, a keyboard, a microphone (243b in FIG. 7), a camera (243a in FIG. 7), etc., and may further include various types of sensors (e.g., a touch sensor and a motion sensor) for detecting various types of external physical energy of the device 200.

When the camera (243a in FIG. 7) is provided as the input unit 234 of the device terminal 230, the camera 243a may photograph the movement of the user during a period after a time point at which the dosage form is dispensed. The device 200 may store and transmit photographing information of the user to the server 100 or transmit it to the medical institution server 500. In this case, the server 100 or the medical institution server 500 may perform image analysis or video analysis on the photographing information to accurately check whether or not the user actually ingested the dosage form dispensed from the device 200.

Alternatively, the device terminal 230 may further include the biometric sensor configured to detect biometric information (e.g., fingerprint, face, iris, vein, voice, etc.) of the user and a sensor that detects biometric signal (e.g., electromyogram, brain wave, etc.) of the user.

The output unit 235 of the device terminal 230 may include, for example, a display device, a speaker, etc. for providing information to the user. The output unit 235 may include, for example, at least one type of display device among types such as a liquid crystal display (LCD), a light emitting diode (LED), an organic light emitting diode (OLED), and a quantum dot light emitting diode (QLED).

The device terminal 230 may display information (e.g., the remaining amount information of dosage form remaining in the cartridge 212) related to the dispensing hardware 210, icon information for specifying the user, current condition information of the user, dispensing amount information, etc. through the output unit 235.

For example, the device terminal 230 may include a touch screen module capable of inputting and outputting as one module, and may receive information from the user through the touch screen module.

In addition, when the device terminal 230 includes the microphone (243b in FIG. 7) and a speaker, it is also possible for the device terminal 230 to perform a medical examination-by-interview for the user using a microphone and a speaker. For example, the device terminal 230 may output medical examination-by-interview inquiry information to the user through the speaker, receive an answer of the user through the microphone, and generate examination-by-interview result information.

In addition, it is also possible for the device terminal 230 to receive current condition information as voice input from the user using the microphone and the speaker. In this case, the device terminal 230 may generate a face image by photographing the face of the user using the camera. Thereafter, in the present invention, it is also possible to analyze the voice and facial image of the user and use it as the current condition information of the user.

The memory 232 of the device terminal 230 may store an application program for operating the dispensing hardware 210 in interlocked with the server 100 and an application program for obtaining current condition information from a user or in interlocked with the server 100, etc., and information received from the dispensing hardware 210 or information received from the server 100.

The processor 233 of the device terminal 230 may control to obtain current condition information from the user at a specific time point, and control the dosage form dispensing unit 222 to dispense a combination of dosage forms corresponding to the specific time point based on dosage form composition information reflecting the current condition information at the specific time point.

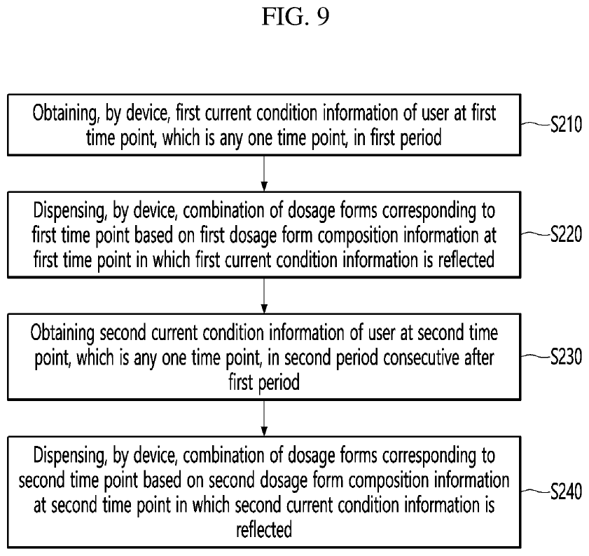

An example of an operating method of the device 200 by the processor 233 will be described in more detail with reference to FIG. 9.

FIG. 9 is a diagram for explaining an example of the operating method of the device 200 illustrated in FIG. 7.

In FIG. 9 and subsequent drawings, detailed descriptions of the contents overlapping with the previously described contents are omitted and replaced with the above contents.

As illustrated in FIG. 9, the operating method of the device 200 according to an example of the present invention may include the steps of obtaining first current condition information (S210), dispensing a combination of dosage forms corresponding to the first time point (S220), obtaining second current condition information (S230), and dispensing a combination of dosage forms corresponding to the second time point (S240).

In the step of obtaining the first current condition information (S210), the device 200 may obtain first current condition information of the user at the first time point during a first period.

In the step of dispensing the combination of dosage forms corresponding to the first time point (S220), the device 200 may dispense the combination of dosage forms corresponding to the first time point based on first dosage form composition information in which the first current condition information is reflected.

In the step of obtaining the second current condition information (S230), the device 200 may obtain second current condition information of the user at the second time point during a second period consecutive after the first period.

In the step of dispensing the combination of dosage forms corresponding to the second time point (S240), the device 200 may dispense the combination of the dosage forms corresponding to the second time point based on second dosage form composition information in which the second current condition information is reflected.

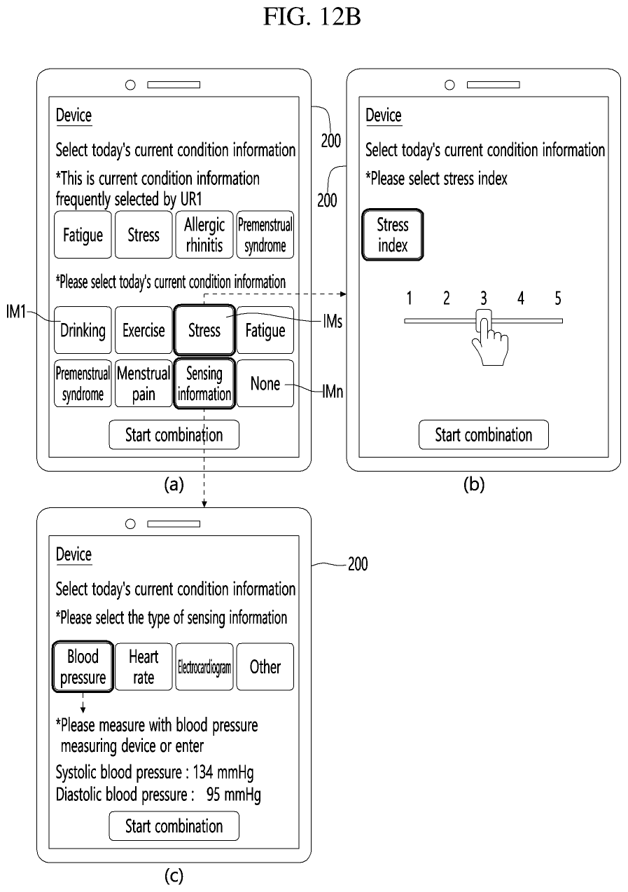

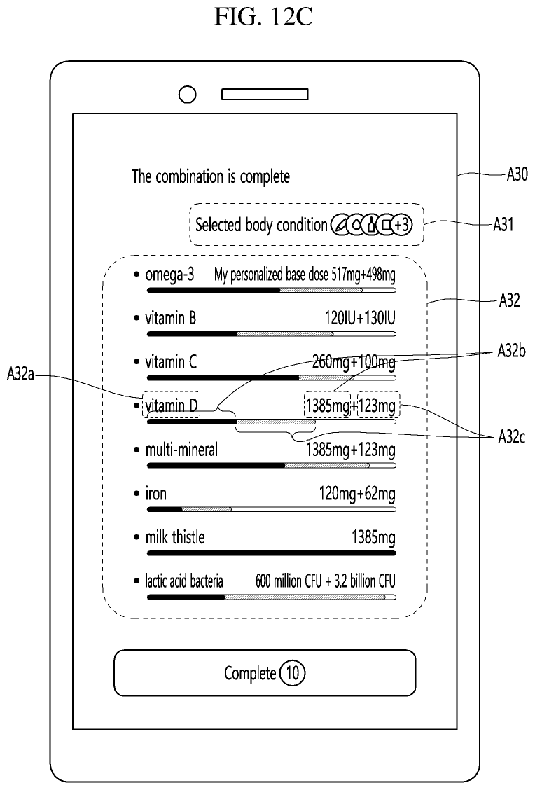

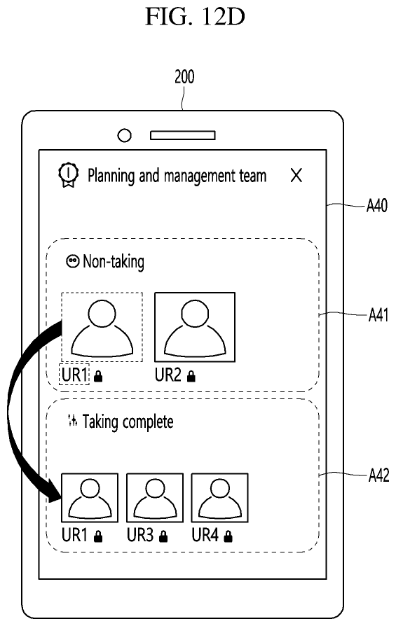

In the steps of obtaining the first and second current condition information (S210 and S230), the device 200 may respectively receive or obtain the first and second current condition information from the user through the input unit 234 or the biometric signal sensing unit 224 provided in the device terminal 230. For example, when the device terminal 230 includes the touch screen module, the device 200 may receive first and second current condition information from the user through the touch screen module. This will be described in more detail with reference to FIG. 12B.

However, the present invention is not limited thereto, the device 200 may collect and obtain the face image or voice information of the user through a camera or microphone provided in the device terminal 230 and use the information as the first and second current condition information.

Here, when the second current condition information is different from the first current condition information, a combination of dosage forms dispensed by the device 200 corresponding to the second time point may be different from a combination of dosage forms dispensed corresponding to the first time point.

That is, when the second current condition information is different from the first current condition information, at least one of the type and number of dosage forms dispensed by the device 200 corresponding to the second time point may be different from at least one of the type and number of dosage forms derived corresponding to the first time point.

In contrast, when the second current condition information is the same as the first current condition information, the combination of dosage forms dispensed by the device 200 corresponding to the second time point may be the same as the of combination dosage forms dispensed corresponding to the first time point.

That is, when the second current condition information is the same as the first current condition information, at least one of the type and number of dosage forms dispensed by the device 200 corresponding to the second time point may be the same as the type and number of dosage forms dispensed corresponding to the first time point.

To this end, each of the first and second dosage form composition information in which the first and second state information is respectively reflected in the base dose information for each nutritional component may include information about the type and number of dosage forms to be dispensed.

Accordingly, when the first and second current condition information are different from each other, at least one of the type and number of dosage forms included in the first and second dosage form composition information may be different from each other.

That is, when the second current condition information is different from the first current condition information, information about at least one of the type and number of dosage forms included in the second dosage form composition information may be different from information about at least one of the type and number of dosage forms included in the first dosage form composition information.

For example, when the plurality of cartridges 212 provided in the device 200 include first, second, and third dosage forms containing the first, second, and third nutritional components, the first and second dosage form composition information may include information about the first, second, and third dosage forms.

Here, it is assumed that the first and second current condition information are different from each other, the first dosage form containing the first nutritional component is related to the first current condition information, the second dosage form containing the second nutritional component is related to the second current condition information, and the third dosage form containing the third nutritional component is not related to the first and second current condition information.

In such a case, the first dosage form composition information includes information about the number of first dosage forms calculated as a daily serving amount by including the change amount according to the first current condition information in the base dose of the first nutritional component, and the second and third dosage forms may include information about the number of second and third dosage forms calculated as a daily serving amount with the base dose of the second and third nutritional components unchanged without a change amount according to the first current condition information.

In addition, the second dosage form composition information includes information about the number of second dosage forms calculated as a daily serving amount by including the change amount according to the second current condition information in the base dose of the second nutritional component, and the first and third dosage forms may include information about the number of first and third dosage forms calculated as a daily serving amount with the base dose of the first and third nutritional components unchanged without a change amount according to the second current condition information.

Therefore, the first and second dosage forms that are respectively related to the first and second current condition information may be different from each other in the number dispensed at the first and second time points. The number of the third dosage forms that are not related to the first and second current condition information may be the same in the number dispensed at the first and second time points.

In this way, the device 200 according to an example of the present invention may obtain current condition information from the user at the time point at which the user intends to take the dosage form, and provide the type and number (or dose) of the dosage form more optimized for the current health condition of the user.

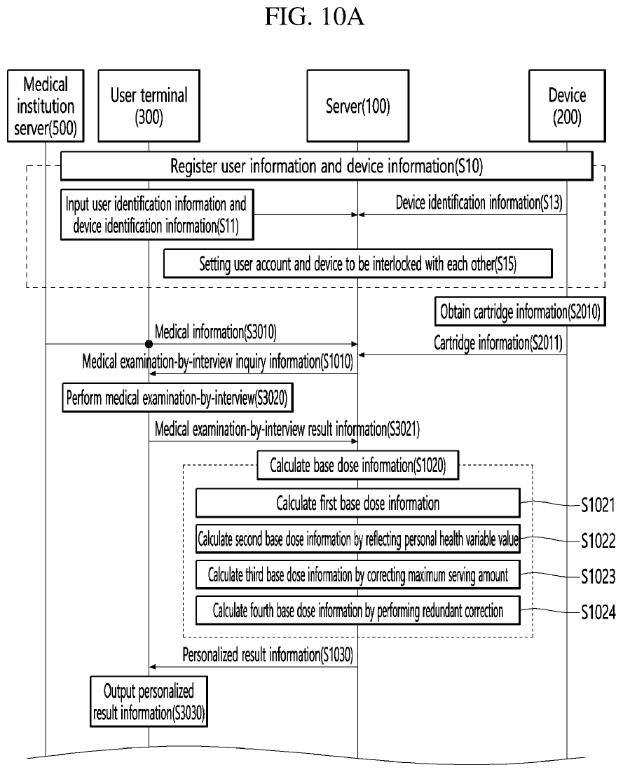

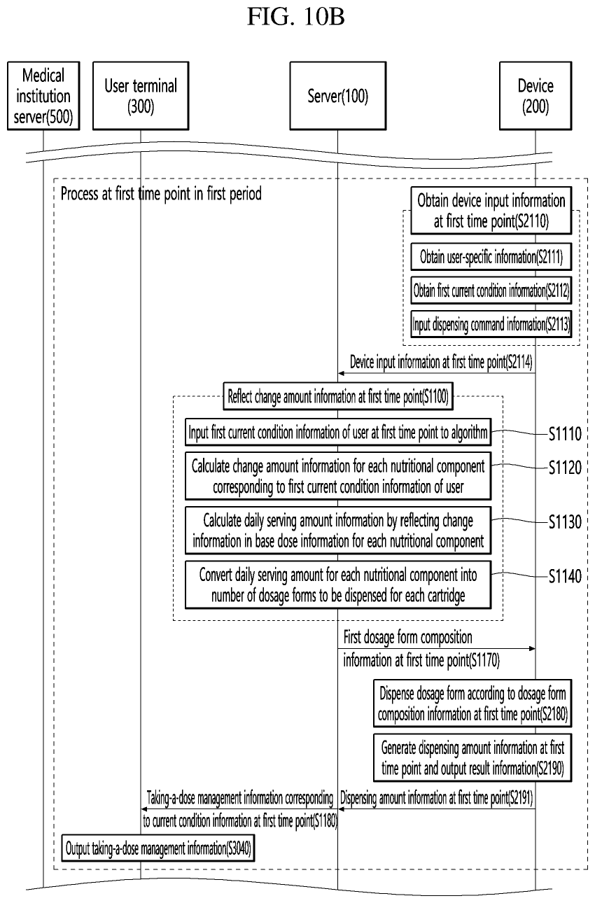

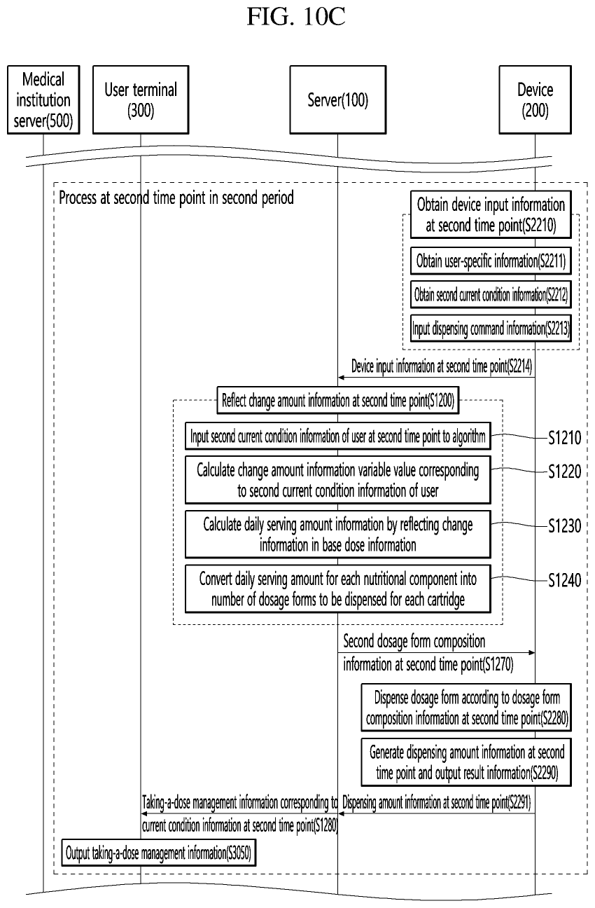

Hereinafter, various embodiments and modified examples in which the server 100, device 200, and user terminal 300 described above are operated in interlocked with each other will be described. In FIG. 10A and subsequent drawings, the detailed description of the content overlapping with the content described in FIGS. 1 to 9 is replaced with the previous content.

In addition, the order related to the flow and operation of information illustrated in FIG. 10A and subsequent drawings is only shown for explanation by way of example, and the present invention is not limited to the order related to the flow and operation of information.

FIGS. 10A to 10C are diagrams for explaining a first embodiment of a method in which the server, the user terminal, and the device illustrated in FIG. 1 operate together.

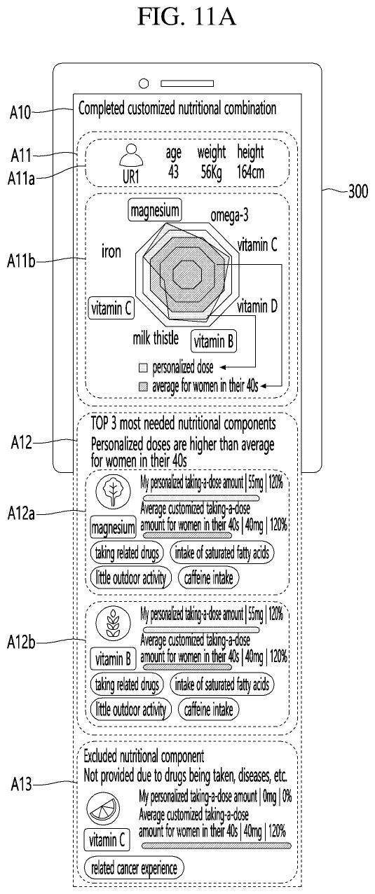

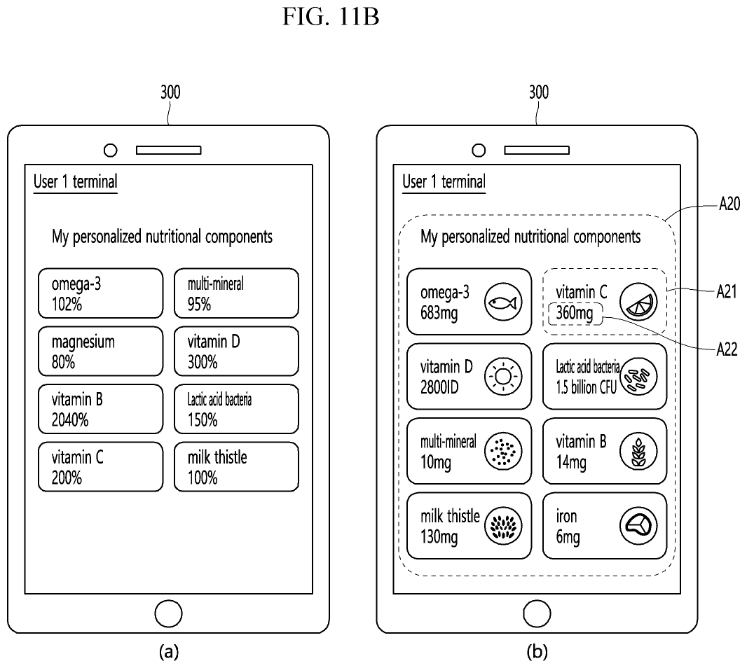

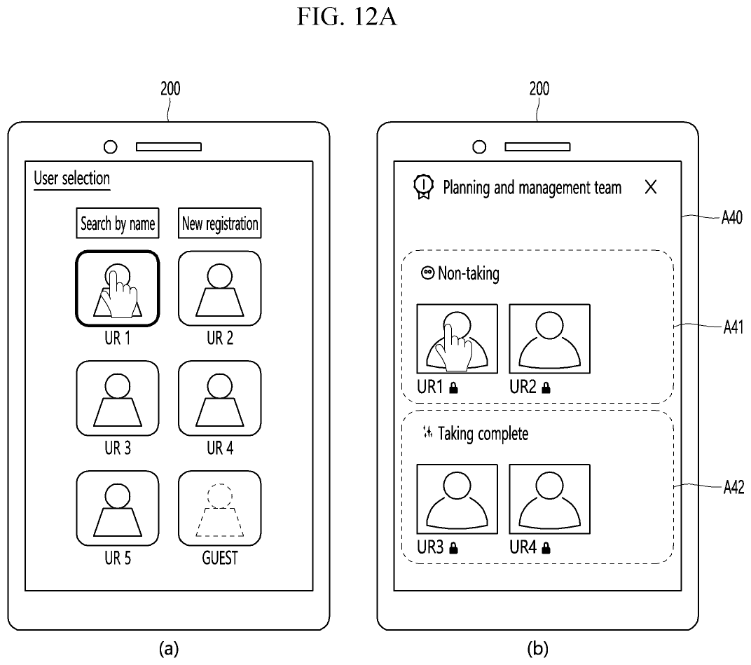

FIGS. 11A and 11B are diagrams for explaining an example of outputting personalized result information by the user terminal in FIG. 10A.

FIGS. 12A and 12B are diagrams for explaining an example of a method for a device to obtain device input information at the first and second time points of the first and second periods, respectively, in FIG. 10B or FIG. 10C.

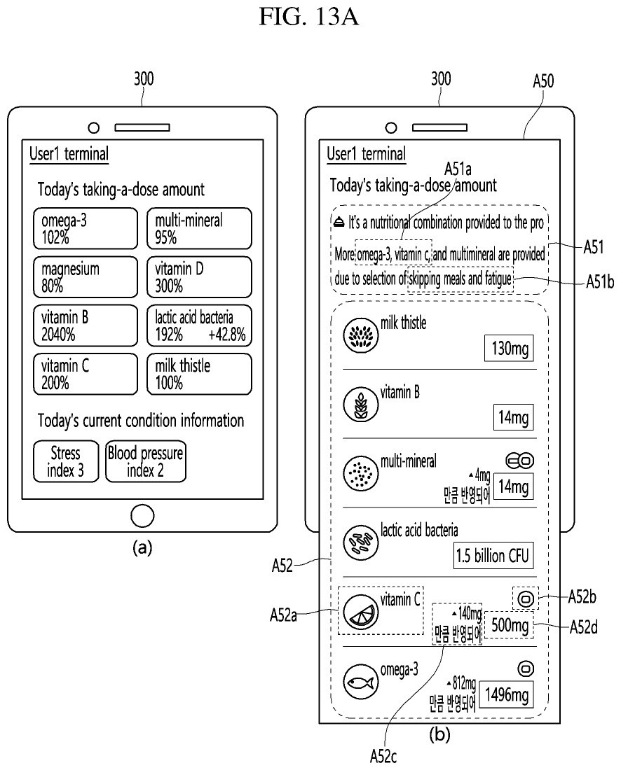

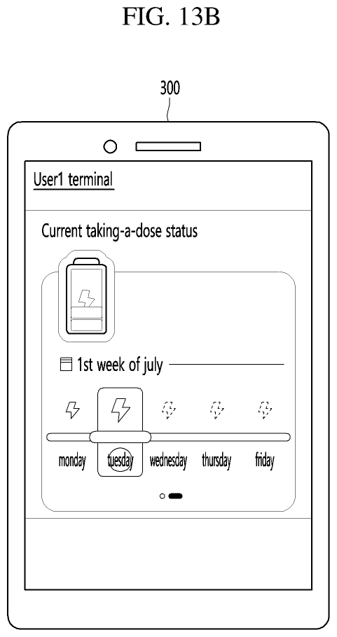

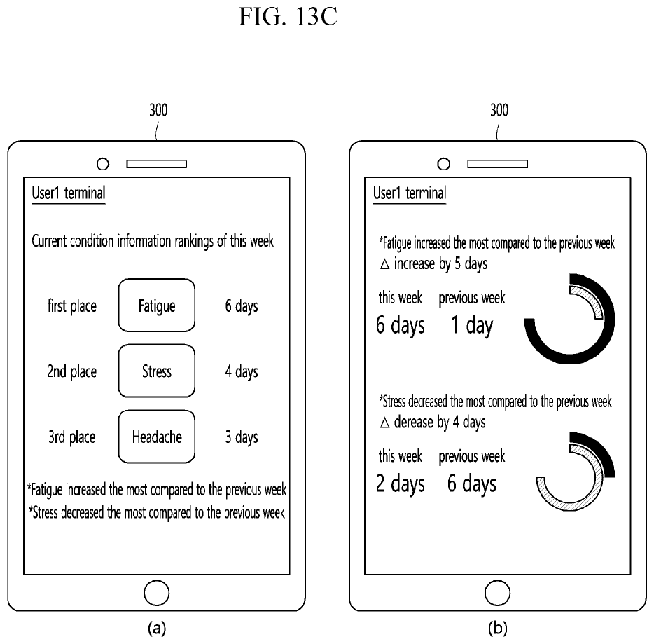

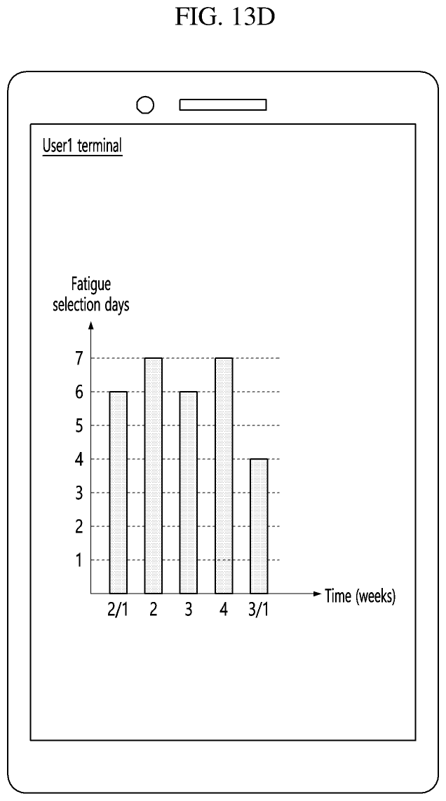

FIGS. 12C and 12D are diagrams for explaining an example of dispensing result information output by the device at the first and second time points of the first and second periods of FIG. 10B or FIG. 10C, respectively. FIGS. 13A to 13D are diagrams for explaining an example of taking-a-dose management information from output the user terminal corresponding to current condition information at the first and second time points of the first and second periods of FIG. 10B or FIG. 10C, respectively.

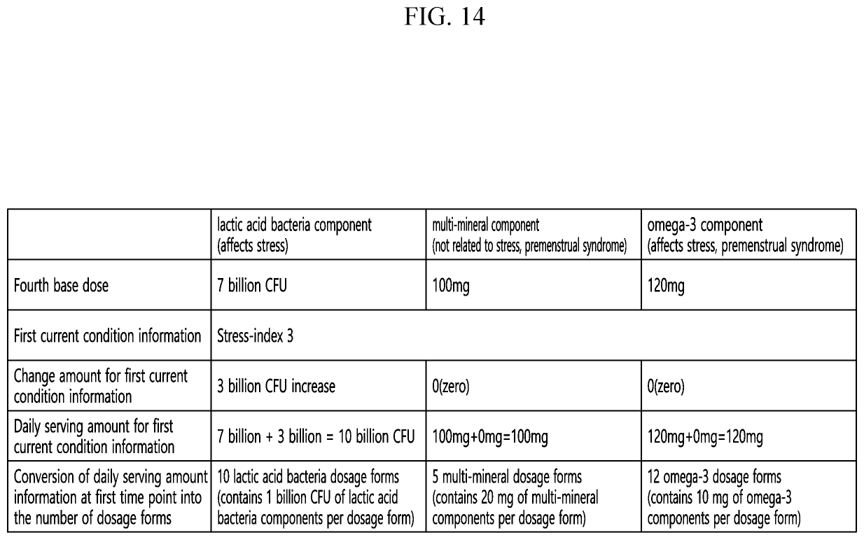

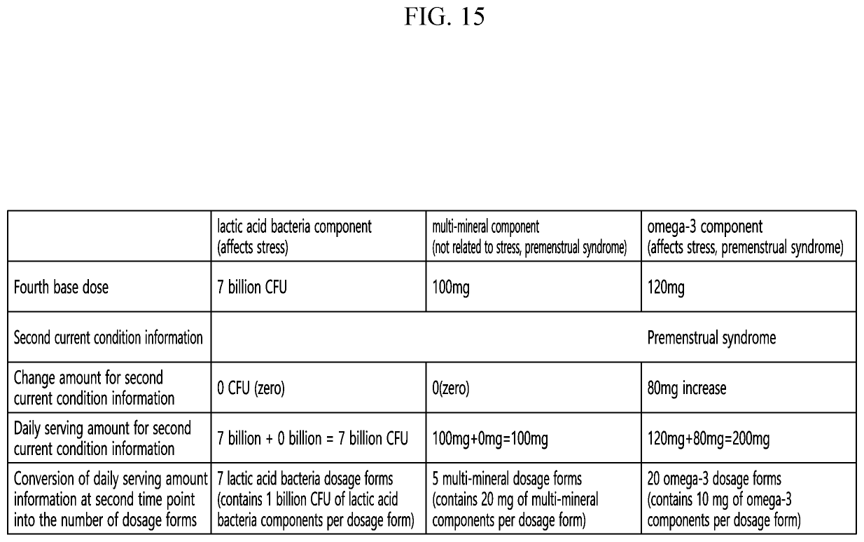

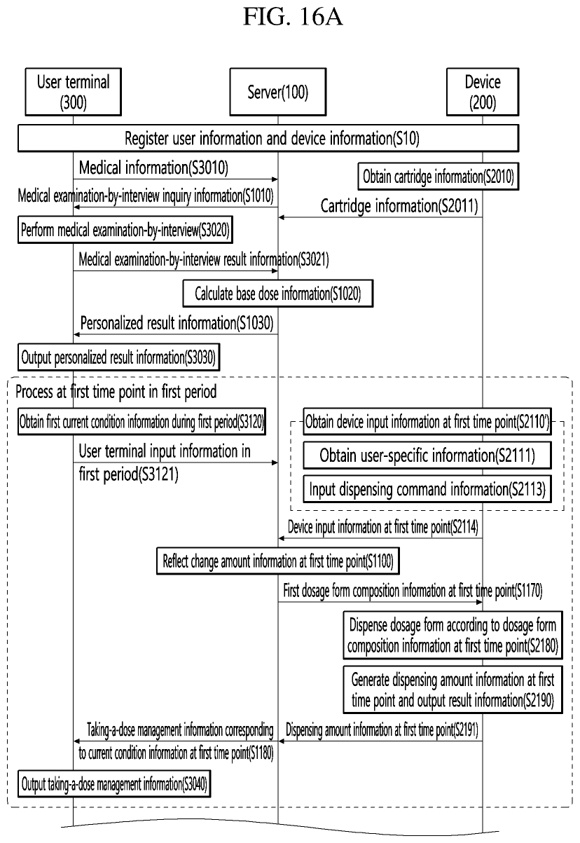

FIG. 14 is a table for explaining an example in which change amount information at the first time point is reflected, and FIG. 15 is a diagram for explaining an example in which change amount information at the second time point is reflected.

As illustrated in FIG. 10A, in the method in which the devices are operated in interlocked with each other according to the first embodiment of the present invention, a step of registering user information and device information (S10) may be performed.

In the step of registering user information and device information (S10), in order to register for a service provided by the server 100, the user terminal 300 may receive user identification information (e.g., user's name, phone number, email address, etc.) and device identification information (e.g., a unique code number of the device 200) from the user and transmit them to the server 100 (S11).

In addition, the device 200 may transmit the device identification information to the server 100 in order to register for the service provided by the server 100 (S13).

The server 100 may receive the user identification information and the device identification information from the user terminal 300 and receive the device identification information from the device 200. The server 100 may perform the step of registering the information (S10) by registering a user account in the server 100 using the user identification information and setting the user's account and the device 200 to be interlocked with each other (S15) using the device identification information.

When the step of registering the information (S10) is performed in this way, the server 100 may transmit the user account information (e.g., the user's name, nickname or contact interlocked with the device 200 to the device 200, and the device 200 may store the user account information therein. Thereafter, when the user intends to receive the dosage form through the device 200, the device 200 may use the user account information in order to obtain user specific information.

Thereafter, the server 100 may request medical information of the user from the medical institution server 500 through the user terminal 300. The server 100 may receive medical information of the user from the medical institution server 500 through the user terminal 300 (S3010). As another example, the server 100 may directly receive the medical information of the user from the medical institution server 500.

In addition, the server 100 may transmit (S1010) medical examination-by-interview inquiry information to the user terminal 300 at the request of the user. Here, a detailed description of the structure of the medical examination-by-interview inquiry information is replaced with the content described in FIG. 4.

The user terminal 300 may perform a medical examination-by-interview for the user according to the medical examination-by-interview inquiry information (S3020), receive an answer to each medical examination-by-interview inquiry information from the user, generate medical examination-by-interview result information, and transmit the generate medical examination-by-interview result information to the server 100 (S3021).

When performing the medical examination-by-interview, the user terminal 300 may display the medical examination-by-interview inquiry information through a touch screen module provided in the user terminal 300 and may receive an answer to the medical examination-by-interview inquiry information from the user. Alternatively, the user terminal 300 may output the medical examination-by-interview inquiry information as voice through a speaker and receive an answer from the user through a microphone.

In addition, when the plurality of cartridges 212 storing dosage forms of different nutritional components are mounted on the device 200, the device 200 may obtain cartridge information from each of the plurality of cartridges 212 (S2010) and transmit the information to the server 100 (S2011). The server 100 may receive cartridge information and use the cartridge information to calculate fourth base dose information.

The server 100 may calculate base dose information for each nutritional component optimized for the personal health condition of the user from personal health information of the user. Here, the personal health condition of the user may include at least one of user identification information, medical examination-by-interview result information, and medical information, and a detailed description thereof will be replaced with the content described above.

In the step of calculating the base dose information (S1020), the server 100, for example, may calculate first base dose information (S1021), calculate second base dose information by reflecting a personal health variable value extracted from the personal health information (S1022), calculate the third base dose information by performing at least one of the maximum serving amount correction and the minimum serving amount correction (S1023), and perform redundant correction to calculate fourth base dose information (S1024). Here, the method of calculating the first, second, third, and fourth base dose information is replaced with the content described in FIG. 4.

Thereafter, the server 100 may transmit personalized result information to the user terminal 300 (S1030). The personalized result information may include result report information for performing the medical examination-by-interview (S3020) and personalized base dose information. In addition, although not illustrated in the drawing, the server 100 may transmit personalized basic capacity information to the device 200.

The personalized base dose information may be provided to the user terminal 300 periodically or at the request of the user after the base dose information calculation step (S1020), and may be provided regardless of a specific event time point such as the first time point or the second time point. Alternatively, the personalized base dose information may be provided to the user terminal 300 when the base dose information is updated in the server.

The user terminal 100 may receive and output the personalized result information (S3030). As a more specific example, the user terminal may output result report information A10 for performing the medical examination-by-interview (S3020) as one of the personalized result information, as illustrated in FIG. 11A.

The result report information A10 may include a summary information area A11 and detailed information areas A12 and A13, as illustrated in FIG. 11A.

The summary information area A11 may include an information area for the user and a personalized area. In the information area for the user, a name (UR1), age (e.g., 43 years old), weight (e.g., 56 kg), height (e.g., 164 cm), gender (e.g., female), etc. of the users may be displayed. The personalized area may be displayed in the form of a graph in which an average required dose for each nutritional component for the same age group (e.g., 40's) and gender (e.g., female) as the user is compared with a personalized base dose for each nutritional component customized to the user. That is, as illustrated in A of FIG. 11, the average required dose and the personalized base dose for each nutritional component, such as magnesium, iron, and vitamin C, may be compared and displayed in the form of a polygonal graph.

The detailed information areas A12 and A13 may include a first detailed information area A12 and a second detailed information area A13.

Detailed information A12a and A12b about a plurality of nutritional components may be displayed in the first detailed information area A12.

In each of the detailed information A12a and A12b, the type of the corresponding nutritional component (e.g., magnesium), the personalized base dose value (e.g., 40 mg) for the corresponding nutritional component (e.g., 55 mg), the average required dose value for the same age group (e.g., 40's) and gender (e.g., female) as the user, and information that affects the personalized base dose of the nutritional component (e.g., related drug intake, saturated fatty acid intake, little outdoor activity, caffeine intake) may be displayed.

Here, the information affecting the personalized base dose includes personal health variable information of the user that affects the determination of the base dose of the corresponding nutritional component. For example, personal health variable information for at least one of (1) physical information of the user, (2) a drug that the user takes, (3) nutrients that the user takes, (4) lifestyle, (5) an illness or disease that the user has or has a history of having, and (6) a symptom that the user has or has a history of having may be included.

In the second detailed information area A13, nutritional components prohibited or restricted from being taken by the user may be displayed. More specifically, in the second detailed information area (A13), the type of nutritional component prohibited from being taken (e.g., vitamin C), the personalized base dose value for the corresponding nutritional component (e.g., 0 mg), the average required dose value (e.g., 40 mg) for the same age group (e.g., 40's) and gender (e.g., female) as the user, and information that affects the personalized base dose of the nutritional component (e.g., related cancer experience) may be displayed.

In addition, as illustrated in FIG. 11B, the user terminal may output personalized base dose information A20 for each nutritional component as one of the personalized result information. The personalized base dose information for each nutritional component may be output in various forms, as illustrated in (a) or (b) of FIG. 11B.

As illustrated in (a) or (b) of FIG. 11B, the user terminal may output the base dose information for each nutritional component as personalized base dose information.

The personalized base dose information A20 for each nutritional component may include a type of nutritional component A21 and a personalized base dose value A22. As illustrated in (a) of FIG. 11B, the personalized base dose compared to the minimum recommended amount for each nutritional component may be expressed as a percentage, or as illustrated in (b) of FIG. 11B, the base dose value for each nutritional component may be displayed in units of the corresponding nutritional component.

The type of nutritional component displayed here may be the same as the type of nutritional component stored in the cartridge of the device, but the present invention is not necessarily limited thereto. In the present invention, FIGS. 11A and 11B are separately described as examples for the personalized base dose for each nutritional component, but the output forms of FIGS. 11A and 11B may be mixed with each other. For example, when a specific nutritional component is selected by the user in (a) or (b) of FIG. 11B, detailed information of FIG. 11A may be displayed for the corresponding nutritional component.

Hereinafter, as illustrated in the tables of FIGS. 14 and 15, a case in which 7 billion CFU of lactic acid bacteria component, 100 mg of multi-mineral component, and 120 mg of Omega-3 component are calculated as base dose information (e.g., fourth base dose information) calculated by the server 100 based on the personal health information of the user will be described as an example.

Next, as illustrated in FIG. 10B, after the server 100 calculates the base dose information for the user, at the first time point during the first period, the device 200 may perform an operation of obtaining device input information at the first time point (S2110).

Here, the first time point of the first period may be a time point temporally separated from the time point at which the server 100 calculates the base dose information. That is, after the step of calculating, by the server 100, the base dose information is finished, the device 200 may perform the step of obtaining the device input information at the first time (S2110).

The step of obtaining the device input information at the first time (S2110) may include the steps of obtaining user specific information (S2111), obtaining first current condition information (S2112), and receiving dispensing command information (S2113).

Here, the first time point may mean a time point from when the user starts operating through the device 200 during the first period in order to take the dosage form to when the device 200 specifies the user and completes obtainment of the dispensing command information.

In the step of obtaining the user specific information (S2111), the device 200 may obtain specific information of the user who intends to take the dosage form through the device 200 at the first time point.

To this end, as illustrated in (a) of FIG. 12A, for example, the device 200 may display a user registered in the device 200 through the device terminal 230 as an icon. In this case, the device terminal 230 may display a name of the user (e.g., User 1, User 2, etc.) at the bottom of the icon using user identification information.

Alternatively, as illustrated in (b) of 12A, for example, the device 200 may display a current taking-a-dose status of the users based on the first time point of the first period on a screen A40 for specifying the user.

More specifically, the device 200 may distinguish and display a list of users A41 (e.g., UR1, UR2) who have not been provided with dosage forms and a list of users A42 (e.g., UR3, UR4) who have been provided with dosage forms based on the first time point of the first period. For example, in (b) of 12A, UR1 and UR2 may be users who have not been provided with dosage forms for the first period until the first time point, and UR3 and UR4 may be users who have been provided with dosage forms for the first period until the first time point.

The device 200 may obtain the user specific information from the user through the user interaction, such as touching an icon. In FIG. 12A, a case in which User 1 UR1 is specified as the user of the device 200 at the first time point is illustrated as an example.

In FIG. 12A, a case in which the device 200 receives an icon selected from the user and specifies the user as a method for specifying the user is illustrated as an example, but the present invention is not limited thereto. The device 200 may detect voice information of the user through a microphone provided in the device terminal 230 or obtain a facial image of the user through a camera to specify the user.

Thereafter, after the user is specified, the device 200 may display various examples of current condition information selectable by the user as icon images IM1 to IMn (drinking, exercise, etc.), as illustrated in (a) of FIG. 12B.

As illustrated in FIG. 10B, in the step of obtaining first current condition information (S2112), the device 200 may obtain the first current condition information from the user. To this end, as illustrated in (a) of FIG. 12B, the device 200 may display a plurality of icon images IM1 to IMn including an icon image for each of the first current condition information through the device terminal 230, and may obtain first current condition information corresponding to at least one icon IMs when the at least one icon IMs is selected from among the plurality of icon images through the user interaction.

Here, the first current condition information may mean current condition information obtained by the device 200 from the user corresponding to the first time point of the first period, and the first current condition information may include one or a plurality of pieces of information about body condition change of the user.

If the user selects stress IMs as the first current condition information as illustrated in (b) of FIG. 12B, the device 200 may provide an interface capable of selecting a stress index (or severity) representing a stress index to the user, and receive an input for the stress index (e.g., stress index 3) from the user.

In (a) of FIG. 12B, when the biometric signal sensing unit 224 of the user is connected to the device 200, the device 200 may display sensing information as an example of current condition information selectable by the user.

If the user selects sensing information as the first current condition information, as illustrated in (c) of FIG. 12B, the device 200 may provide an interface capable of selecting the type of sensing information (e.g., blood pressure, heart rate, electrocardiogram, etc.) to the user, and obtain the corresponding sensing information from the user through the biometric signal sensing unit 224 or directly receive the sensing information from the user.

Thereafter, the device 200 may receive dispensing command information from the user. For example, if the user selects a “start combination” button after the user completes the input of the first current condition information, the device 200 may recognize this as the dispensing command information and transmit the device input information at the first time to the server 100.

Here, the device input information at the first time point may include the user specific information, first current condition information (e.g., stress, index 3), and dispensing command information obtained from the user.

When transmitting the device input information at the first time point to the server 100, for example, the device 200 may convert the user specific information, the first current condition information (e.g., stress, index 3), and the dispense command information into code information usable in the algorithm stored in the server 100 and transmit the code information (S1114).

The server 100 that receives the device input information at the first time point from the device 200 may perform a step of reflecting the change amount information at the first time point (S1100). In the step of reflecting the change amount information at the first time point (S1100), the server 100 may calculate the number of dosage forms to be dispensed for each cartridge by reflecting the first current condition information in the base dose information for each nutritional component or each cartridge calculated based on personal health information of the user specified in the device 200.

Specifically, in the step of reflecting the change amount information at the first time point (S1100), for example, the server 100 may input the first current condition information at the first time point to the algorithm (S1110), calculate change amount information for each nutritional component or each cartridge corresponding to the first current condition information of the user (S1120), calculate daily serving amount information at the first time point by reflecting the change amount information in the base dose information for each nutritional component or each cartridge (S1130), and convert the daily serving amount for each nutritional component or each cartridge into the number of dosage forms to be dispensed for each cartridge (S1140). The daily serving amount calculated in the step S1130 may be the daily serving amount for the first period based on the first time point. Here, since the method of reflecting the change amount information is the same as that previously described with reference to FIG. 6, the detailed description thereof is replaced with the description described above.

For example, it is assumed that the lactic acid bacteria component described in FIG. 14 affects only ‘stress’, the Omega-3 component affects ‘premenstrual syndrome’, and the multi-mineral component described in FIG. 14 has no effect on stress and premenstrual syndrome.

When the server 100 receives stress as the first current condition information, the server 100 may input the stress at the first time point into the algorithm (S1110), and calculate the change information for each nutritional component corresponding to stress as follows: is 3 billion CFU increase for lactic acid bacteria component, 0 mg (zero) for multi-mineral component, and 0 mg (zero) for Omega-3.

Thereafter, in the step of calculating the daily serving amount information at the first time point (S1130), the server 100 may calculate 10 billion CFU as a daily serving amount by including the change amount (3 billion CFU) according to the first current condition information in the base dose information (e.g., 7 billion CFU) of the lactic acid bacteria component, calculate 100 mg as a daily serving amount of the multi-mineral component by including 0 mg in 100 mg, and calculate 120 mg as a daily serving amount of the Omega-3 component by including 0 mg into 120 mg.

Thereafter, in the step of converting the daily serving amount information at the first time point into the number of dosage forms (S1140), when 1 billion CFU of lactic acid bacteria component is contained per lactic acid bacteria dosage form, 20 mg of multi-mineral component is contained per 1 multi-mineral dosage form, and 10 mg of Omega-3 component is contained per omega-3 dosage form, the server 100 may calculate 10 lactic acid bacteria dosage forms, 5 multi-mineral dosage forms, and 12 Omega-3 dosage forms as the first dosage form composition information.

Here, information the interaction or input by selection of the user other than sensing information among the first current condition information may be input in the form of a code usable for an algorithm, and may be directly usable for the algorithm used in the step of reflecting the change amount information.

However, when the sensing information is included in the first current condition information received by the server 100, in order to reflect the sensing information as the change amount information, the server 100 may compare sensing information (e.g., blood pressure information) of the user with corresponding information (e.g., blood pressure information) among the personal health information to calculate a value for the degree of state change, and input the calculated value to the algorithm that reflects the change amount information at the first time point.

For example, when the maximum blood pressure (or systolic blood pressure) is 124 mmHg and the minimum blood pressure (or diastolic blood pressure) is 84 mmHg in blood pressure information according to the personal health information of the user and as illustrated in FIG. 12 (c), the systolic blood pressure is 134 mmHg and the diastolic blood pressure is 95 mmHg in the blood pressure information according to the first current condition information, the server 100 may compare the blood pressure information of the personal health information with the blood pressure information of the first current condition information, calculate a value (e.g., blood pressure index 2) for the degree of change in blood pressure, and input the value to the algorithm.

The server 100 may store the received first current condition information of the user in the memory of the server 100 and continuously track the current condition information of the user.

Thereafter, as illustrated in FIG. 10B, the server 100 may transmit information about the number of dosage forms to be dispensed for each cartridge at the first time point to the device 200 as first dosage form composition information (S1170).

The device 200 may convert the information about the number of dosage forms for each cartridge received from the server 100 into a control signal for the hardware 210 of the device 200 based on the information, and dispense the dosage form for each cartridge according to the first dosage form composition information at the first time point (S2180).

The device 200 may generate dispensing amount information by detecting the number of dosage forms dispensed from each cartridge 212 corresponding to the first time point using the dosage form dispensing sensor 213, and the device 200 may output the dispensing result information through the output unit 235 of the device based on the dispensing amount information (S2190) after the dosage form dispensing is complete.

As illustrated in FIG. 12C, for example, in the dispensing result information A30 output by the device 200, body condition information A31, nutritional component list information A32 dispensed from the device 200, type of nutritional component A32a, personalized base dose information A32b, and change amount information A32c may be displayed.

The body condition information A31 may include current condition information about the current time point at which the dosage form is dispensed from the device. For example, when the current time point is the first time point in the first period, the first current condition information may be displayed as the body condition information A31.

The nutritional component list information A32 may include information on the types of nutritional components A32a dispensed by the device at the current time point, and in the nutritional component list information A32, personalized base dose information A32b and change amount information A32c for each type of nutritional component may be contrasted and displayed.

In addition, as illustrated in FIG. 12D, after the device 200 outputs the dispensing result information (S2192), the device 200 may update and display the current taking-a-dose status of the users. For example, when the first user UR1 receives the dispensed dosage form at the first time point in the first period, a location of the first user UR1 may be updated and displayed from a user list A41 for the users not provided with the dosage form to a user list A42 for the users provided with the dosage form.

In addition, the device 200 may transmit the generated dispensing amount information to the server 100 (S2191). If the device 200 is equipped with a camera, it is also possible to generate and transmit taking-a-dose information of the user to the server 100 by photographing, by the device 200, whether or not the user takes the dispensed dosage form.

The server 100 receive dispensing amount may information about the number of dosage forms dispensed for each cartridge corresponding to the first time point from the device 200, generate taking-a-dose management information by reflecting the dispensing amount information at the first time point and the first current condition information, and then store the taking-a-dose management information in the memory 232. Accordingly, the server 100 may continuously track the types of dosage forms provided to the user and the dose or number of dosage forms for each type.

Here, the taking-a-dose management information may be generated by basically reflecting the dispensing amount information dispensed in real time, but may be generated by selectively further reflecting at least one of current condition information, daily serving amount information, and base dose information.

In addition, the server 100 may transmit base dose information for the user, first current condition information at the first time point, daily serving amount information and dispensing amount information for the first period to the user terminal 300 as taking-a-dose management information corresponding to the current condition information at the first time point (S1180).

Thereafter, the user terminal 300 may output the taking-a-dose management information as illustrated in FIGS. 13A to 13D as an example.

For example, the user terminal 300 may output today's taking-a-dose amount information as one of taking-a-dose management information, as illustrated in (a) or (b) of FIG. 13A. The taking-a-dose amount information may be information about the dose of the dosage form dispensed for a first user up to the first time point of the first period.

For example, as illustrated in (a) of FIG. 13A, the user terminal 300 compares the dispensing amount information for each nutritional component or each cartridge corresponding to the first time point with the base dose information and provides it as information about today's taking-a-dose amount (e.g., lactic acid bacteria: +42.8%), and may provide the first current condition information as today's current condition information (e.g., stress index 3, blood pressure index 2).

In addition, as another example, as illustrated in (b) of FIG. 13A, the user terminal 300 may output today's taking-a-dose amount information A50 by dividing it into dispensing summary information A51 and dispensing detailed information A52.

In the dispensing summary information A51, current condition information A512a (e.g., skipping meals, fatigue) selected by the user based on the current time point (e.g., first time point of first period) and information about the nutritional component A51b (e.g., Omega-3, vitamin c, multi-mineral) whose dose is changed by the current condition information may be summarized and displayed.

In the detailed dispensing information A52, at least one of the type of nutritional component A52a, the current condition information A52b, the change amount A52c, and the dispensing dose value for each nutritional component A52d may be displayed. In (b) of FIG. 13A, a case in which the type of nutritional component A52a (e.g., vitamin C), current condition information A52b (e.g., skipping meals), change amount A52c (e.g., 140 mg increase), and dispensing dose for each nutritional component A52d (e.g., 500 mg) are all displayed is illustrated as an example.

The dispensing dose for each nutritional component A52d may be the dose for each nutritional component dispensed through the device at the first time point during the first period, or the total dose for each nutritional component dispensed through the device up to the first time point during the first period. As an example, in (b) of FIG. 13A, the dose (e.g., 500 mg) dispensed through the device at the first time point during the first period is illustrated with respect to the dispensing dose for each nutritional component.

The current condition information A52b and the change amount A52c be displayed may corresponding to the corresponding nutritional component when the dispensing dose of the specific nutritional component is changed by the current condition information A52b. The user can confirm that 500 mg of vitamin C was provided as a skipping meals by increasing the amount of vitamin C by 140 mg through (b) of FIG. 13A.

In addition, although not illustrated, the user terminal 300 may output the dispensing dose A52d for each nutritional component on a weekly, monthly, or yearly basis. For example, when a specific nutritional component is selected by the user in FIG. 13A, the user terminal 300 may output the dispensing dose A52d for each nutritional component for a specific nutritional component on a weekly, monthly or yearly basis to provide the user with tracking information about change in the dispensing dose A52d for each nutritional component on a weekly, monthly or yearly basis.

In addition, the user terminal 300 may output current taking-a-dose status information as one of taking-a-dose management information. The current taking-a-dose status information may be output on a weekly, monthly, or yearly basis, and whether or not the device has supplied the dosage form to the user on a specific period or on a specific date may be output. As an example, FIG. 13B illustrates a case in which the user terminal 300 outputs the current dosage status information on a weekly basis.

In addition, the user terminal 300 may output current condition information as one of the taking-a-dose management information. Specifically, the server 100 may continuously trace and track the current condition information of the user to provide current condition information with a high frequency of the user selection for each period through the user terminal 300. In (a) of FIG. 13C, the user terminal 300 is illustrated as indicating the current condition information ranking on a weekly basis, but this is exemplary. It is also possible to indicate the current condition information ranking may be shown based on other periods such as one month and one quarter.

In addition, the server 100 may continuously trace and track the current condition information of the user provide a change in the current condition information of the current period compared to the previous period through the user terminal 300. For example, the server 100 may provide the current condition information with the most increased or decreased selection frequency compared to the previous week through the user terminal 300. In (b) of FIG. 13C, the user terminal 300 is illustrated as indicating a change in current condition information on a weekly basis, but this is exemplary. It is also possible to indicate the changes in current condition information based on other periods such as one month or one quarter.

In addition, the server 100 may continuously trace and track the current condition information of the user to provide the number of selected days for each period for specific current condition information (e.g., fatigue) through the user terminal 300. In FIG. 13D, the user terminal 300 is illustrated as indicating the number of days selected for specific current condition information on a basic basis of one week, but this is exemplary. It is also possible to indicate the number of selected days based on other periods such as one month or one quarter

Thereafter, as illustrated in FIG. 10C, at the second time point during the second period, the device 200 may perform a step of obtaining device input information at the second time point (S2210). The method of obtaining device input information at the second time point may include the steps of receiving dispensing command information (S2213), obtaining user specific information (S2211), obtaining second current condition information (S2212), and receiving dispensing command information (S2213). The second time point may mean a time point from when the user starts operating through the device 200 during the second period for the user in order to take the dosage form to when the device 200 specifies the user and completes obtainment of the dispensing command information.

Since the method of obtaining device input information at the second time point is the same as the method of obtaining device input information at the first time point except that the time point is different, the detailed description is replaced with the description of FIGS. 12A and 12B.

At the second time point, the device 200 may obtain the same user (e.g., User 1) as the user at the first time point as user specific information, and obtain second current condition information that is the same as or different from the first current condition information. Hereinafter, a case in which premenstrual syndrome (PMS) different from the first current condition information (e.g., stress) is selected as the second current condition information will be described as an example.

The device 200 may transmit, as device input information at the second time, the user specific information, the second current condition information (e.g., premenstrual syndrome), and the dispensing command information to the server 100 (S2214).

The server 100 that receives the device input information at the second time point from the device 200 may perform a step of reflecting the change information at the second time point (S1200).

In the step of reflecting the change amount information at the second time point (S1200), the server 100 may specify a user based on the device input information of the second time point received from the device 200, and calculate the number of dosage forms to be dispensed for each cartridge by reflecting the second current condition information (e.g., premenstrual syndrome) in the base dose information for each nutritional component or each cartridge calculated based on the personal health information of the specified user (e.g., User 1).

In the step of reflecting the change amount information at the second time point (S1200), for example, the server 100 may input the second current condition information at the second time point to the algorithm (S1210), calculate change amount information for each nutritional component or each cartridge corresponding to the second current condition information of the user (e.g., premenstrual syndrome) (S1220), calculate the daily serving amount information at the second time point by reflecting the change amount information in the base dose information for each nutritional component or each cartridge (S1230), and convert the daily serving amount for each nutritional component or each cartridge into the number of dosage forms to be dispensed for each cartridge (S1240). Here, since the method of reflecting the change amount information is the same as that previously described with reference to FIG. 6, the detailed description is replaced with the description previously described.

For example, as illustrated in FIG. 15, when the server 100 receives premenstrual syndrome as the second current condition information, the server 100 may input the premenstrual syndrome at the second time point to the algorithm (S1110), and calculate the change information for each nutritional component or each cartridge corresponding to premenstrual syndrome as follows: 0 CFU (zero) for lactic acid bacteria component, 80 mg increase for Omega-3, and 0 mg (zero) for multi-mineral component change (S1220).

Thereafter, in the step of calculating the daily serving amount information at the second time point (S1230), the server 100 may calculate 200 mg as a daily serving amount by including the change amount (increased by 80 mg) according to the second current condition information (e.g., premenstrual syndrome) in the base dose information (e.g., 120 mg) of the Omega-3 component, calculate 7 billion CFU as a daily serving amount by including the change amount 0 CFU (zero) in 7 billion CFU for lactic acid bacteria, and calculate 100 mg as a daily serving amount by including 0 mg in 100 mg of the multi-mineral component.

Thereafter, in the step of converting the daily serving amount information at the second time point into the number of dosage forms (S1240), the server 100 may calculate 7 lactic acid bacteria dosage forms, 5 multi-mineral dosage forms, and 20 Omega-3 dosage forms as the second dosage form composition information.

Thereafter, as illustrated in FIG. 10C, the server 100 may transmit information about the number of dosage forms to be dispensed for each cartridge at the second time point to the device 200 as the second dosage form composition information (S1270).

The device 200 may dispense the dosage form for each cartridge according to the second dosage form composition information at the second time (S2280). The device 200 may generate dispensing amount information corresponding to the second time point and output dispensing result information (S2290). Here, the description of the dispensing result information is replaced with the previous description on FIGS. 12C to 12D.

In addition, the device 200 may transmit dispensing amount information at the second time point to the server 100 (S2291).

The server 100 may receive dispensing amount information corresponding to the second time point from the device 200, generate taking-a-dose management information based on the dispensing amount information, and then store the taking-a-dose management information in the memory 232. Accordingly, the server 100 may continuously track the types of dosage forms provided to the user and the dose or number of dosage forms for each type.

In addition, the server 100 may transmit the second current condition information, the daily serving amount information, and the dispensing amount information of the user at the second time point as the taking-a-dose management information corresponding to the current condition information at the second time point (S1280), and the user terminal 300 may output the taking-a-dose management information (S3050). Here, the description of the taking-a-dose management information is replaced with the previous description on FIGS. 13A to 13D.

In this way, the present invention may reflect current condition information, which is information about a change in the temporary body condition of the user, to the personal health information of the user.

For example, as described above, when the user had stress at the first time point of the first period, only the number of lactic acid bacteria dosage forms was increased (lactic acid bacteria dosage forms: 7->10) and provided by reflecting the stress in the base dose information (e.g., 7 lactic acid bacteria dosage forms, 5 multi-mineral dosage forms, 12 Omega-3 dosage forms). However, if the user has premenstrual syndrome at the second time point of the second period, only the number of Omega-3 dosage forms may be increased (Omega-3 dosage forms: 12->20) and provided.

Accordingly, the present invention can provide the nutritional components optimized and precisely tailored to the personal health information and temporary real-time current condition information of the user to the user.

In addition, in the present invention, by calculating the daily serving amount by reflecting the change amount according to the current condition information in the base dose information at the time point at which the user receives the dosage form through the device 200 in a state in which the base dose information is calculated and stored in advance, the dosage form can be quickly provided to the user, and thus user convenience can be greatly improved.

More specifically, when calculating the base dose information, which is personalized dose information, based on the personal health information of the user, there are many variables to be input and the amount of data to be processed is large, and thus it may take a relatively long time to calculate the personalized dose information (e.g., 5 to 20 minutes). Therefore, when the user calculates personalized taking-a-dose information for each nutritional component at the time point at which the user receives the dosage form through the device, the inconvenience that the user has to wait for a long time may be caused.

However, in the case of the present invention, since the base dose is calculated in advance and then the current condition information of the user is collected in real time each time the user receives the dosage form and reflected in the base dose information, and thus the processing speed of data can be reduced to within a few seconds (e.g., 0.1 to 3 seconds), and user convenience can be greatly improved.

In the first embodiment of the present invention, the case where the device 200 obtains the current condition information from the user has been described as an example, but the present invention is not limited thereto, and it is also possible for the user terminal 300 to obtain the current condition information from the user.

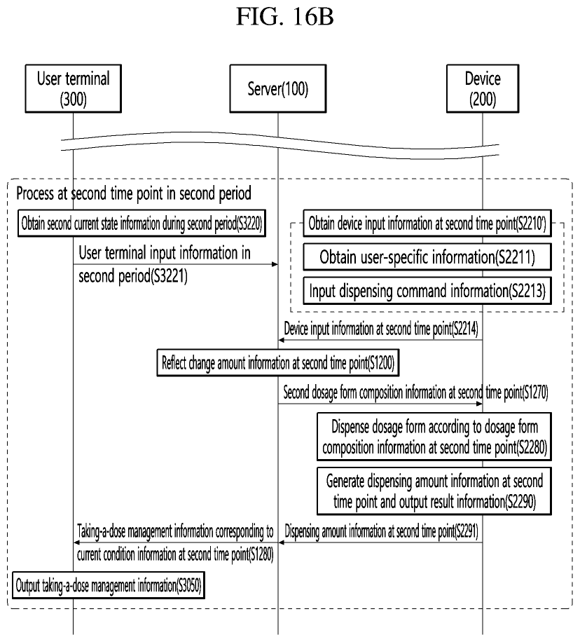

FIGS. 16A and 16B are diagrams for explaining a first modified example of the first embodiment described in FIGS. 10A to 10C.

For FIGS. 16A and 16B, descriptions of overlapping contents with those described with reference to FIGS. 10A to 15 are replaced with the previous descriptions, and other parts are mainly described.

As illustrated in FIG. 16A, in the first modified example of the first embodiment of the present invention, first, a step of registering user information and device information (S10) may be performed.

Next, the user terminal 300 may receive medical examination-by-interview inquiry information from the server 100 (S2010), perform a medical examination-by-interview (S3020), generate medical examination-by-interview result information, and transmits the generated medical examination-by-interview result information to the server 100 (S3021).

In addition, the server 100 may receive medical information from the user terminal 300 (S3010) and may receive cartridge information from the device 200 (S2011).

Thereafter, the server 100 may calculate base dose information for each nutritional component or each cartridge optimized for personal health condition of the user from personal health information including at least one of user identification information, medical examination-by-interview result information, and medical information (S1020). The server 100 may provide personalized result information to the user terminal based on the calculated base dose (S1030), and the user terminal 300 may output the personalized result information as illustrated in FIGS. 11A and 11B previously described.

Next, after the base dose information is calculated (S1020), during the first period, the user terminal 300 may obtain first current condition information from the user (S3120).

To this end, as described previously with reference to FIG. 12B, an application capable of executing an interface that can receive the current condition information from the user may be installed and stored in the user terminal 300 in advance.

The user terminal 300 may obtain first current condition information from the user for a period up to a first time point during the first period through the interface (S3120), and transmit the first current condition information to the server 100 as user terminal input information in the first period (S3121).

Although not illustrated, when the server 100 receives the first current condition information obtained by the user interaction from the user terminal 300 during the first period, the server 100 may transmit information indicating that the first current condition information has been received from the user terminal 300 to the device 200. In this case, when the device obtains the input information at the first time point (S2110′), it is also possible to display to the user through the application that there is the first current condition information obtained from the user terminal.

In the step of obtaining device input information at the first time point (S2110′), since the device 200 receives the first current condition information through the user terminal 300, the device 200 may receive only user specific information and dispensing command information from the user (S2111, S2113), excluding the current condition information, as device input information at the first time, and transmit the input information to the server 100 (S2114).

However, the present invention is not limited thereto. In the present invention, in order to find out whether there is first current condition information to be additionally input from the user, the device 200 may further perform the step of obtaining the current condition information as described previously with reference to FIG. 12B.

Thereafter, the server 100 may perform the step of reflecting the change amount information at the first time point (S1100). In the step of reflecting the change amount information at the first time point (S1100), the first current condition information received from the user terminal 300 may be input as an input variable to an algorithm for calculating the change amount, and change amount information at the first time point may be calculated to calculate the first dosage form composition information at the first time point.

Next, the device 200 may receive the first dosage form composition information from the server 100 (S1170), dispense the dosage form from each cartridge 212 according to the first dosage form composition information (S2180), generate dispensing amount information and outputs dispensing result information (S2190), and transmit the dispensing amount information at the first time point to the server 100 (S2191).

The server 100 may provide the received dispensing amount information at the first time point to the user terminal 300 as taking-a-dose management information corresponding to the current condition information at the first time point (S1180), and the user terminal 300 may output the taking-a-dose management information.

Thereafter, as illustrated in FIG. 16B, at the second time point during the second period, the user terminal 300 may obtain second current condition information from the user (S3220) and transmit the second current condition information to the server 100 as user terminal input information in the second period (S3221).

In a step of obtaining device input information at the second time point (S2210′), the device 200 may obtain, as the device input information, user specific information and dispensing command information excluding the second current condition information (S2211 and S2213) and transmit the information to the server 100 (S2214).

Next, in the step of reflecting the change amount information at the second time point (S1200), the server 100 may input the second current condition information received from the user terminal 300 as an input variable to the algorithm for calculating the change amount, reflect the change amount information at the second time point to calculate daily amount information, and calculate second dosage form composition information at the second time point.

The device 200 may receive the second dosage form composition information from the server 100 (S1270), dispense the dosage form from each cartridge 212 according to the second dosage form composition information (S2280), generate dispensing amount information and outputs dispensing result information (S2290), transmit the dispensing amount information at the second time point to the server 100 (S2291).

The server 100 may provide the dispensing amount information at the second time point received as the taking-a-dose management information corresponding to the current condition information at the second time point to the user terminal 300 (S1280), and the user terminal 300 may output the taking-a-dose management information (S3050).

When obtaining the first current condition information or the second current condition information through the user terminal 300, the first or second current condition information may be obtained through an input unit 234 provided in the user terminal 300, for example, a touch screen module, a microphone, or a camera.

When the first and second current condition information is received through the touch screen module of the user terminal 300, similar to the case of receiving the first and second current condition information through the device terminal 230, the user may receive the first and second current condition information by selecting an icon for the current condition information.

In addition, an image obtained by photographing a part of the body such as the user's face or an image obtained by photographing an analysis kit used by the user (e.g., urine kit) through the camera of the user terminal 300 may be obtained and used as the first and second current condition information.

Alternatively, when the first and second current condition information is received through the microphone of the user terminal 300, voice information of the user obtained through the microphone may be analyzed and used as the first and second current condition information.

In the first modified example of the first embodiment of the present invention, the user may input the first current condition information or the second current condition information of the user in advance through the user terminal 300 without going through the device 200, and receive a dosage form by inputting only user specific information and dispensing command information into the device 200 at a desired time, and thus the present invention can further increase user convenience.

So far, in the first embodiment of the present invention and the first modified example of the first embodiment, the case where the device 200 or the user terminal 300 obtains information selected by the user from the user through an interface as first current condition information or second current condition information has been described as an example.

However, the present invention is not limited thereto, and sensing information detected by the user terminal 300 or the external device 400, which is interlocked with the user terminal 300 to detect the health condition of the user, without being selected by the user may also be used as the first current condition information or second current condition information of the user. An explanation of this is as follows.

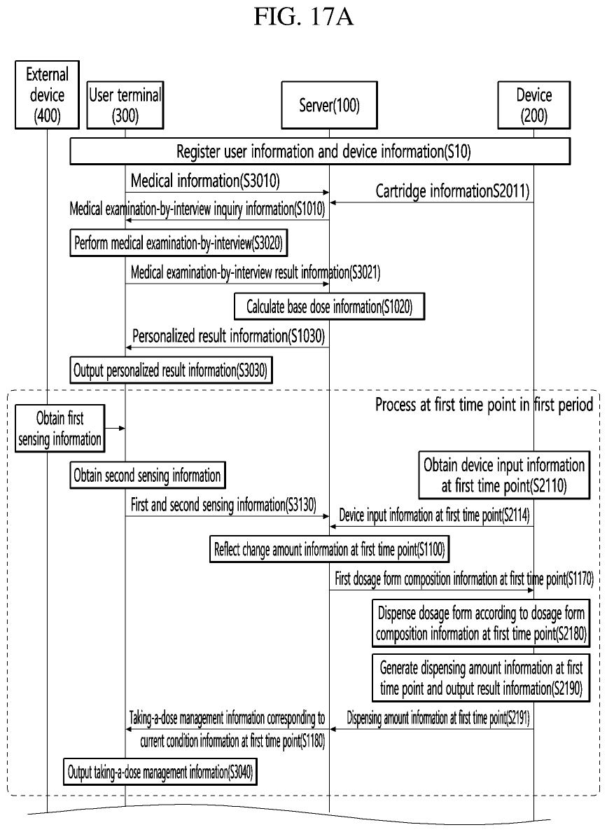

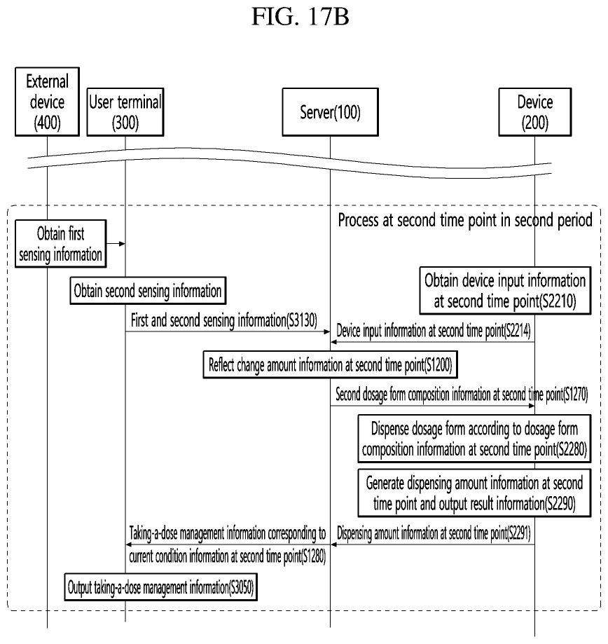

FIGS. 17A and 17B are diagrams for explaining a second modified example of the first embodiment described with reference to FIGS. 10A to 10C.

As in FIGS. 1 and 2 described previously, when the user terminal 300 includes the user terminal sensor 360 or the external device 400 that is interlocked with the user terminal 300 to collect health-related information for the user, as in FIGS. 17A and 17B, the server 100 may use the sensing information transmitted from the user terminal 300 or the external device 400 as the first current condition information or the second current condition information.

As illustrated in FIG. 17A, in the second modified example of the first embodiment of the present invention, the step of registering user information and device information (S10) may be performed first.

Next, the user terminal 300 receives the medical examination-by-interview inquiry information from the server 100 (S1010) and performs the medical examination-by-interview (S3020) to generate medical examination-by-interview result information, and transmit the generated medical examination-by-interview result information to the server 100 (S3021).

Thereafter, the server 100 may calculate the base dose information for each nutritional component or each cartridge optimized for the personal health condition of the user from personal health information including at least one of the user identification information, the medical examination-by-interview result information, and the medical information (S1020), and transmit personalized result information to the user terminal 300 based on the calculated base dose information. The user terminal 300 may output the personalized result information.

Next, after the base dose information is calculated, the external device 400 may detect a biometric signal from the body of the user and generate first sensing information during a period up to the first time point in the first period and transmit the first sensing information to the user terminal 300.

Alternatively, the user terminal sensor 360 provided in the user terminal 300 may detect the biometric signal of the user during the period up to the first time point in the first period to generate second sensing information.

Thereafter, the user terminal 300 may transmit the first sensing information or the second sensing information to the server 100 as the first current condition information (S3130). To this end, in the user terminal 300, an application that is interlocked with the external device 400 and an application capable of transmitting the first sensing information or the second sensing information to the server 100 as the first current condition information may be installed and stored.

Unlike the matters described above, the user terminal may also transmit the first sensing information received from the external device 400 to a third server that provides a service according to the function of the external device 400, and may receive analyzed or processed first sensing information from the third server and transmit the analyzed or processed first sensing information to the server 100 of the present invention.

Such first sensing information or second sensing information may be information generated by sensing the biometric signal from a body of the user, rather than information selected by selection of the user through an interaction operation of the user.

When the server 100 receives the first sensing information or the second sensing information as the first current condition information from the user terminal 300 during a period up to the first time point in the first period the server 100 may transmit information notifying that the first current condition information has been received to the device 200.

Thereafter, in the step of obtaining the device input information at the first time point (S2110), the device 200 may obtain device input information at the first time point from the user (S2110). In this case, since the device 200 has received the first current condition information through the user terminal 300, the device 200 may receive only user specific information and dispensing command information from the user and transmit the information to the server 100.

However, the present invention is not limited thereto. In the present invention, in order to find out whether or not there is current first condition information to be additionally input from the user, the device 200 may further perform the step of obtaining the current condition information as described previously with reference to FIGS. 10A to 12.

The server 100 may compare the first sensing information or the second sensing information received during the first period with the personal health information of the user to calculate a value for the degree of state change, and input the calculated value to an algorithm that reflects the change amount information at the first time point.

Thereafter, the server 100 may perform the step of reflecting the change amount information at the first time point (S1100). In the step of reflecting the change amount information at the first time point (S1100), a value according to the first sensing information or the second sensing information may be input as an input variable to an algorithm for calculating the change amount as the first current condition information.

However, the present invention is not limited thereto. In the present invention, when the device 200 obtains the current condition information and transmits it to the server 100 as in FIGS. 10A to 12 described previously, the server may reflect the current condition information obtained from the device together with the first and second sensing information when reflecting the change amount information at the first time point.

Accordingly, in the step of reflecting the change amount information at the first time point (S1100), change amount information at the first time point is calculated to calculate first dosage form composition information at the first time point, and the server may transmit the first dosage form composition information to the device (S1170).

Next, the device 200 may receive the first dosage form composition information from the server 100 and dispense the dosage form from each cartridge 212 according to the first dosage form composition information (S2180), generate dispensing amount information, output dispensing result information (S2190), and transmit the dispensing amount information at the first time point to the server 100 (S2191).

The server 100 may provide the dispensing amount information of the first time point, which is received as taking-a-dose management information corresponding to the current condition information at the first time point, to the user terminal 300 (S1180), and the user terminal 300 may output the taking-a-dose management information (S3050).

Thereafter, as illustrated in FIG. 17B, the server 100 may receive the first sensing information obtained by the external device 400 or the second sensing information obtained by the user terminal 300 during a period up to the second time point in the second period as second current condition information (S3230), and the server 100 may transmit information notifying that the second current condition information has been received to the device 200.

Thereafter, in the step of obtaining the device input information at the second time point (S2210), the device 200 may obtain device input information at the second time point. In this case, since the device 200 has received the second current condition information through the user terminal 300, the device 200 may receive only user specific information and dispensing command information from the user and transmit them to the server 100, but the present invention is not limited thereto.

The server 100 may compare the first sensing information or the second sensing information received during the second period with the personal health information of the user to calculate the value for the degree of state change, and input the calculated value to an algorithm that reflects the change amount information at the second time point.

Accordingly, in the step of reflecting the change amount information at the second time point (S1200), change amount information at the second time point is calculated to calculate first dosage form composition information at the first time point, and the server may transmit the first dosage form composition information to the device (S1270).

Next, the device 200 may receive the second dosage form composition information from the server 100, and dispense the dosage form from each cartridge 212 according to the second dosage form composition information (S2280), generate dispensing amount information and output dispensing result information (S2290), and transmit the dispensing amount information at the second time point to the server 100 (S2291).

The server 100 may provide the received dispensing amount information at the second time point as taking-a-dose management information corresponding to the current condition information at the second time point to the user terminal 300 (S2180), and output the taking-a-dose management information (S3050).

In the second modified example to the first embodiment of the present invention, even if the user does not take any interaction for inputting the first or second current condition information, the server 100 may receive the first or second current condition information of the user and the user simply inputs only user specific information and dispensing command information into the device 200 at a desired time to receive the dosage form. Therefore, the present invention can further increase user convenience.

In the first embodiment of the present invention and the first and second modified examples of the first embodiment so far, although the case where the medical examination-by-interview is performed in the user terminal 300 has been described as an example, the present invention is not necessarily limited thereto, and the medical examination-by-interview may also be performed in the device 200. An explanation of this is as follows.

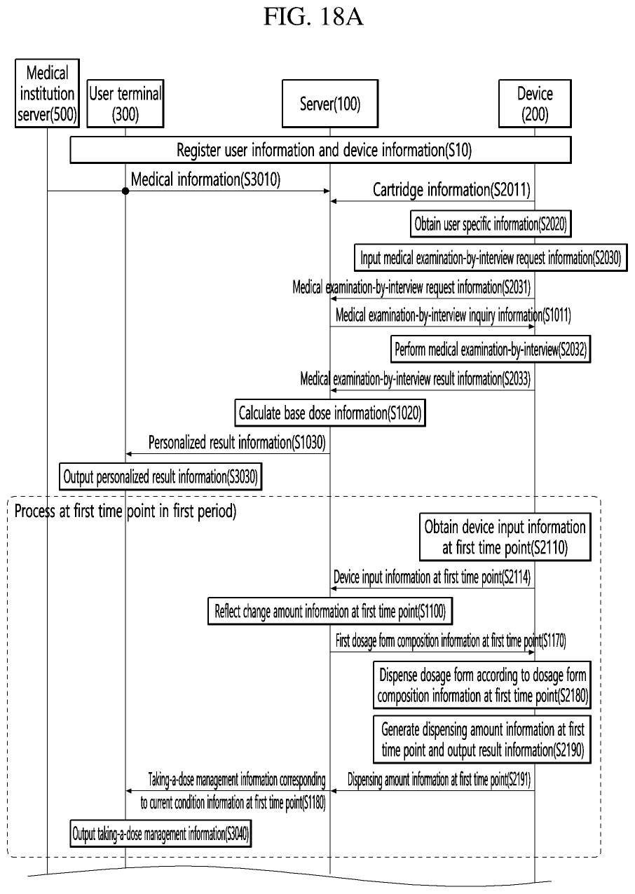

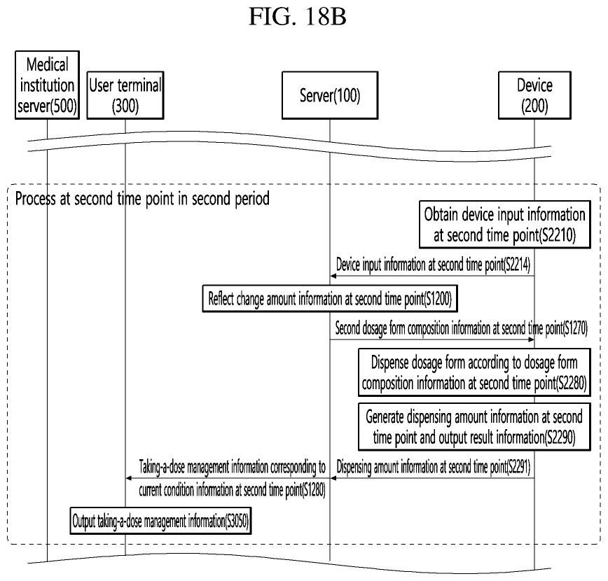

FIGS. 18A and 18B are diagrams for explaining a third modified example of the first embodiment described with reference to FIGS. 10A to 10C.

In FIGS. 18A and 18B, except for the contents related to performing the medical examination-by-interview, the description of the steps of registering user information and device information (S10), receiving medical information by the server 100 (S3010), receiving cartridge information by the server 100 (S2011), calculating base dose information by the server 100 (S1020), obtaining (S2110, S2210) and transmitting (S2114, S2214), by the device 200, device input information at the first and second time points, respectively, calculating and transmitting (S1170, S1270) first and second dosage form composition information, by the server 100, by reflecting the change amount information at the first and second time points by the server 100 (S1100, S1200), receiving the first and second dosage form composition information and dispensing the dosage form, by the device 200 (S2180, S2280), and generating the dispensing amount information and outputting the dispensing result information (S2190, S2290) by the device 200 and transmitting the information by the device 200 to the server 100, are the same as those described previously in the first embodiment, and thus the contents of this part are replaced with the contents of FIGS. 10A to 10C described previously.

As illustrated in FIG. 18A, unlike the examples described above, the medical examination-by-interview may also be performed in the device 200. In order to provide the convenience of medical examination-by-interview to the user, the device terminal 230 may be provided to be attachable to and detachable from the dispensing hardware 210 in the device 200. In this case, the device terminal 230 may perform wireless communication with the dispensing hardware 210.

In addition, an application for performing the medical examination-by-interview may be installed and stored in advance in the device terminal 230.

The device 200 may provide an interface for performing the medical examination-by-interview through the device terminal 230 and obtain the user specific information (S2020) from the user. Here, the method of obtaining the user specific information may be the same as that described previously with reference to FIG. 12A, for example, or information about a user (e.g., name, contact number, email, etc.) may be newly registered from the user.

Thereafter, when the device 200 receives information about a medical examination-by-interview request from the user through the device terminal 230 (S2030), the device 200 may transmit medical examination-by-interview request information indicating that the medical examination request by interview has been input from the user to the server 100 (S2031).

The server 100 may provide medical examination-by-interview inquiry information to the device terminal 230 in response to the medical examination-by-interview request information of the device terminal 230 (S1011), and the device terminal 230 may perform a medical examination-by-interview for the user according to the medical examination-by-interview inquiry information (S2032).

In this case, the device terminal 230 may output the medical examination-by-interview inquiry information as an image to the user through a touch screen module that is one of the output units 235 of the device 200, and receive a result value of the medical examination-by-interview through the interaction of the user and generate medical examination-by-interview result information.

Alternatively, the device terminal 230 may also provide medical examination-by-interview inquiry information to the user through the speaker, and receive the voice information of the user through a microphone and generate the medical examination-by-interview result information.

Thereafter, the device terminal 230 may transmit the generated medical examination-by-interview result information to the server 100 (S2033) so that the medical examination-by-interview result information is used in the step of calculating, by the server 100, the base dose information (S1020).

In the third modified example to the first embodiment of the present invention, by making it possible to perform the medical examination-by-interview for the user in the device terminal 230, it makes possible for the elderly people, children, visually impaired person, or hearing impaired person with less experience in using the user terminal 300 such as a smartphone to more easily perform the medical examination-by-interview.

Accordingly, in the present invention, a dosage form optimized for personal health information can be provided from the device 200 even to a user with less experience of using the user terminal 300, so that the usability of the device 200 can be further increased.

In the first embodiment and the first, second, and third modified examples to the first embodiment so far, although the case where the server 100 reflects the change amount information of the first and second time points is described as an example, the present invention is not limited thereto, and it is also possible for the device 200 to reflect the change amount information of the first or second time points in some cases. An explanation of this is as follows.

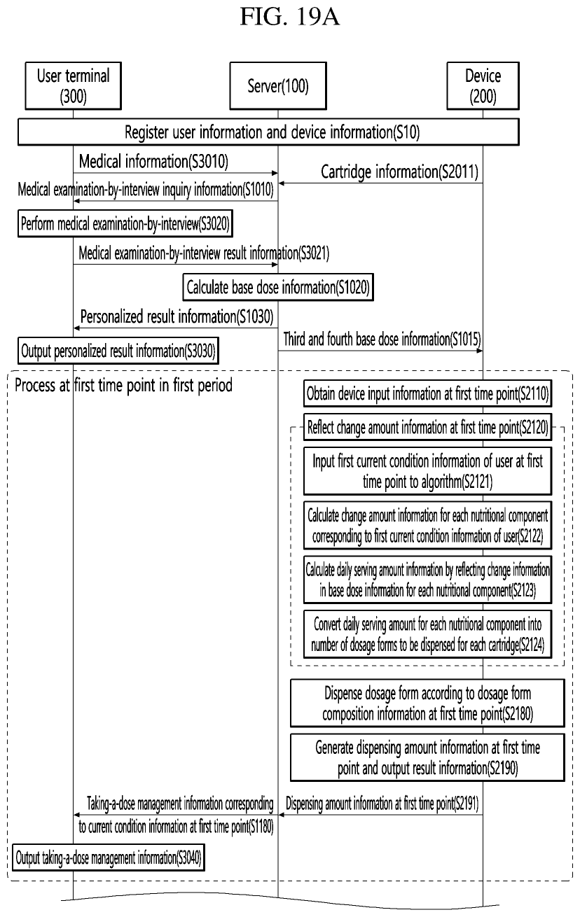

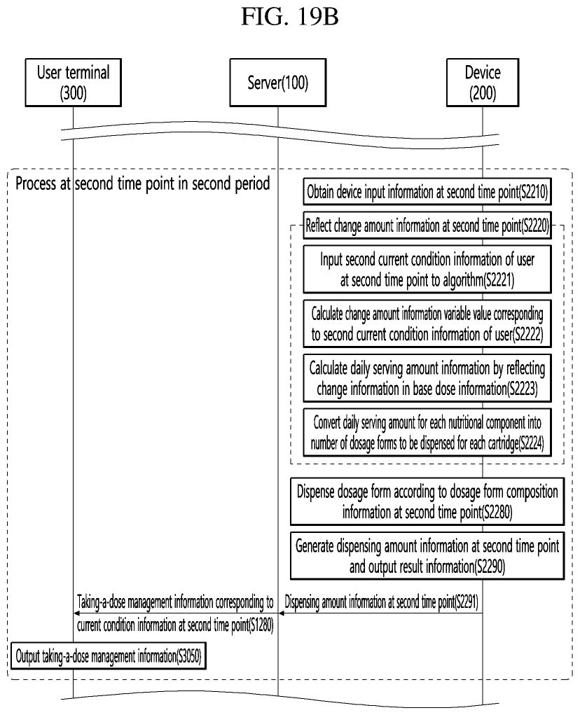

FIGS. 19A and 19B are diagrams for explaining a second embodiment of the method in which the service providing server 100, the user terminal 300, and the device 200 illustrated in FIG. 1 operate in interlocked with each other.

In FIGS. 19A and 19B, the description of the content overlapping with the content described in the first embodiment and various modified examples of the first embodiment is replaced with the description described previously, and the other parts are mainly described.

As illustrated in FIG. 19A, in the second embodiment of the present invention, first, the step of registering the user information and device information (S10) may be performed.

Next, the user terminal 300 may receive the medical examination-by-interview inquiry information from the server 100 (S1010) and performs the medical examination-by-interview (S3020) to generate the medical examination-by-interview result information, and transmit the generated medical examination-by-interview result information to the server 100 (S3021). In addition, the server 100 may receive medical information from the user terminal 300 (S3010), and receive cartridge information from the device 200 (S2011).

Thereafter, the server 100 may calculate base dose information for each nutritional component or each cartridge optimized for the personal health condition of the user from the personal health information including at least one of the user identification information, the medical examination-by-interview result information, or the medical information (S1020), and transmit personalized result information to the user terminal based on the base dose information (S1030). The user terminal 300 may output personalized result information as illustrated in FIGS. 11A and 11B described previously.

The server 100 may transmit third base dose information and fourth base dose information for each nutritional component or each cartridge for the user calculated in the step of calculating the base dose information (S1020) to the device 200 before the first period in order for the change information to be used for reflecting the base dose information by the device 200. Here, the server 100 may transmit the third base dose information and the fourth base dose information to a user registered as a user in the corresponding device 200 (S1015).

In the second embodiment, although the case where the third base dose information and the fourth base dose information are transmitted to the device 200 has been described as an example, the first base dose information or the second base dose information may be transmitted instead of the third base dose information and the fourth base dose information, and only one of the third base dose information and the fourth base dose information may be transmitted.

Thereafter, at the first time point during the first period, the device 200 may perform the step of obtaining the device input information from the user (S2110) to obtain the user specific information, the first current condition information, and the dispensing command information. Since this is the same as described in the first embodiment and the third modified example of the first embodiment described above, a detailed description thereof will be omitted.

Thereafter, the device 200 may perform the step of reflecting the change amount information at the first time point (S2120). To this end, an algorithm reflecting the change amount information may be stored and installed in the device 200 in advance. The method in which the device 200 reflects the change amount information may be substantially the same as that of FIG. 6 except that the subject is the device 200.

Therefore, in the step of reflecting, by the the device 200, the change amount information at the first time point (S2120), the device 200 may calculate first dosage form composition information at the first time point by reflecting the first current condition information.

To this end, in the step of reflecting the change amount information at the first time point (S2120), first, the device 200 may input the first current condition information of the user obtained at the first time point to an algorithm reflecting the first current condition information (S2121) to calculate change amount information for each nutritional component or each cartridge corresponding to the first current condition information of the user (S2122).

Here, when the device 200 calculates the change amount information for each nutritional component or each cartridge corresponding to the first current condition information, for example, the third base dose information received from the server 100 may be used.

Thereafter, after the device 200 calculates daily serving amount information for the first period by including the change amount according to the first current condition information in the fourth base dose information for each nutritional component or each cartridge (S2123), the device 200 may convert the daily serving amount for the first period into the number of dosage forms to be dispensed for each cartridge (S2124) to calculate first dosage form composition information.

Next, the device 200 may control the dosage form to be dispensed (S2180) according to the first dosage form composition information and the dispensing command information at the first time point, generate dispensing amount information corresponding to the first time point by counting the dosage form to be dispensed, and output dispensing result information (S2190).

Thereafter, the device 200 may transmit the dispensing amount information at the first time point to the server 100 (S2191), the server 100 may reflect the dispensing amount information at the first time point and transmit taking-a-dose management information corresponding to the first current condition information at the first time point to the user terminal 300 (S1180), and the user terminal 300 may output the taking-a-dose management information (S3050).

Thereafter, as illustrated in FIG. 19B, at the second time point during the second period, the device 200 may perform the step of obtaining device input information from the user (S2110) to obtain user specific information, second current condition information, and dispensing command information.

Thereafter, the device 200 may perform the step of reflecting the change amount information at the second time point (S2220) in order to calculate the second dosage form composition information at the second time point by reflecting the second current condition information.

In the step of reflecting the change amount information at the second time point (S2220), first, the device 200 may calculate change amount information for each nutritional component or each cartridge corresponding to the second current condition information of the user (S2222) by inputting the second current condition information of the user obtained at the second time point to an algorithm that reflects the information (S2221).

Thereafter, the device 200 may calculate daily serving amount information for the second period by including the change amount according to the second current condition information in the fourth base dose information for each nutritional component or each cartridge (S2223), and then, converts the daily serving amount for the second period into the number of dosage forms to be dispensed for each cartridge to calculate the second dosage form composition (S2224) information.

Next, the device 200 may control the dosage form to be dispensed according to the second dosage form composition information and the dispensing command information at the second time point (S2280), generate dispensing amount information corresponding to the second time point by counting the dispensed dosage form, and output dispensing result information (S2290).

Thereafter, the device 200 may transmit the dispensing amount information at the second time point to the server 100 (S2291), the server 100 may reflect the dispensing amount information at the second time point and transmit taking-a-dose management information corresponding to the second current condition information at the second time point to the user terminal 300 (S1280), and the user terminal 300 may output the taking-a-dose management information (S3050).

The second embodiment of the present invention may be implemented when communication between the device 200 and the server 100 is temporarily disconnected at the first time point in the first period or the second time point in the second period at which the user tries to receive the dosage form from the device 200 so that the device 200 is operated in offline mode or the device 200 needs to reflect the change amount information of the first or second time points.

In the second embodiment of the present invention, the case where the device 200 reflects the change amount information at the first or second time point has been described as an example, but the device 200 may also calculate the base dose information in addition to this. A more detailed description of this is as follows.

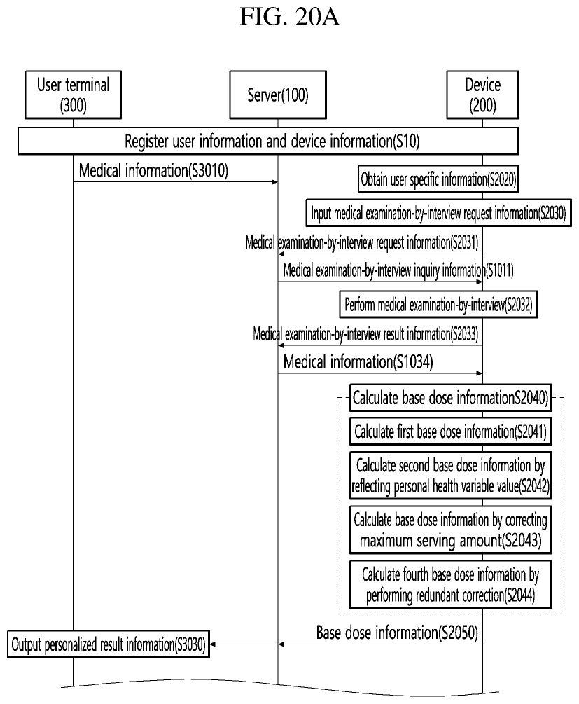

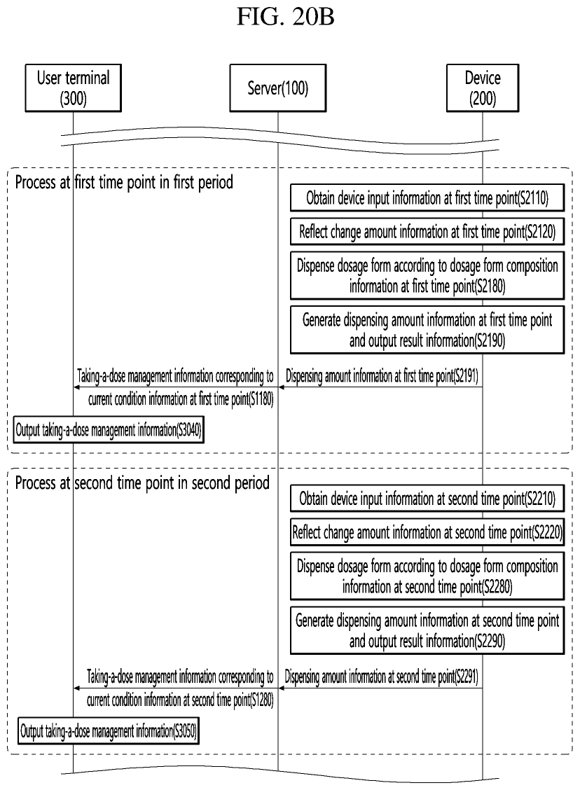

FIGS. 20A and 20B are diagrams for explaining a first modified example of the second embodiment described with reference to FIG. 19.

In FIGS. 20A and 20B, the description of the content overlapping with the content described in the first embodiment, its modified example, and the second embodiment is replaced with the previous descriptions, and other parts are mainly described.

As illustrated in FIG. 20A, in the first modified example to the second embodiment of the present invention, first, user information and device information may be registered (S10), and the server 100 may receive medical information (S3010).

As in FIG. 20A, when the device 200 performs the medical examination-by-interview, as in the third modified example of the first embodiment described previously, the device 200 may obtain user specific information (S2020), and transmit medical examination-by-interview request information of the user to the server 100 (S2031) when information about the medical examination-by-interview request is input from the user (S2030).

The server 100 may provide medical examination-by-interview information to the device terminal 230 in response to the medical examination-by-interview request information of the device terminal 230 (S1011), and the device terminal 230 may perform the medical examination-by-interview for the user according to the medical examination-by-interview inquiry information (S2032) to generate medical examination-by-interview result information.

The device terminal 230 may store the generated medical examination-by-interview result information in the memory 232 of the device 200 and transmit the information to the server 100 (S2033). In addition, the device terminal 230 may receive the medical information of the user from the server 100 (S1034) in order to use the medical information in calculating the base dose information.

Thereafter, the device terminal 230 may calculate base dose information based on the personal health information including at least one of identification information about the user, medical examination-by-interview result information, or medical information (S2040).

Here, a method in which the device terminal 230 calculates the base dose information (S2040) may be substantially the same as that of FIG. 4 except that the subject is the device 200.

Specifically, the method in which the device terminal 230 calculates the base dose information (S2040) may include the steps of calculating, by the device 200, the first base dose information (S2041), calculating, by the device 200, the second base dose information by reflecting the personal health variable value (S2042), calculating, by the device 200, the third base dose information by performing at least one of the maximum serving amount correction and the minimum serving amount correction (S2043), and performing, by the device 200, redundant correction to calculate the fourth base dose information (S2044).

The device terminal 200 may transmit the calculated base dose information to the server 100 (S2050), the server may transmit personalized result information to the user terminal 300 based on the calculated base dose information, and the user terminal may output the personalized result information (S3030).

Thereafter, at the first time point during the first period, as illustrated in FIG. 20B, the device 200 may perform the step of obtaining the device input information from the user (S2110) to obtain the user specific information, the first current condition information, and the dispensing command information.

Thereafter, the device 200 may perform the step of reflecting the change amount information at the first time (S2120) in order to calculate the first dosage form composition information at the first time by reflecting the first current condition information.

Next, the device 200 may control the dosage form to be dispensed (S2180) according to the first dosage form composition information and the dispensing command information at the first time point, generate dispensing amount information corresponding to the first time point by counting the dispensed dosage forms, and output the dispensing result information (S2190).

Thereafter, the device 200 may transmit the dispensing amount information at the first time point to the server 100 (S2191), the server 100 may reflect the dispensing amount information at the first time point and transmit taking-a-dose management information corresponding to the first current condition information at the first time point to the user terminal 300 (S1180), and the user terminal 300 may output the taking-a-dose management information (S3050).

Thereafter, at the second time point during the second period, the device 200 may perform the step of obtaining the device input information from the user (S2110) to obtain the user specific information, the second current condition information, and the dispensing command information.

Thereafter, the device 200 may perform the step of reflecting the change amount information at the second time point (S2220) in order to calculate the second dosage form composition information at the second time point by reflecting the second current condition information.

Next, the device 200 may control the dosage form to be dispensed according to the second dosage form composition information and the dispensing command information of the second time point (S2280), generate dispensing amount information corresponding to the second time point by counting the dispensed dosage form, and output dispensing result information (S2290).

Thereafter, the device 200 may transmit the dispensing amount information of the second time point to the server 100 (S2291), the server 100 may reflect the dispensing amount information at the second time point and transmit taking-a-dose management information corresponding to the second current condition information at the second time point to the user terminal 300 (S1280), and the user terminal 300 may output the taking-a-dose management information (S3050).

In the first and second embodiments so far, although the case where the user receives the dosage form from the device 200 at the first time point or the second time point, which is any one time point during the first period or the second period, has been described as an example, the user may receive the dosage form from the device 200 at a plurality of time points during the first period, and also receive the dosage form from the device 200 at a plurality of time points during the second period.

That is, in the present invention, the time point at which the device 200 provides the the dosage form to the user in the first period or the second period may be divided into a plurality of divided time points. That is, each of the first time point in the first period and the second time point in the second period may include a first divided time point and a second divided time point in each of the first or second period.

Hereinafter, as described above, a case in which the device 200 divides and provides the dosage form according to the present invention will be described in detail.

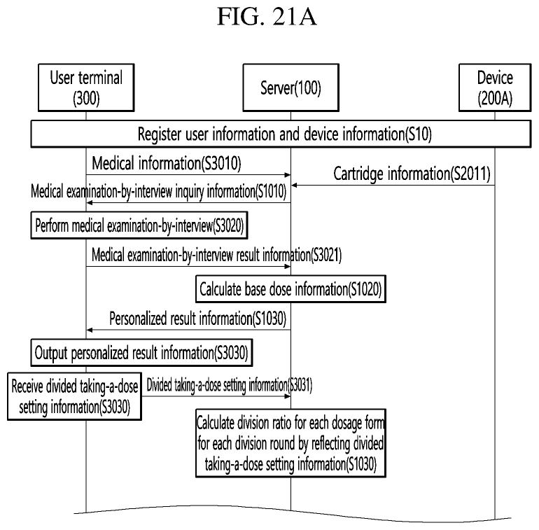

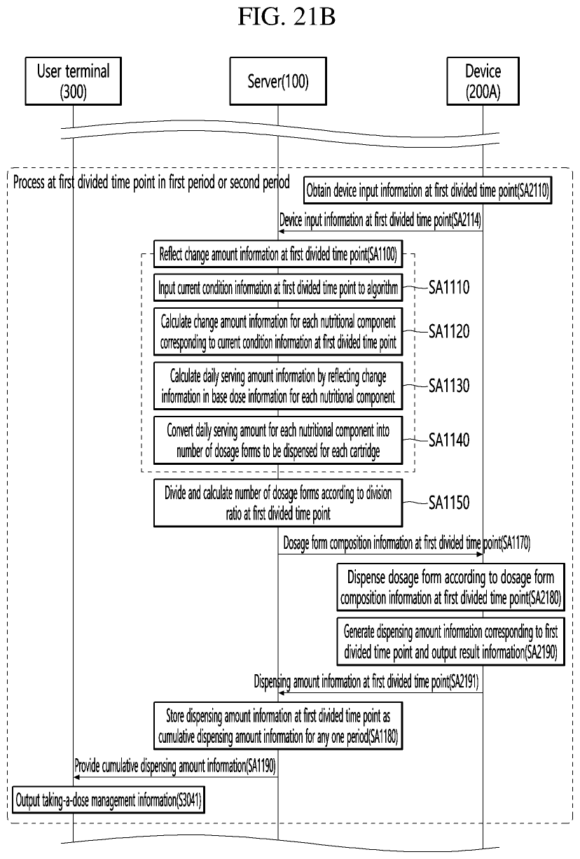

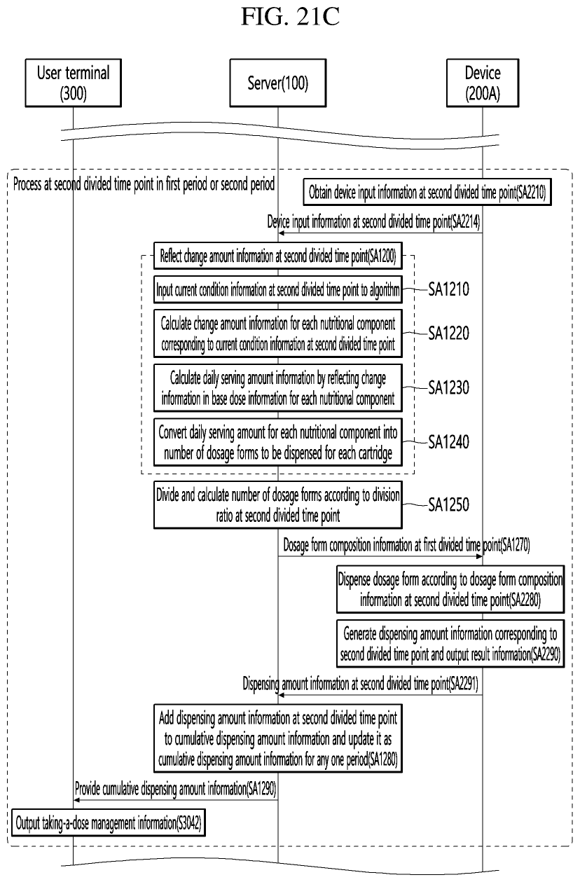

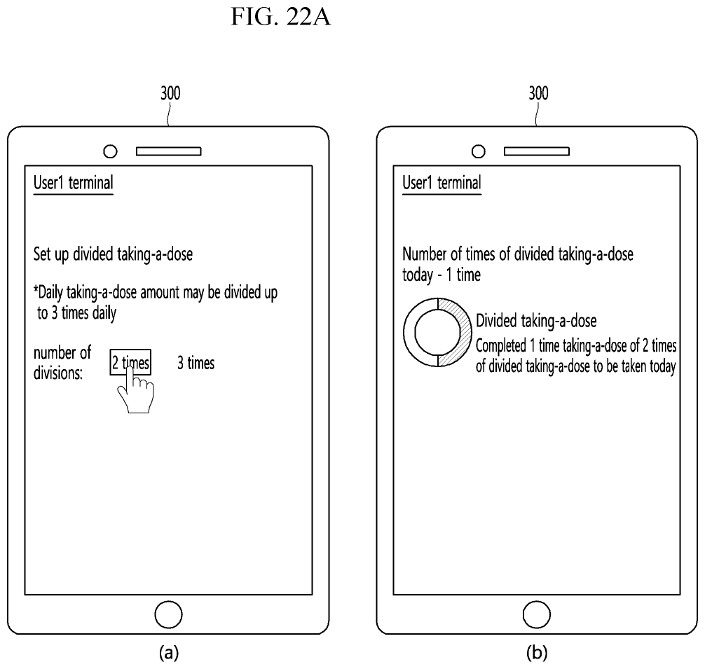

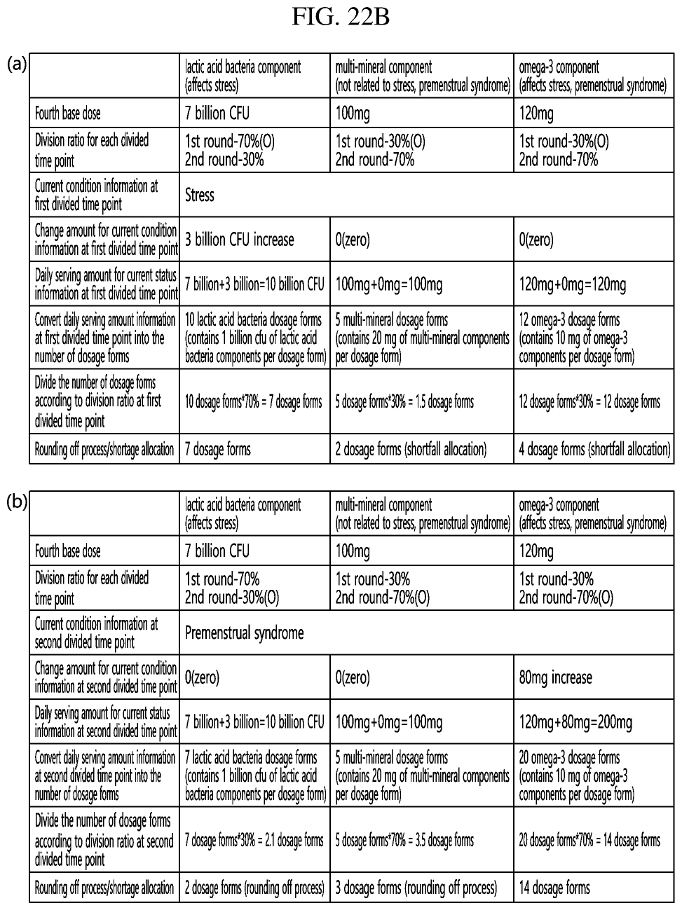

FIGS. 21A to 21C are diagrams for explaining a third embodiment of the method in which the service providing server 100, the user terminal 300, and the device 200 illustrated in FIG. 1 operate in interlocked with each other. FIG. 22A is a diagram for explaining an example of setting divided taking-a-dose through the user terminal 300 and an example of displaying the number of times of divided taking-a-dose by the user terminal 300 in FIG. 21A, and FIG. 22B is a diagram for explaining an example of the method of reflecting the change amount information at the first and second division time points in FIGS. 21B and 21C.

In the description of the third embodiment of the present invention, the contents overlapping with the contents described in the first and second embodiments described above are replaced with the contents described previously, and other parts are mainly described.

Hereinafter, the divided time point may mean a time point at which the device 200 receives current condition information is received from the user in order to provide the dosage form for each round according to divided taking-a-dose setting information during any one period of the first period and the second period.

In addition, the first and second divided time points may refer to specific time points belonging to any one period, and the second divided time point may mean a time point temporally spaced apart from the first divided time point. More specifically, the first and second divided time points may belong to the same period. For example, the first divided time point may be 9 am on October 11, and the second divided time point may be 2 pm on October 11, which is the same period as the first divided time point.

As illustrated in FIG. 21A, in the third embodiment of the present invention, first, the step of registering the user information and device information (S10) may be performed.

Next, the user terminal 300 may receive medical examination-by-interview information from the server 100 (S1010), perform a medical examination-by-interview (S3020) and generate medical examination-by-interview result information, and transmit the information to the server 100 (S3021).

In addition, the server 100 may further receive medical information in addition to the medical examination-by-interview result information from the user terminal 300 (S3010), and may receive cartridge information from the device 200 (S2011). The server 100 may ascertain the type of dosage form currently possessed by the device 200 using the cartridge information, and the cartridge information may be used later when calculating base dose information (S1020) or calculating a division ratio.

Thereafter, the server 100 may calculate base dose information for each nutritional component or each cartridge optimized for the personal health condition of the user from the personal health information including at least one of user identification information, medical examination result information, or medical information (S1020). The server 300 may transmit personalized result information to the user terminal 300 based on the calculated base dose information, and the user terminal may output the personalized result information (S3030).

The user terminal 300 may provide an interface for divided taking-a-dose setting and receive setting information for divided taking-a-dose from the user (S3030).

Here, the divided taking-a-dose means the fact that the device 200 provides the dosage form to the user at a plurality of times in any one period, rather than providing the dosage form to the user only at any one time point in any one period of the first and second periods. To this end, an application for dividing and providing the dosage form may be pre-stored and installed in the user terminal 300.

The user terminal 300 may provide, for example, an interface as illustrated in (a) of FIG. 22A in order to receive setting information for divided taking-a-dose from the user. As illustrated in (a) of FIG. 22A, the user terminal 300 may receive the number of times of divisions (e.g., two times) selected by the user (e.g., User 1) on the interface page for divided taking-a-dose setting as divided taking-a-dose setting information and transmit the divided taking-a-dose setting information to the server 100 (S3031). As another example, when the user terminal 300 may recommend the number of times of divisions to the user and obtain an input for approving the recommended number of times of divisions from the user, the user terminal 300 may transmit the divided taking-a-dose setting information according to the recommended number of times of divisions to the server 100 (S3031).

In addition, although not illustrated, the user terminal 300 may receive alarm setting information from the user for each number of times of divisions. A more detailed description of a process in which the user terminal 300 provides a taking-a-dose alarm to the user will be described later.

Next, as illustrated in FIG. 21A, the server 100 may calculate the division ratio for each dosage form for each divided round by reflecting the divided taking-a-dose setting information (S1030). The server 100 may ascertain the type of dosage form currently possessed by the device 200 by using the cartridge information in the process of calculating the division ratio. In the third embodiment of the present invention, a case in which the first division time point is the time point of the first divided round and the second division time point is the time point of the second divided round in relation divided rounds will be described as an example.

When calculating the division ratio, the server 100 may calculate the division ratio differently for each dosage form according to the characteristics of nutritional components contained in the dosage form possessed by the device 200 by considering cartridge information, characteristics of nutritional components, and the alarm setting information for each number of times of divisions together.

For example, it is ascertained that, from the cartridge information, the device 200 has a first dosage form (e.g., containing lactic acid bacteria) that is well absorbed on an empty stomach, a second dosage form (e.g., containing multi-mineral component) that causes gastrointestinal disorders on an empty stomach, and a third dosage form (e.g., containing Omega-3 component) that has a good absorption rate in the body after a meal, and the number of times of divisions may be set to two times (first round: 7:00 am, second round: 7:00 pm).

In this case, as illustrated in (a) and (b) of FIG. 22B, the server 100 may calculate a division ratio as 70% for the first round (e.g., 7 am) and as 30% for the second round (e.g., 7 p.m.) for the first dosage form (e.g., containing lactic acid bacteria component) by reflecting the fact that the absorption rate on an empty stomach of the first dosage form (e.g., containing lactic acid bacteria component) is good.

Also, as illustrated in (a) and (b) of FIG. 22B, the server 100 may calculate a division ratio as 30% for the first round (e.g., 7 am) and 70% for the second round (e.g., 7 p.m.) for the second dosage form (e.g., containing multi-mineral component) and for the third dosage form (e.g., containing Omega-3 component) by reflecting the fact that that the second dosage form causes gastrointestinal disorders on an empty stomach and that the third dosage form has a good absorption rate in the body after a meal.

Calculation of the division ratio as described above can be calculated before the first period by inputting the device information and the division ratio setting information in a division ratio setting table, and the dose (i.e., number) of the dosage form may be divided according to the division ratio at the time point at which the user receives the dosage form from the device 200.

If the user has a chronic disease and there is a taking-a-dose rule for a specific dosage form related to the chronic disease, the user may set the division ratio for the specific dosage form according to the taking-a-dose rule through the user terminal 300.

Thereafter, as illustrated in FIG. 21B, the device 200 may perform the device input: information obtainment step (SA2110) at the first division time point of any one period of the first period and the second period to obtain, for example, user specific information, current condition information at the first division time point, and dispensing command information as device input information at the first division time point, and transmit the information to the server 100 (SA2114).

When the server 100 receives the device input information at the first division time point, the server 100 divides the number of dosage forms according to the division ratio at the first division time after performing the step of reflecting the change amount information at the first division time point (SA1100) in order to calculate the dosage form composition information at the first division time point.

More specifically, in the step of reflecting the change amount information at the first divided time point (SA1100), first, the server 100 may input current condition information at the first divided time point into an algorithm (SA1110) and calculate change amount information for each nutritional component or each cartridge corresponding to the current condition information at the first divided time point based on the third base dose (SA1120).

Next, the server 100 may calculate the daily serving amount at the first divided time point by including the change amount according to the current condition information at the first divided time point in the fourth base dose for each nutritional component or each cartridge (SA1130), and convert the daily serving amount for each nutritional component or each cartridge into the number of dosage forms to be dispensed for each cartridge (SA1140). A more detailed description of each step of the method of reflecting the change amount information is the same as described above, and thus an additional description will be omitted.

Thereafter, the server 100 may divide and calculate the type and number of dosage forms for each nutritional component or each cartridge (SA1150) according to the daily serving amount at the first divided time point according to the division ratio at the first divided time point.

The server 100 may generate dosage form composition information at the first divided time point using the number of dosage forms calculated by being divided and then transmit the information to the device 200 (SA1170).

Next, the device 200 may dispense the dosage form from each cartridge 212 according to the dosage form composition information at the first divided time point (SA2180), generate dispensing amount information by counting the number of dosage forms dispensed from each cartridge 212, output dispensing result information (SA2190), and transmit the dispensing result information to the server (SA2191).

Thereafter, after receiving dispensing amount information about the type and number of dispensed dosage forms corresponding to the first divided time point from the device 200, the server 100 may store the dispensing amount information at the first divided time point as cumulative dispensing amount information for any one period (SA1180) and transmit the cumulative dispensing amount information for any one period to the user terminal 300 (SA1190).

The user terminal 300 may provide information about the number of times of divided taking-a-dose of the user, as illustrated in (b) of FIG. 22A, using the cumulative dispensing amount information of the user and output the information as taking-a-dose management information (S3041) based on dose information for each dispensed nutritional component or each cartridge accumulated until the first divided time point using the dispensing amount information for each dosage form dispensed at the first divided time point.

Thereafter, as illustrated in FIG. 21C, the device 200 may perform a device input information obtainment step (SA2210) at the second divided time point during the same period as any one period to which the first divided time point belongs, obtain user specific information, current condition information at the second divided time point, and dispensing command information as device input information at the second divided time point, and transmit the information to the server 100 (SA2214).

When the device input information at the second divided time point is received, the server 100 may divide the number of dosage forms according to the division ratio at the second divided time point after performing the step of reflecting the change amount information at the second divided time point (SA1200) in order to calculate the dosage form composition information at the second divided time point.

More specifically, in the step of reflecting the change amount information at the second divided time point (SA1200), first, the server 100 may input current condition information at the second divided time point into an algorithm (SA1210) and calculate change amount information for each nutritional component or each cartridge corresponding to the current condition information at the second divided time point (SA1220).

Next, the server 100 may calculate the daily serving amount at the second divided time point (SA1230) by including the change amount according to the current condition information at the second divided time point in the fourth base dose of each nutritional component or each cartridge, and convert the daily serving amount for each nutritional component or each cartridge into the number of dosage forms to be dispensed for each cartridge (SA1240).

Thereafter, the server 100 may divide and calculate the type and number of dosage forms for each nutritional component according to the daily serving amount at the second divided time point (SA1250) according to the division ratio at the second divided time point.

The server 100 may generate dosage form composition information at the second divided time point using the number of dosage forms calculated by being divided and then transmit the information to the device 200 (SA1270).

Next, the device 200 may dispense the dosage form from each cartridge 212 (SA2280) according to the dosage form composition information at the second divided time point, generate dispensing amount information by counting the number of dosage forms dispensed from each cartridge 212, output dispensing result information (SA2290), and transmit the dispensing result information to the server (SA2291).

Thereafter, after receiving the dispensing amount information about the type and number of dispensed dosage forms corresponding to the second divided time point from the device 200, the server 100 may add the dispensing amount information at the second divided time point to the cumulative dispensing amount information including the dispensing amount information at the first divided time point and update it as cumulative dispensing amount information for any one period (SA1280), and transmit the cumulative dispensing amount information up to the second divided time point to the user terminal 300 (SA1290). The user terminal 300 may output dose management information based on the cumulative dispensing amount information up to the second divided time point (S3042).

Here, when the server 100 divides the number of dosage forms according to the daily serving amount according to the division ratio, the server 100 may calculate the number of dosage forms divided for each round by multiplying the division ratio for each round by the number of dosage forms according to the daily serving amount.

When the number of dosage forms divided according to the division ratio occurs as a value containing a value below the decimal point rather than an integer, the value below the decimal point can be rounded off to make the value an integer.

In addition, when a shortage occurs in the number of dosage forms according to the daily serving amount due to the rounding off process according to the division ratio, the dosage form may be provided by allocating the shortage to the first round.

For example, when the number of dosage forms according to the daily serving amount is 11, and the division ratio for each round is set to 30% for the first round and 70% for the second round, the division ratio can be calculated as 3.3 for the first round and 7.7 for the second round. In this case, the server 100 may allocate the number of dosage forms for each round by dividing the number of dosage forms into 4 for the first round and 7 for the second round.

In this way, each of the dosage form composition information at the first and second divided time points provided by the server 100 may have a value obtained by assigning daily serving amount information for each dosage form to be dispensed during any one period thereto according to a division ratio.

At each of the first and second divided time points, base doses for each nutritional component or each cartridge applied in order to reflect the change amount information at each of the first and second divided time points may be the same.

For example, it is assumed that, as illustrated in (a) and (b) of FIG. 22B, the device 200 includes a lactic acid bacteria dosage form (e.g., contains lactic acid bacteria-affects stress), a multi-mineral dosage form (e.g., contains multi-mineral component-affects exercise), and an Omega-3 dosage form (e.g., contains Omega-3 component-affects premenstrual syndrome).

In this case, as illustrated in (a) and (b) of FIG. 22B, the server 100 may commonly use 7 billion CFU of the lactic acid bacteria component, 100 mg of the multi-mineral component, and 120 mg of the Omega-3 component, as the third base dose information for calculating change amount information at each of the first and second divided time points.

In addition, the daily serving amount may have a value calculated by reflecting the change amount according to the current condition information at the first divided time point or the current condition information at the second divided time point in the fourth base dose information for each nutritional component or each cartridge.

Therefore, when the current condition information at the first divided time point and the current condition information at the second divided time point are different, the daily serving amount at the first divided time point and the daily serving amount at the second divided time point may be different or the same for each nutritional component or each cartridge.

For example, it is assumed that, as illustrated in (a) and (b) of FIG. 22B, as the division ratio for each dosage form, the lactic acid bacteria dosage form is set to 70% at the first divided time point (first round) and 30% at the second divided time point (second round), and the multi-mineral dosage form and the Omega-3 dosage form are set to 30% at the first divided time point (first round) and 70% at the second divided time point (second round), respectively.

As illustrated in (a) of FIG. 22B, thereafter, for example, when stress is obtained as current condition information at the first divided time point, the server 100 may calculate 3 billion CFU increase for lactic acid bacteria component, 0 mg (zero) for Omega-3 component, and 0 mg (zero) for multi-mineral component as the change amount for each nutritional component or each cartridge.

In such a case, the server 100 may calculate 10 billion CFU of the lactic acid bacteria component, 100 mg of the multi-mineral component, and 120 mg of the Omega-3 component as the daily serving amount for each nutritional component or each cartridge at the first divided time point, and convert the daily serving amount of each nutritional component or each cartridge into the number of dosage forms, and calculate 10 lactic acid bacteria dosage forms, 5 multi-mineral dosage forms, and 12 Omega-3 dosage forms.

Here, as illustrated in (a) of FIG. 22B, the server 100 may divide the number of dosage forms for each dosage form according to the division ratio at the first divided time point, and may divide lactic acid bacteria dosage forms into 7 dosage forms (10 dosage forms*70%), multi-mineral dosage forms into 1.5 dosage forms (5 dosage forms*30%), and Omega-3 dosage forms into 3.6 dosage forms (12 dosage forms*30%), perform an integer-making process that reflects the shortage due to the rounding off process in the first round, and allocates 7 lactic acid bacteria dosage forms, 2 multi-mineral dosage forms, and 4 Omega-3 dosage forms, thereby calculating the dosage form composition information at the first divided time point.

In addition, for example, as illustrated in (b) of FIG. 22B, when the premenstrual syndrome is obtained as the current condition information at the second divided time point, the server 100 may calculate 0 CFU (zero) for the lactic acid bacteria component, 80 mg increase for Omega-3, and 0 mg (zero) for the multi-mineral component, as the change amount for each nutritional component or each cartridge.

In such a case, as illustrated in (b) of FIG. 22B, the server 100 may calculate 7 billion CFU of the lactic acid bacteria component, 100 mg of the multi-mineral component, and 200 mg of the Omega-3 component, as daily servings for each nutritional component or each cartridge at the second divided time point, convert the daily serving amount of each nutritional component or each cartridge into the number of dosage forms, calculates 2.1 lactic acid bacteria dosage forms, 3.5 multi-mineral dosage forms, and 14 Omega-3 dosage forms, and then allocate 2 lactic acid bacteria dosage forms, 3 multi-mineral dosage forms, and 14 Omega-3 dosage forms by performing the rounding off process.

In this way, when the current condition information at the first divided time point is different from the current condition information at the second divided time point, since the daily serving amount for each nutritional component or each cartridge at the first divided time point (e.g. 10 billion CFU of lactic acid bacteria, 100 mg of multi-mineral component, 120 mg of Omega-3 component) and the daily serving amount for each nutritional component or each cartridge at the second divided time point (e.g. 7 billion CFU of lactic acid bacteria, 100 mg of multi-mineral component, 200 mg of Omega-3 component) are calculated by reflecting the current condition information at the first and second divided time points, the daily serving amount may have different values for each nutritional component or each cartridge.

However, unlike this, when the current condition information at the first divided time point and the current condition information at the second divided time point are the same or the current condition information at the time of the first and second divided time points is not affected, the daily serving amount for each nutritional component or each cartridge at the first and second divided time points may be the same (e.g., multi-mineral component).

In addition, unlike the example of FIG. 22B, the daily serving amount for each nutritional component or each cartridge according to the current condition information obtained at each of the first and second divided time points for any one period may be substantially the same as the daily serving amount when all information identical to the current condition information obtained at the first and second divided time points at any one time point during any one period.

For example, when stress is obtained as current condition information at the first divided time point during the first period and premenstrual syndrome is obtained as current condition information at the second divided time point during the first period, an optional combination of the daily serving amount (e.g. 10 billion of lactic acid bacteria, 100 mg of multi-minerals, 200 mg of Omega-3) for each nutritional component or each cartridge at each divided time point may be the same as the daily serving amount for each nutritional component or each cartridge when both stress and premenstrual syndrome are obtained as current condition information during the first period.

In the third embodiment of the present invention as described above, user convenience can be further increased by allowing the user to take a plurality of dosage forms at a desired time rather than taking the plurality of dosage forms at once.

For example, if the user has a chronic disease and needs to take a dosage form related to the chronic disease a plurality of times a day, the device 200 may provide dosage forms over a plurality of times according to the prescription of dosage forms related to the chronic disease, so that the convenience of the user having the chronic disease can be further increased.

Meanwhile, in some cases, the user may not take the dosage form at the first divided time point. In this case, the server 100 may calculate dosage form composition information at the second divided time point in several ways.

For example, the server 100 may calculate dosage form composition information at the second divided time point without considering whether or not to take it at the first divided time point. In other words, the server 100 may calculate the dosage form composition information the second divided time point, as in FIG. 21B.

As another example, the server 100 may calculate, by considering not taking the dosage form at the first divided time point, the dosage form composition information at the second divided time point by reflecting the dosage form composition information at the first divided time point. As a more specific example, the server 100 may calculate the dosage form composition information at the second divided time point according to the division ratio obtained by adding the division ratio at the first divided time point and the division ratio at the second divided time point. As another more specific example, the server 100 may finally calculate the dosage form composition information at the second divided time point by adding the dosage form composition information at the first divided time point to the dosage form composition information at the second divided time point calculated without considering whether or not to take at the first divided time point. Accordingly, the user can take the dosage form, which is not taken at the first divided time point, at the second divided time point.

Whether or not to consider as to whether or not the dosage form is taken at the previous divided time point may be selected by the user. For example, when the user takes the dosage form at the second divided time point, the server 100 may receive an input from the user in real time whether or not to consider not taking the dosage form at the first divided time point. As another example, the server 100 may receive an input from the user in advance whether or not to consider as whether the dosage form is to be taken at the previous divided time point and sets it, and calculate dosage form composition information at the second divided time point, with or without considering as to whether or not the dosage form is to be taken at the first divided time point according to the set value.

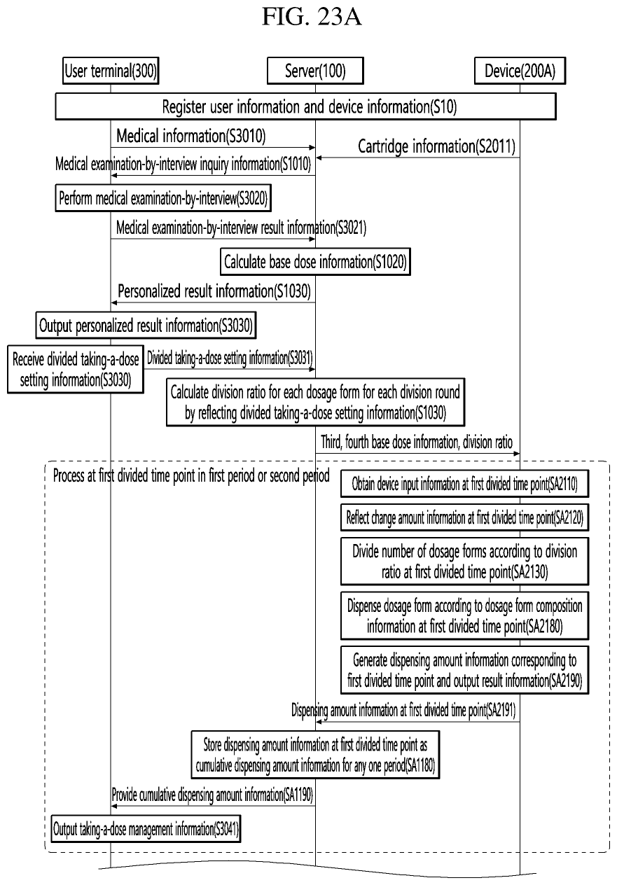

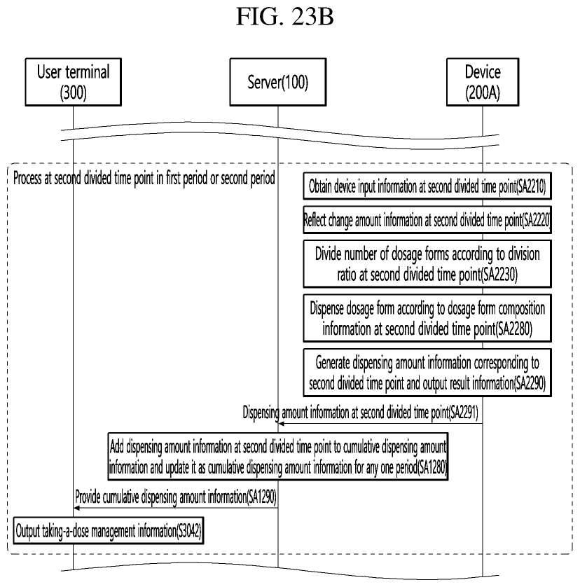

In the matters as described above, it has been described that the server 100 calculates the dosage form composition information at the second divided time point when the dosage form is not taken at the first divided time point, but the dosage form composition information may be calculated in the device 200 as illustrated in FIG. 23 to be described later.

FIGS. 23A and 23B are diagrams for explaining a first modified example of the third embodiment described in FIGS. 21A to 21C.

In the first modified example of the third embodiment of the present invention, the description of overlapping contents with the previous first to third embodiments is replaced with the contents described above, and the other contents are mainly explained.

As illustrated in FIGS. 23A and 23B, in the first modified example of the third embodiment is different from the third embodiment in that the device terminal 230, not the server 100, reflects the change amount information at the first and second divided time points and the number of dosage forms is divided according to the division ratio at the first divided time point, and the remaining parts may be substantially the same as the third embodiment.

As illustrated in FIG. 23A, in the first modified example of the third embodiment, the step of registering user information and device information (S10) may be performed.

Next, the user terminal 300 receives the medical examination-by-interview inquiry information from the server 100 (S1010) and performs a medical examination-by-interview (S3020) to generate medical examination-by-interview result information, and transmit the generated medical examination-by-interview result information to the server 100 (S3021).

The server 100 may further receive medical information from the user terminal 300 in addition to the medical examination-by-interview result information (S3010), and may receive cartridge information from the device 200 (S2011).

Thereafter, the server 100 may calculate base dose information for each nutritional component or each cartridge optimized for personal health condition of the user (S1020) from m personal health information including at least one of user identification information, medical examination-by-interview result information, or medical information, and transmit personalized result information to the user terminal 300 based on the base dose information, and the user terminal 300 may output the personalized result information.

The user terminal 300 may receive setting information for divided taking-a-dose from the user (S3030) and transmit the information to the server 100 (S3031), and the server 100 may calculate the division ratio for each dosage form for each divided round by reflecting the divided taking-a-dose setting information (S1030).

The server 100 may transmit the base dose information and information about the division ratio of the user registered in the device 200 to the device 200. Here, the base dose information transmitted to the device 200 may include any one of first to third base dose information and fourth base dose information. In the first modified example of the third embodiment, a case in which the third base dose information and the fourth base dose information are included in the base dose information transmitted to the device 200 will be described as an example.

The device 200 may obtain device input information from the user at a first divided time point during any one period of the first period and the second period (SA2110). Here, the device input information at the first divided time point may include, for example, user specific information, current condition information at the first divided time point, and dispensing command information.

Thereafter, in the step of reflecting the change information at the first divided time point (SA2120), the device 200 may calculate change amount information for each nutritional component or each cartridge based on the third base dose using the current condition information at the first divided time point, calculate the daily serving amount for each nutritional component or each c cartridge at the first divided time point by including the change amount according to the current condition information at the first divided time point in the fourth base dose of each nutritional component or each cartridge, and convert the daily serving amount into the number of dosage forms to be dispensed for each cartridge.

Next, in the step of dividing the number of dosage forms according to the division ratio at the first divided time point (SA2130), the device 200 may calculate dosage form composition information at the first divided time point by dividing the number of dosage forms according to the daily serving amount at the first divided time point according to the division ratio at the first divided time point.

Thereafter, the device 200 may dispense the dosage form from each cartridge 212 according to the dosage form composition information at the first divided time point (SA2180), generate dispensing amount information by counting the number of dosage forms dispensed from each cartridge 212, output dispensing result information (S2190), and transmit the dispensing result information to the server (SA2191).

The server 100 may receive dispensing amount information about the type and number of dispensed dosage forms corresponding to the first divided time point from the device 200, and then store the dispensing amount information at the first divided time point as cumulative dispensing amount information for any one period (SA1180), and transmit the cumulative dispensing amount information for any one period to the user terminal 300 (SA1190), and the user terminal may output dose management information based on the cumulative dispensing amount information (S3041).

Thereafter, as illustrated in FIG. 23B, the device 200 may obtain device input information from the user at a second divided time point (SA2210) during any one period to which the first divided time point belongs. Here, the device input information at the second divided time point may include, for example, user information, specific current condition information at the first divided time point, and dispensing command information.

Thereafter, in the step of reflecting the change information at the second divided time point (SA2220), the device 200 may calculate change amount information for each nutritional component or each cartridge based on the third base dose using the current condition information at the second divided time point, calculate the daily serving amount for each nutritional component or each cartridge at the second divided time point by including the change amount according to the current condition information at the second divided time point in the fourth base dose of each nutritional component or each cartridge, and convert the daily serving amount into the number of dosage forms to be dispensed for each cartridge.

Next, in the step of dividing the number of dosage forms according to the division ratio at the second divided time point (SA2230), the device 200 may calculate dosage form composition information at the second divided time point by dividing the number of dosage forms according to the daily serving amount at the second divided time point according to the division ratio at the second divided time point.

Thereafter, the device 200 may dispense the dosage form from each cartridge 212 according to the dosage form composition information at the second divided time point (SA2280), generate dispensing amount information by counting the number of dosage forms dispensed from each cartridge 212, output dispensing result information (S2290), and transmit the dispensing result information to the server (SA2291).

The server 100 may receive dispensing amount information about the type and number of dispensed dosage forms corresponding to the second divided time point from the device 200, and then add the dispensing amount information at the second divided time point to cumulative dispensing amount information including the dispensing amount information at the first divided time point and update the information as cumulative dispensing amount information for any one period (SA1280), and transmit the cumulative dispensing amount information up to the second divided time point to the user terminal 300 (SA1290), and the user terminal may output taking-a-dose management information based on the cumulative dispensing amount information (S3042).

Here, when the current condition information at the first and second divided time points is different, the daily serving amount at the first divided time point calculated by the device 200 and the daily serving amount at the second divided time point may be different from each other.

In addition, when the current condition information at the first and second divided time points is equal to each other, the daily serving amount calculated by the device 200 at the first divided time point and the daily serving amount at the second divided time point may be equal to each other.

In the third embodiment and the modified example of the third embodiment according to the present invention so far, the case where one device 200 divides the dosage form according to the division ratio within any one period and provides it at the first divided time point and the second divided time point has been described as an example.

However, the present invention is not limited thereto, and it is also possible for the plurality of devices 200 to provide dosage forms according to the division ratio at each divided time point. An explanation of this is as follows.

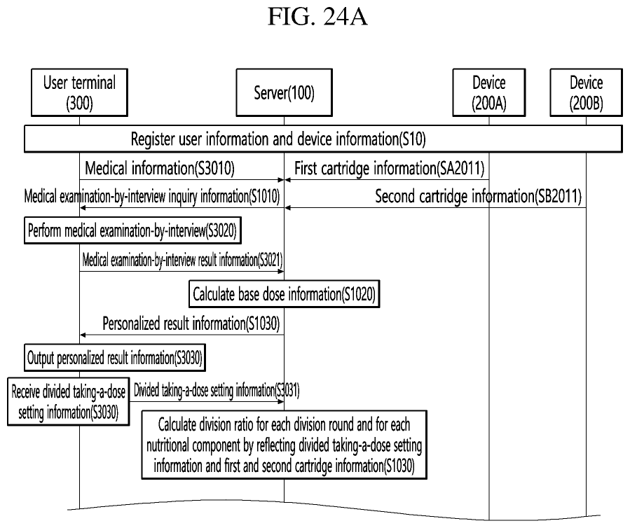

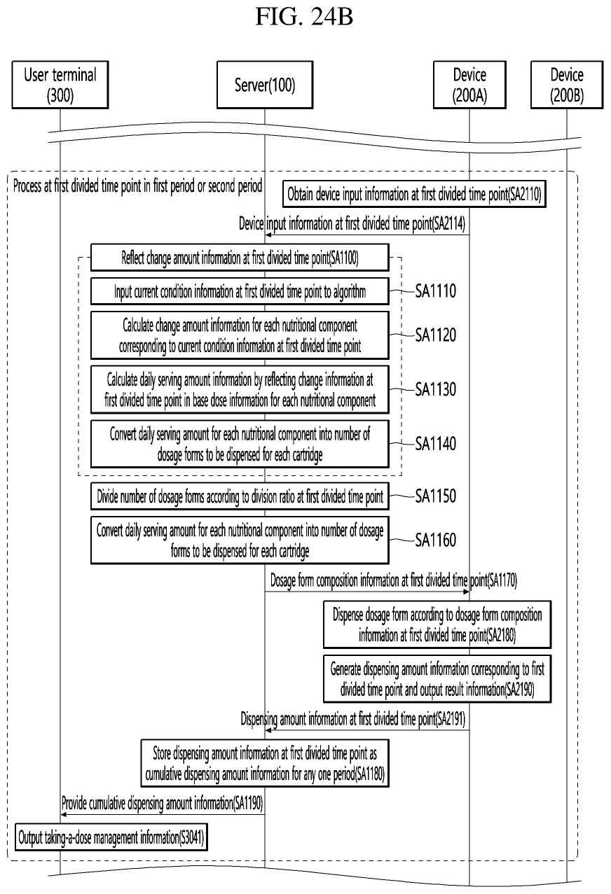

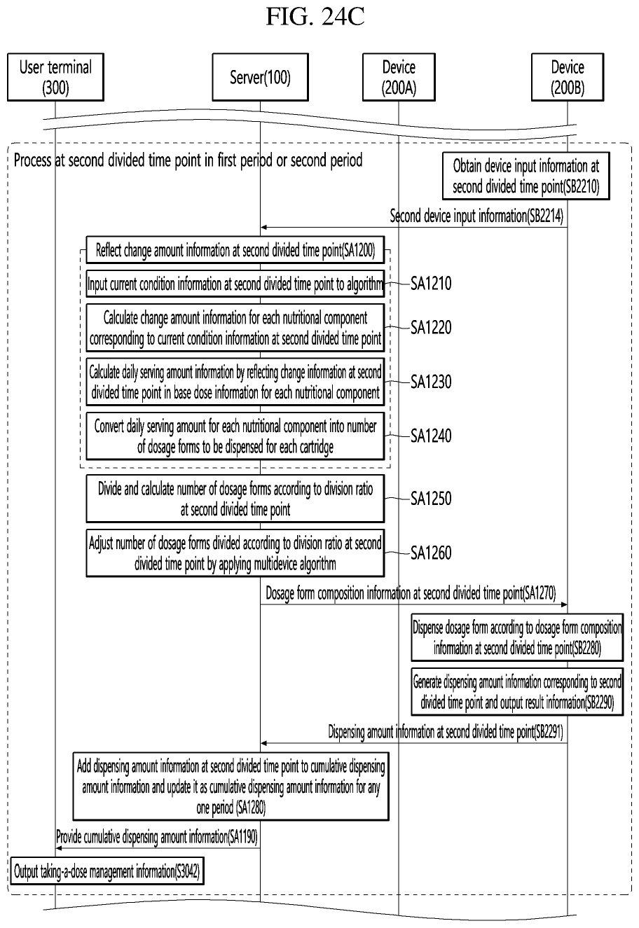

FIGS. 24A to 24C are diagrams for explaining a fourth embodiment of a method in which the service providing server 100, the user terminal 300, and the device 200 illustrated in FIG. 1 operate in interlocked with each other.

In the fourth embodiment of the present invention, descriptions of the contents overlapping with those described in the first to third embodiments are replaced with the contents described above, and other parts are mainly described.

As illustrated in the fourth embodiment, the device 200 may include a first device 200A and a second device 200B. Here, the first device 200A and the second device 200B may be provided at locations spaced apart from each other. For example, when the first device 200A is provided in a house of the user, the second device 200B may be provided in a company where the user works.

In the present invention, as in the fourth embodiment, when a plurality of devices 200 register the same user, all of the plurality of devices 200 may provide a dosage form to the user. Specifically, the plurality of devices 200 may provide the dosage form to the user in the divided provision manner described in the third embodiment or the first modified example of the third embodiment described above.

In the fourth embodiment of the present invention, as illustrated in FIG. 24A, first, the step of registering user information and device information (S10) may be performed.

In the step of registering user information and device information (S10), the server 100 may receive user identification information and identification information of each of the first and second devices 200A and 200B from the user terminal 300, receive identification information of each of the first and second devices 200A and 200B from each of the first and second devices 200A and 200B, set a registered user account and the first and second devices 200A and 200B to be interlocked with each other.

Next, the user terminal 300 may receive medical examination-by-interview inquiry information from the server 100 (S1010) and perform a medical examination-by-interview (S3020) to generate medical examination-by-interview result information, and transmit the generated medical examination-by-interview result information to the server 100 (S3021).

The server 100 may further receive medical information from the user terminal 300 (S3010) in addition to the medical examination-by-interview result information, receive first cartridge information from the first device 200A (SA2011) and receive second cartridge information from the second device 200B (SB2011). Accordingly, the server 100 may ascertain dosage forms for each nutritional component or each cartridge possessed by the first device 200A and dosage forms for each nutritional component or each cartridge possessed by the second device 200B.

Thereafter, the server 100 may calculate base dose information for each nutritional component or each cartridge optimized for personal health condition of the user (S1020) from the personal health information including at least one of user identification information, medical examination-by-interview result information, and medical information, and the first and second cartridge information. The server 300 may transmit personalized result information to the user terminal 300 based on the calculated base dose information, and the user terminal may output personalized result information (S3030).

The user terminal 300 may receive setting information for divided taking-a-dose from the user (S3030) and transmit the information to the server 100 (S3031), and the server 100 may calculate a division ratio for each dosage form possessed by the first and second devices 200A and 200B for each divided round by reflecting the divided taking-a-dose setting information and the first and second cartridge information. The division ratio may be calculated differently depending on the characteristics of the nutritional components contained in the dosage form. In relation to the divided round, a case in which the first divided time point is a time point of the first divided round is the second divided time point is a time point of the second divided round will be described as an example.

Next, as illustrated in FIG. 24B, the first device 200A may obtain input information of the first device 200A (SA2110) at a first divided time point during any one period of the first period and the second period. For example, the first device 200A may obtain user specific information, current condition information at the first divided time point, and dispensing command information from the user as input information of the first device 200A, and transmit the input information of the first device 200A to the server 100 (SA2114).

The server 100 may perform a step of reflecting the change amount information at the first divided time point (SA1100) using the input information of the first device 200A obtained at the first divided time point.

Specifically, the server 100 may input the current condition information at the first divided time point received from the first device 200A into an algorithm (SA1110) in order to reflect the information as change amount information at the first divided time point, calculate change information for each nutritional component or each cartridge corresponding to the current condition information at the first divided time point based on the third base dose (SA1120), calculate the daily serving amount for each nutritional component or each cartridge at the first divided time point by including the change amount according to the current condition information at the first divided time point in the fourth base dose of each nutritional component or each cartridge (SA1130), and convert the daily serving amount into the number of dosage forms to be dispensed for each cartridge (SA1140).

Next, the server 100 may divide the number of dosage forms according to the daily serving amount at the first divided time point according to the division ratio at the first divided time point (SA1150).

Thereafter, the server 100 may calculate dosage form composition information at the first divided time point (SA1160) by adjusting the number of divided dosage forms according to the division ratio at the first divided time point by applying a multi-device algorithm, and transmit the dosage form composition information at the first divided time point to the first device 200A (SA1170).

Specifically, as a result of the server 100 referring to the first and second cartridge information, when the first dosage form is identified as being provided in only one device 200 among the plurality of devices 200, the multi-device algorithm may adjust the number of divided first dosage forms according to the division ratio so that all of the daily serving amount for the first dosage form is dispensed from only one device 200.

For example, as a result of the server 100 referring to the first and second cartridge information, there may be a case where the first device 200A is identified as having a lactic acid bacteria dosage form, a multi-mineral dosage form, and a vitamin B dosage form, and the second device 200A is identified as having the multi-mineral dosage form, the vitamin B dosage form, and an Omega-3 dosage form.

In such a case, the multi-device algorithm may adjust the number of lactic acid bacteria dosage forms calculated according to the division ratio so that all of the daily serving amount of the lactic acid bacteria dosage form is dispensed at the first divided time point for the lactic acid bacteria dosage form that only the first device 200A has.

In addition, the multi-device algorithm may adjust the number of lactic acid bacteria dosage forms calculated according to the division ratio to 0 (zero) so that the Omega-3 dosage form is not dispensed at the first divided time point for the Omega-3 dosage form that only the second device 200B has.

In addition, the multi-device algorithm may allow the number of multi-mineral dosage forms and vitamin B dosage forms, that the first and second devices 200A and 200B commonly have, to be maintained according to the division ratio at the first divided time point.

Thereafter, the first device 200A may dispense the dosage form from each cartridge 212 of the first device 200A (SA2180) according to the dosage form composition information at the first divided time point, generate dispensing amount information by counting the number of dosage forms dispensed from each cartridge 212, output dispensing result information (SA2190), and then transmit the information to the server (SA2191).

After receiving the dispensing amount information about the type and number of dispensed dosage forms corresponding to the first divided time point from the first device 200A, the server 100 may store the dispensing amount information at the first divided time point as cumulative dispensing amount information for any one period (SA1180), and transmit the cumulative dispensing amount information for any one period to the user terminal 300 (SA1190). The user terminal 300 may output taking-a-dose management information based on the cumulative dispensing amount information up to the first divided time point (S3041).

Next, as illustrated in FIG. 24C, the second device 200B may obtain input information of the second device 200B (SA2210) at a second divided time point during any one period. For example, the second device 200B may obtain user specific information, current condition information at the second divided time point, and dispensing command information from the user as input information of the second device 200B, and the transmit input information of the second device 200B to the server 100.

The server 100 may perform a step of reflecting the change amount information at the second divided time point point (SA1200) using the input information of the second device 200B obtained at the second divided time point.

Specifically, the server 100 may input the current condition information at the second divided time point received from the second device 200B into an algorithm (SA1210) in order to reflect the information as change amount information at the second divided time point, calculate change amount information for each nutritional component or each cartridge corresponding to the current condition information at the second divided time point based on the third base dose (SA1220), calculate the daily serving amount for each nutrient component or cartridge at the second divided time point by including the change amount according to the current condition information at the second divided time point in the fourth base dose of each nutritional component or each cartridge (SA1230), and convert the daily serving amount into the number of dosage forms to be dispensed for each cartridge (SA1240).

Next, the server 100 may divide the number of dosage forms according to the daily serving amount at the second divided time point according to the division ratio at the second divided time point (SA1250).

The server 100 may calculate dosage form composition information at the second divided time point by adjusting the number of divided dosage forms according to the division ratio at the second divided time point (SA1260) by applying a multi-device algorithm, and transmit the dosage form composition information at the first divided time point to the first device 200A (SA1270).

For example, when the cumulative dispensing amount at the previous round (e.g., first divided time point) for the first dosage form is equal to the number of daily serving amounts at the first divided time point, the multi-device algorithm may adjust the number of divided first dosage forms according to the division ratio to 0 (zero) so that the first dosage form is not dispensed at the current round (e.g., the second divided time point).

In addition, when the cumulative dispensing amount up to the previous round (e.g., the first divided time point) for the second dosage form is 0 (zero), the multi-device algorithm may adjust the number of divided dosage forms according to the division ratio so that all of the daily serving amount for the second dosage form is dispensed at the current round (e.g., the second divided time point).

For example, for the lactic acid bacteria dosage form, when all of the daily serving amount was dispensed at the previous round (e.g., the first divided time point) and thus the cumulative serving amount up to the previous round became the same as the daily serving amount of the lactic acid bacteria dosage form, the number of divided lactic acid bacteria dosage forms according to the division ratio may be adjusted to 0 (zero) so that the lactic acid bacteria dosage form is not dispensed at the current round (e.g., the second divided time point).

In addition, for the Omega-3 dosage form, if the cumulative dispensing amount up to the previous round (e.g., the first divided time point) is 0 (zero), the number of divided dosage forms may be adjusted according to the division ratio so that all of the daily serving amount for Omega-3 is dispensed at the current round (e.g., the second divided time point).

In addition, when the first and second devices 200A and 200B have the multi-mineral dosage form and the vitamin B dosage form in common, the multi-device algorithm may maintain the number of dosage forms according to the division ratio at each of the first and second divided time points.

In addition, if, due to insufficient remaining amount or failure of the cartridge 212, etc., the dispensing amount information of the second dosage form (e.g., multi-mineral dosage form) at the previous round (e.g., the first divided time point) is insufficient compared to the number of second dosage forms (e.g., multi-mineral dosage form) according to the dosage form composition information at the first divided time point, the server 100 may make up for the shortage amount that could not be dispensed in the previous round (e.g., the first divided time point) in the current round (e.g., the second divided time point).

Specifically, the server 100 may adjust the number of second dosage forms to be dispensed at the current round (e.g., the second divided time point) by including the number of the dosage forms that amounts to the shortage amount not dispensed in the previous round (e.g., the first divided time point) in the number of second dosage forms (e.g., multi-mineral dosage form) in the current round (e.g., the second divided time point) calculated according to the division ratio.

For example, for the multi-mineral dosage form, when the number of dosage forms to be dispensed was 10 dosage forms, but the actual dispensed amount was 8 dosage forms according to the dosage form composition information at the previous round (e.g., the first divided time point), the server 100 may add two dosage forms to the number of multi-mineral dosage forms (e.g., multi-mineral dosage forms) at the current round (e.g., the second divided time point) calculated according to the division ratio and calculate the dosage form composition information at the second divided time point.

Thereafter, the second device 200B may dispense the dosage form from each cartridge 212 of the second device 200B (SB2280) according to the dosage form composition information at the second divided time point, generate dispensing amount information by counting the number of dosage forms dispensed from each cartridge 212, and output dispensing result information (SB2290), and then transmit the information to the server (SB2291).

After receiving the dispensing amount information about the type and number of dispensed dosage forms corresponding to the second divided time point from the second device 200B, the server 100 may add the dispensing amount information at the second divided time point to the cumulative dispensing amount information including the dispensing amount information at the first divided time point and update the information as the cumulative dispensing amount information for any one period (SA1280), and transmit cumulative dispensing amount information up to the second divided time point to the user terminal 300 (SA1290). The user terminal 300 may output taking-a-dose management information based on the cumulative dispensing amount information up to the second divided time point (S3042).

In the fourth embodiment of the present invention, the user can receive the dosage form through the plurality of devices 200, and thus user's convenience can be further increased.

The base dose information described above may be updated in some cases. Here, the updated base dose information may be at least one of the first to fourth base dose information described above.

For example, when the server 100 calculates and stores the first base dose information, but does not store the second to fourth base dose information, the first base dose information may be updated, when the server 100 calculates and stores up to the second base dose information, the first and second base dose information may be updated, when the server 100 calculates and stores up to the third base dose information, the first to third base dose information may be updated, and when the server 100 calculates and stores up to the fourth base dose information, the first to fourth base dose information may be updated.

For example, when personal health information of the user is updated, such as when the user performs a medical examination-by-interview or the medical information of the user is updated, the server 100 may update the base dose information based on the updated personal health information.

As another example, when specific current condition information is received more than a predetermined number of times during a predetermined period, the server 100 may update the base dose information.

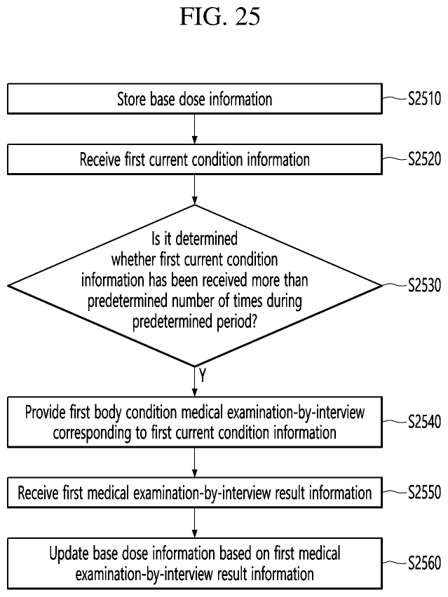

FIG. 25 is a diagram for explaining an example of a method in which the server 100 updates base dose information.

In step S2510, the server 100 may store the base dose information.

In step S2520, the server 100 may receive first current condition information.

In step S2530, the server 100 may determine whether the received first current condition information has been received more than a predetermined number of times during a predetermined period. For example, in a case where a user takes a dosage form through a device during 1 day (or 24 hours), if the predetermined period is 1 week (i.e., 7 days) and the predetermined number of times is 5 times (or 5 days), the server 100 may determine whether the first current condition information has been received five or more times during one week.

If it is determined that the first current condition information has not been received more than a predetermined number of times during a predetermined period, the server 100 may not update the base dose information. Thereafter, if second current condition information is received, the server 100 may calculate dosage form composition information using the base volume information that has not been updated and the second current condition information. The server 100 may transmit the calculated dosage form composition information to the device 200, and the device 200 may dispense the dosage form according to the received dosage form composition information.

If it is determined that the first current condition information is received more than the predetermined number of times during the predetermined period, the server 100 may update the base dose information and calculate dosage form composition information using the updated base dose information and the first current condition information.

If it is determined that the first current condition information is received more than the predetermined number of times during the predetermined period, in step S2540, the server 100 may provide a first body condition medical examination-by-interview corresponding to the first current condition information. As an example, the server 100 may provide the first body condition medical examination-by-interview through the user terminal 300.

In step S2550, the server 100 may receive first medical examination-by-interview result information, which is a result of the of the user performing the first body condition medical examination-by-interview, through the user terminal. For example, when the user performs the first body condition medical examination-by-interview provided through the user terminal 300, the user terminal 300 may transmit the first medical examination-by-interview result information, which is the result of the user performing the first body condition medical examination-by-interview, to the server 100, and accordingly, the server 100 may receive the first the first medical examination-by-interview result information from to the user terminal 300.

In step S2560, the server 100 may update the base dose information based on the received first medical examination-by-interview result information. In some cases, the base dose information for each nutritional component or each cartridge may be changed according to the first current condition information. For example, base dose information of nutritional components or cartridges that affect the first current condition information may be updated, and base dose information of nutritional components or cartridges that do not affect the first current condition information may not be updated.

Here, the content of calculating the base dose information based on the medical examination-by-interview result information described above may be similarly applied to updating the base dose information based on the first first medical examination-by-interview result information, and thus duplicate descriptions are omitted. Thereafter, when the second current condition information is received, the server 100 may calculate dosage form composition information using the updated base volume information and the second current condition information. The server 100 may transmit the calculated dosage form composition information to the device 200, and the device 200 may dispense the dosage form according to the received dosage form composition information.

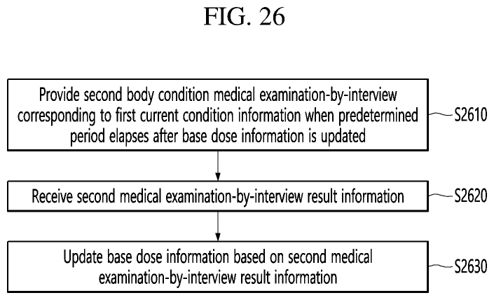

The updated base dose information may be updated again if certain conditions are satisfied. For example, when a predetermined period elapses after the base dose information is updated, the base dose information may be updated again. FIG. 26 is a diagram for explaining an example of a method in which the server 100 updates the base dose information again.

In step S2610, the server 100 may provide a second body condition medical examination-by-interview corresponding to the first current condition information when the predetermined period elapses after the base dose information is updated. As an example, the server 100 may provide the second body condition medical examination-by-interview through the user terminal 300. Here, the second body condition medical examination-by-interview may be the same as the first body condition medical examination-by-interview. Alternatively, the second body condition medical examination-by-interview may be different from the first body condition medical examination-by-interview.

Alternatively, in step S2610, if the first current condition information is received the first number of times or more or the second number of times, which is less than the first number of times, or less during a predetermined update period after the base dose information is updated, the server 100 may provide the second body condition medical examination-by-interview corresponding to the first current condition information again.

In step S2620, the server 100 may receive second medical examination-by-interview result information, which is the result of the user performing the second body condition medical examination-by-interview. For example, when the user performs the second body condition medical examination-by-interview provided through the user terminal 300, the user terminal 300 may transmit the second medical examination-by-interview result information, which is the result of the user performing the second body condition medical examination-by-interview, to the server 100, and accordingly, the server 100 may receive the second the first medical examination-by-interview result information from to the user terminal 300.

In step S2630, the server 100 may update the base dose information based on the received second medical examination-by-interview result information. For example, if it is determined that the first current condition information about the user is resolved according to the second medical examination-by-interview result information, the server 100 may return the base dose information to the base dose information before updating based on the first medical examination-by-interview result information. As another example, if it is determined that the first current condition information is not resolved for the user according to the second medical examination-by-interview result information, the server 100 may maintain the base dose information updated based on the first medical examination-by-interview result information. As another example, the server 100 may calculate base dose information based on the second medical examination-by-interview result information, just as base dose information is calculated based on the medical examination result information described above, a redundant description thereof will be omitted. In some cases, the nutritional component or cartridge for which the base dose information is updated may vary according to the first current condition information. For example, base dose information of nutritional components or cartridges that affect the first current condition information may be updated, and base dose information of nutritional components or cartridges that do not affect the first current condition information may not be updated.

Thereafter, when the second current condition information is received, the server 100 may calculate dosage form composition information using the updated base volume information and the second current condition information. The server 100 may transmit the calculated dosage form composition information to the device 200, and the device 200 may dispense the dosage form according to the received dosage form composition information.

In addition, after analyzing the first or second medical examination-by-interview result information to update the base dose information, the server 100 may provide body condition medical examination-by-interview result information obtained by analyzing the first or second medical examination-by-interview result information, to the user terminal 300, and the user terminal 300 may output the body condition medical examination-by-interview result information.

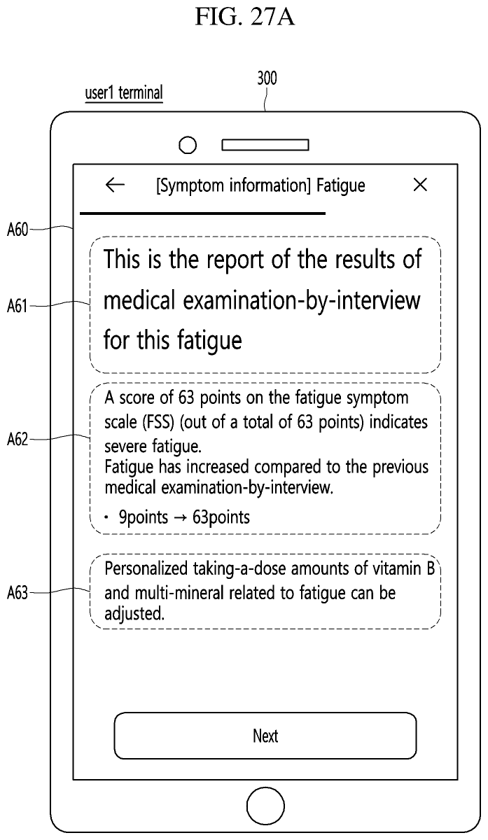

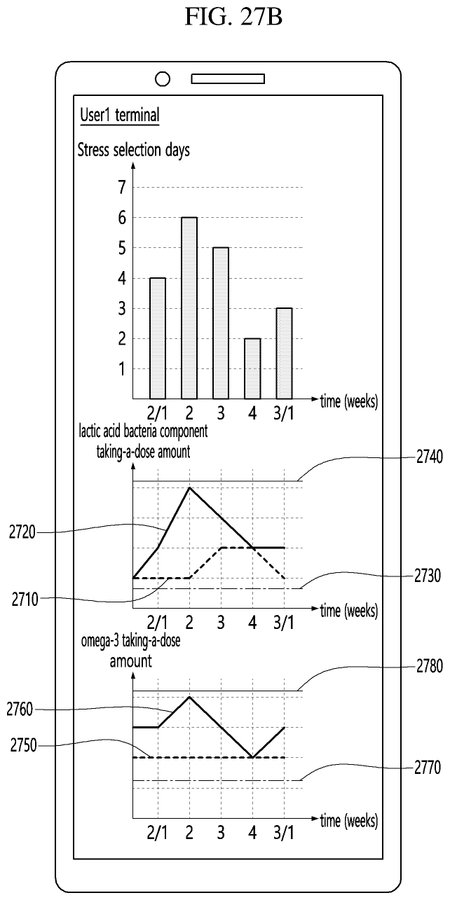

FIG. 27A is a diagram for explaining an example in which a user terminal outputs the body condition medical examination-by-interview result information in FIG. 25 or 26, and FIG. 27B is a diagram for explaining an example in which the user terminal 300 displays cumulative taking-a-dose amount information, base dose information, average serving amount, minimum serving amount, and maximum serving amount.

As illustrated in FIG. 27A, the body condition medical examination-by-interview result information A60 output by the user terminal 300 may include information about an end or category of body condition medical examination-by-interview related to current condition information A61 (e.g., fatigue), a degree of change in symptoms related to the current condition information A62 (e.g., 9 points=>63 points) and description thereof, a type of nutritional component A63 (e.g., vitamin B, multi-mineral) for which the base dose is updated, etc.

In addition, although not illustrated in FIG. 27A, in the body condition medical examination-by-interview result information A63, an immediately-before base dose and immediately-after base dose of the nutritional component whose base dose is changed may be displayed together.

As illustrated in FIG. 27B, the server 100 may provide, through the user terminal 300, the number of selected days for each period for specific current condition information (e.g., stress).

As illustrated in FIG. 25, if the predetermined period for updating the base dose information is 1 week and the predetermined number of times is 5 times, in FIG. 27B, since the stress was selected on the 6th in the 2nd week of February, the condition for updating the base dose information was satisfied. Accordingly, the server 100 may provide a first body condition medical examination-by-interview corresponding to stress, receive first medical examination-by-interview result information, which is a result of performing the first body condition medical examination-by-interview, and update base dose information based on the first medical examination-by-interview result information.

In this case, assuming that the lactic acid bacteria component affects stress and the Omega-3 component does not affect stress, the base dose information of the lactic acid bacteria component is updated and changed based on the first medical examination-by-interview result information, and the base dose information of the Omega-3 component may be maintained without being updated. Accordingly, in FIG. 27B, in the third week of February, base dose information 2710 of the lactic acid bacteria component increases, and base dose information 2750 of the Omega-3 component is maintained.

As illustrated in FIG. 26, if the predetermined period for updating the base dose information again is 2 weeks, in FIG. 27B, the time point after the 4th week of February corresponds to this. Accordingly, the server 100 may provide a second body condition medical examination-by-interview corresponding to stress, receive second medical examination-by-interview result information, is which a result of performing the second body condition medical examination-by-interview, and update base dose information based on the second medical examination-by-interview w result information. Accordingly, In FIG. 27B, according to the second medical examination-by-interview result information, the base dose information 2710 of the lactic acid bacteria component in the first week of March may return to before the increase in the third week of February or decrease.

In addition, as illustrated in FIG. 27B, the server 100 may provide the average serving amounts 2720 and 2760, the minimum serving amounts 2730 and 2770, and the maximum serving amounts 2740 and 2780 for each nutritional component or each cartridge through the user terminal 300. Here, the average serving amounts 2720 and 2760 mean the average of the serving amounts provided to the user by reflecting the current condition information in the base dose information 2710 and 2750.

In FIGS. 25 to 27B, the case where the user takes the dosage form once a day through the device has been described as an example. However, if the user sets divided taking-a-dose, for example, three times a day, and receives the dosage form through the device, the server may receive current condition information from the device three times during one day.

In this case, when the same current condition information (e.g., stress) is received three times during one day, even if the server receives the same current condition information (e.g., stress) three times on the same day, the server can count it once. However, if stress is received as the current condition information in the first round, fatigue as the current condition information in the second round, and exercise as the current condition information in the third round, the server can count stress, fatigue, and exercise once each.

The user terminal 300 may output an alarm encouraging the user to take a dose (hereinafter, referred to as ‘taking-a-dose encouragement alarm’). For example, the user terminal 300 may visually display the taking-a-dose encouragement alarm. As another example, the user terminal 300 may aurally output the taking-a-dose encouragement alarm.

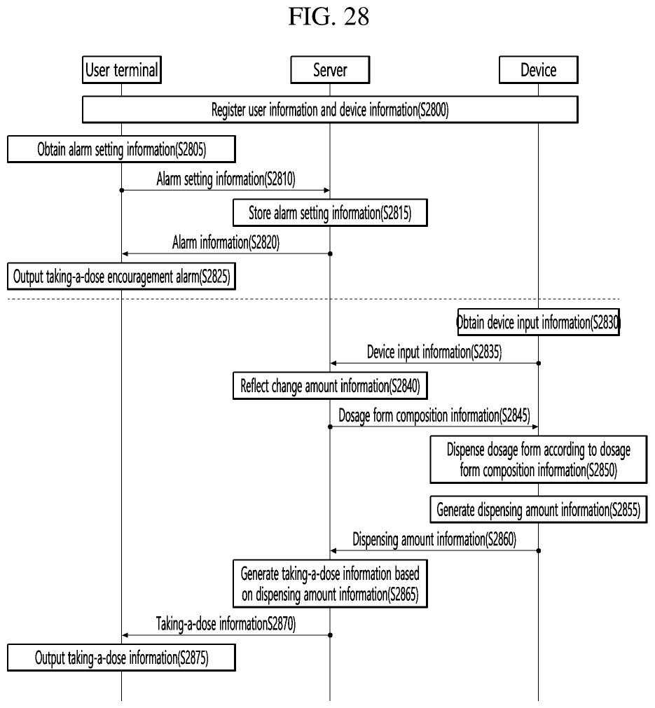

FIG. 28 is a diagram for explaining an example of a method in which the user terminal 300 outputs the taking-a-dose encouragement alarm.

In FIG. 28, the description of the contents overlapping with the contents described above is replaced with the previous description, and the other parts are mainly described.

As illustrated in FIG. 28, in the method of outputting a taking-a-dose encouragement alarm according to an embodiment, first, a step of registering user information and device information (S2800) may be performed.

Next, the user terminal 300 may obtain alarm setting information from the user (S2805).

As an example, the user terminal 300 may receive taking-a-dose alarm time information from the user. Thereafter, the user terminal 300 may obtain alarm setting information by generating the alarm setting information according to the received the taking-a-dose alarm time information.

As another example, the user terminal 300 may provide recommended alarm time information. Thereafter, the user terminal 300 may obtain the alarm setting information by obtaining a user input for approving the recommended alarm time information and generating alarm setting information according to the recommended alarm time information. Here, the recommended alarm time information may be calculated by reflecting a past taking-a-dose history of the user. For example, the recommended alarm time information may be a time point at which the user took a dosage form in the past.

In the case of divided taking-a-dose, the alarm setting information may include information about a plurality of alarm times. For example, in the case of divided taking-a-dose, the alarm setting information may include information about a first alarm time for a first divided time point and information about a second alarm time for a second divided time point.

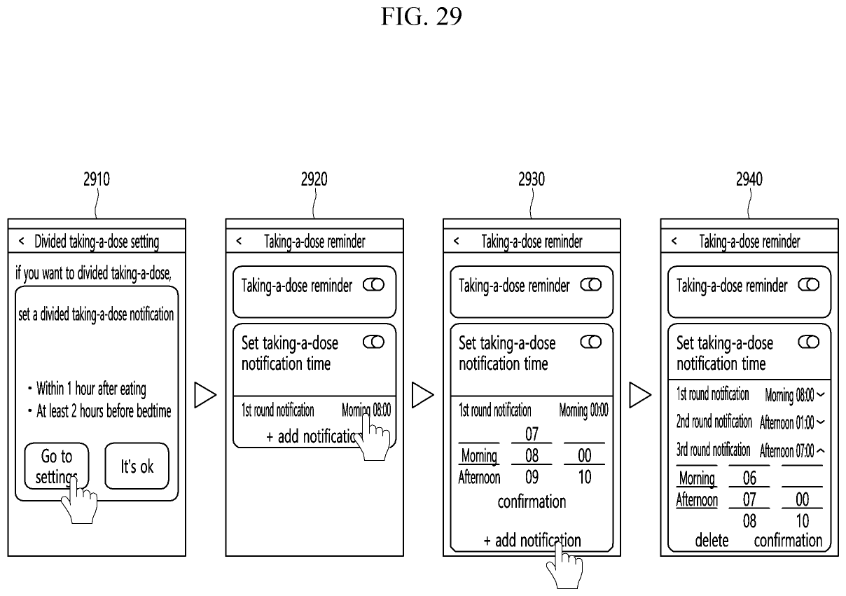

FIG. 29 is a diagram for explaining an example in which the user terminal 300 obtains alarm setting information. Although FIG. 29 describes the case of divided taking-a-dose, the user terminal 300 can similarly obtain alarm setting information from the user even when divided taking-a-dose is not set.

As illustrated in FIG. 29, the user terminal 300 may display an interface 2910 notifying setting of the divided taking-a-dose. When a user input to start setting the divided taking-a-dose alarm is obtained, the user terminal 300 may display an interface 2920 for setting an alarm time for each divided round. When a user input for selecting a specific divided round is obtained, the user terminal 300 may display an interface 2930 for setting an alarm time for the selected divided round. The user may set an alarm time on the interface 2930. When a user input for adding an alarm for the selected divided round is obtained, the user terminal 300 may display an interface 2940 for setting an alarm time for the next divided round. If this process is repeated for all divided rounds, the user terminal 300 may obtain alarm setting information including alarm times for all divided rounds.

The user terminal 300 may transmit the obtained alarm setting information to the server 100.

The server 100 may store the received alarm setting information (S2815). Thereafter, the server 100 may transmit alarm information according to the alarm setting information to the user terminal 300 (S2820). For example, the server 100 may transmit alarm information to the user terminal 300 when the taking-a-dose alarm time comes according to the alarm setting information.

The user terminal 300 may output the taking-a-dose encouragement alarm according to the received alarm information (S2825). In some cases, the user terminal 300 may output the taking-a-dose encouragement alarm only when the user does not take the dosage form. To this end, the user terminal 300 may receive the taking-a-dose information of the user from the server 100 and determine whether or not the user takes the dosage form based on the received taking-a-dose information, and output the taking-a-dose encouragement alarm only when it is determined that the user has not taken the dosage form. Alternatively, the server 100 may determine whether the user takes the dosage form based on the taking-a-dose information and transmit alarm information to the user terminal 300 only when it is determined that the dosage form is not taken, and the user terminal 300 may output the taking-a-dose encouragement alarm according to the received alarm information, and output the taking-a-dose encouragement alarm only when the user does not take the dosage form.

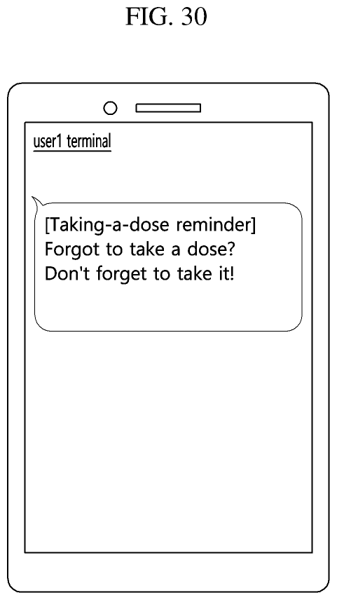

FIG. 30 is a diagram for explaining an example in which the user terminal 300 visually displays the taking-a-dose encouragement alarm. As illustrated in FIG. 30, the user terminal 300 may display the taking-a-dose encouragement alarm on the display. As another example, it is also possible to output the taking-a-dose encouragement alarm information as a sound.

Thereafter that, as illustrated in FIG. 28, the device 200 may obtain device input information (S2830) and transmit the obtained device input information to the server 100 (S2835). When the server 100 calculates dosage form composition information by reflecting change amount information according to the received device input information (S2840) and transmits the calculated dosage form composition information to the device 200 (S2845), the device 200 may dispense a dosage form according to the received dosage form composition information (S2850), generate dispensing amount information, and output the dispensing result information (S2855). Thereafter, the device 200 transmits the dispensing amount information to the server 100 (S2860). When the server 100 generate taking-a-dose information based on the received dispensing amount information (S2865) and transmits the generated taking-a-dose information to the user terminal 300 (S2870), the user terminal 300 may output the received taking-a-dose information (S2875). Here, the taking-a-dose information refers to information indicating whether or not the user has taken a dose. For example, the taking-a-dose information may include information indicating a taking-a-dose state and information indicating a non-taking-a-dose.

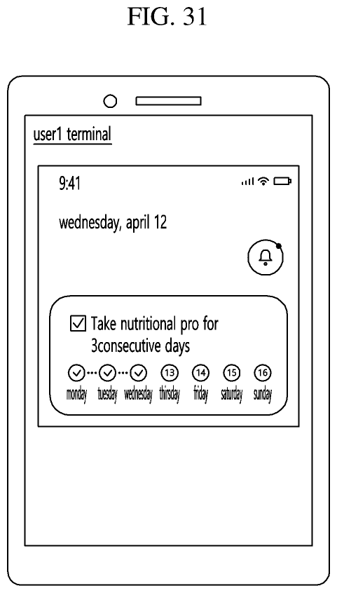

FIG. 31 is a diagram for explaining an example in which the user terminal 300 displays taking-a-dose information.

As illustrated in FIG. 31, the user terminal 300 may display taking-a-dose information on the display. In FIG. 31, the case where the time point at which the taking-a-dose information is displayed is Wednesday and the dosage form is taken from Monday to Wednesday is illustrated.

Meanwhile, in FIG. 28, the server 100 is described as reflecting the change amount information, but reflecting the change information amount can be performed in various ways as described above. For example, as illustrated in FIG. 19, the device 200 may reflect change amount information.

In some cases, the user terminal 300 may obtain alarm modification information from the user. Preset alarm setting information may be modified according to the alarm modification information.

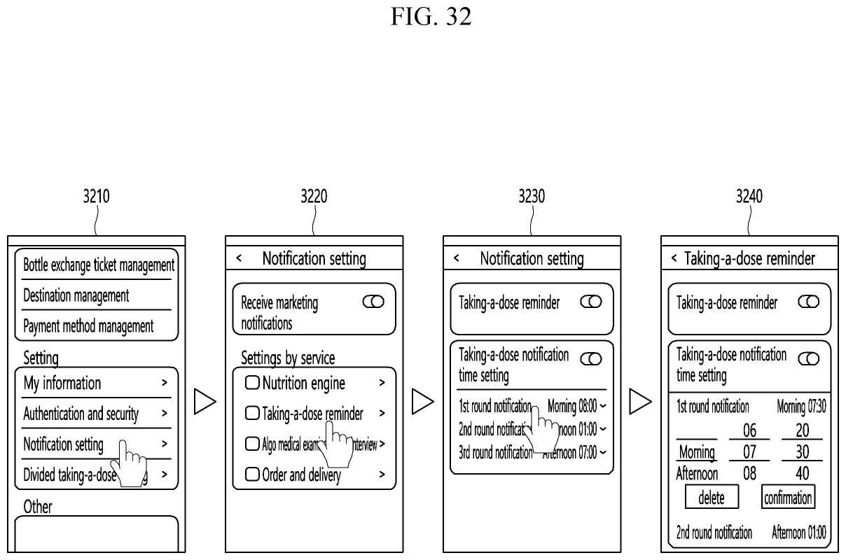

FIG. 32 is a diagram for explaining an example in which the user terminal 300 obtains the alarm modification information. Although FIG. 32 describes the case of divided taking-a-dose, the user terminal 300 may similarly obtain the alarm modification information from the user even when divided taking-a-dose is not set.

As illustrated in FIG. 32, the user terminal 300 may display an interface 3210 for checking and changing various settings including the alarm setting. When a user input for selecting alarm setting is obtained, the user terminal 300 may display an interface 3220 for checking and changing various alarm settings. Here, various alarm settings may include setting the taking-a-dose encouragement alarm. When a user input for selecting the taking-a-dose encouragement alarm is obtained, the user terminal 300 may display an interface 3230 indicating an alarm time for each divided round. When a user input for for selecting a specific divided round is obtained, the user terminal 300 may display an interface 3240 for modifying an alarm time of the selected divided round. A user can modify the alarm time on an interface 3240.

As such, in one embodiment according to the present invention, at the time point at which the combination of dosage forms is provided to the user, a combination of dosage forms optimized for the health condition of the user for each time point may be provided by reflecting current condition information on the health condition of the user.

So far, the case where the user is provided with the dosage form every day through the device 200 has been described as an example, but in some cases, there may be a period in which the user is not provided with the dosage form through the device 200 for reasons such as vacation. In preparation for such a case, the present invention provides the dosage form to the user in advance through the device 200, so that the user's convenience can be further improved.

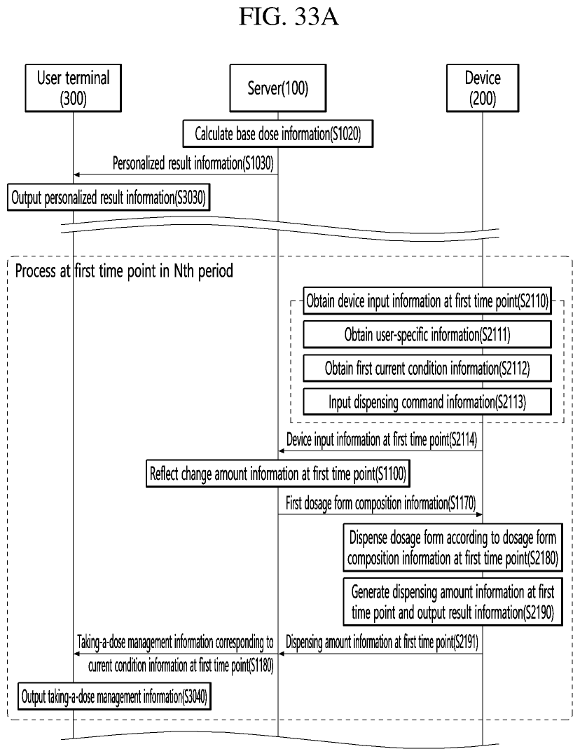

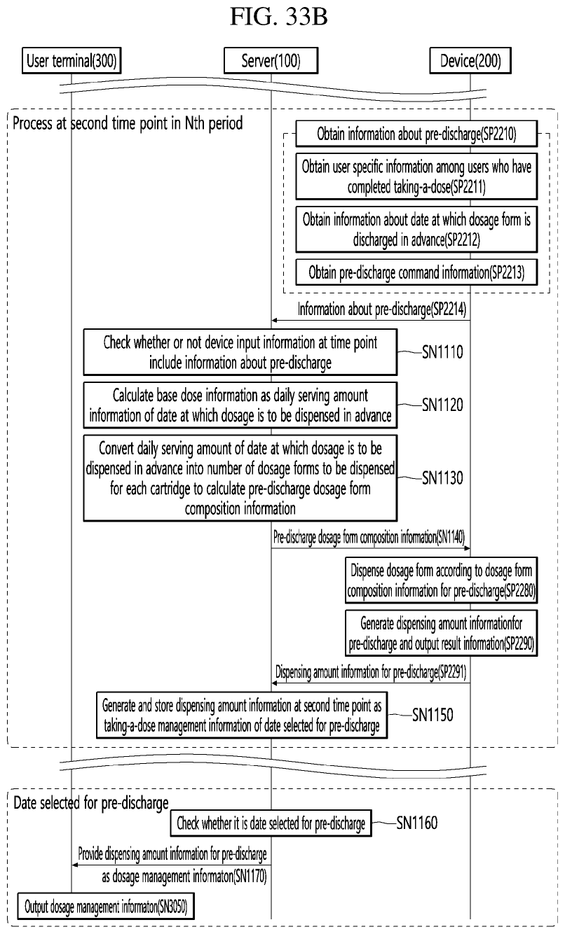

This will be described in more detail with reference to FIGS. 33A to 36. In FIGS. 33A to 36, the same content as those described above is replaced with the contents described above, and other parts are mainly described.

FIGS. 33A to 33C are diagrams for explaining an example of a method in which the server 100, the user terminal 300, and the device 200 shown in FIG. 1 operate together for pre-dispensing.

FIGS. 33A to 33C illustrate a case in which a user cannot use the device 200 while continuously receiving a dosage form through the device 200 after the base dose information is calculated.

As illustrated in FIG. 33A, the server 100, after calculating base dose information, may transmit personalized result information to the user terminal 300 based on the base dose information, and the user terminal 300 may output the personalized result information.

Thereafter, until the Nth period, the user may be provided with the dosage form through the device 200 every day. Therefore, in the Nth period, the device 200 may obtain the device 200 input information by receiving the user specific information, the first current condition information, and the dispensing command information from the user at the first time point (S2110), and then transmit the device 200 input information at the first time point to the server 100.

The server 100 may calculate the first dosage form composition information by reflecting the device 200 input information at the first time point in the base dose information according to the step of reflecting the change amount information at the first time point, and transmit the information to the device 200.

The device 200 may dispense the dosage form according to the first dosage form composition information, counting the dispensed dosage form when the dosage form is dispensed, generate dispensing amount information by counting the dispensed dosage form when the dosage form is dispensed, and output dispensing result information. In addition, the dispensing amount information at the first time point may be transmitted to the server 100.

The server 100 may generate taking-a-dose management information based on the current condition information and dispensing amount information at the first time point and transmit the taking-a-dose management information to the user terminal 300. The user terminal 300 may output the taking-a-dose management information at the first time point.

Here, the dose of each nutritional component or each cartridge dispensed up to the first time point of the Nth period may have the same value as the daily serving amount of the Nth period. That is, the dose dispensed up to the first time point of the Nth period may be a case where all of the dose to be taken by the user in the Nth period is dispensed. Accordingly, when divided taking-a-dose is set, the dispensing amount dispensed up to the first time point of the Nth period may be equal to the sum of all doses to be dispensed as the divided taking-a-dose in the Nth period.

Then, after the first time point of the Nth period, when the user intends to receive the dosage form in advance during the Nth period, the user may enter a pre-dispensing mode through the device 200.

To this end, the device 200 may obtain information about pre-dispensing from the user (SP2210). For example, the step of obtaining the information about the pre-dispensing (SP2210) may be performed by the device 200 obtaining user specific information among users who have completed taking-a-dose (SP2211), obtaining information about a date at which the dosage form is dispensed in advance (SP2212), and obtaining pre-dispensing command information (SP2213).

In addition, although not illustrated, the step of obtaining the information about the pre-dispensing (SP2210) may further include providing a user interface (UI) for entering the pre-dispensing mode.

However, since user-specific information is obtained from among users who have already completed taking-a-dose in step SP2211 of FIG. 33B, this may be determined as entering the pre-dispensing mode, and thus the step of providing a separate UI for entering the pre-dispensing mode may be omitted.

Thereafter, the 200 device may transmit the information about pre-dispensing to the server 100 (SP2214).

The server 100 may check (SN1110) whether or not the device 200 input information at the second time point during the Nth period includes information about pre-dispensing. For example, the server 100 may determine whether or not the dispensing at the second time point is the pre-dispensing by checking whether a user who has completed taking-a-dose is selected, whether there is date information, whether there is pre-dispensing command information, etc. in the device 200 input information at the second time point during the Nth period.

If it is determined that it is the pre-dispensing, the server 100 may calculate the base dose information as daily serving amount information of the date at which the dosage is to be dispensed in advance (SN1120), convert the daily serving amount of the date at which the dosage is to be dispensed in advance into the number of dosage forms to be dispensed for each cartridge, calculate pre-dispensing dosage form composition information (SN1130), and store the pre-dispensing dosage form composition information.

Thereafter, the server 100 may transmit the pre-dispensing dosage form composition information to the device 200 (SN1140), and the device 200 may dispense the dosage form from each cartridge (SP2280) according to the pre-dispensing dosage form composition information at the second time point during the Nth period, generate pre-dispensing dispensing amount information, and output pre-dispensing result information (SP2290).

Next, the device 200 may transmit the pre-dispensing dispensing amount information generated at the second time point to the server 100 (SP2291), and the server 100 may generate and store the information as taking-a-dose management information of a date selected for the pre-dispensing (SN1150) based on the pre-dispensing dispensing amount information at the second time point, and may not generate the information as the taking-a-dose management information for the Nth period. In addition, the server 100 may not provide the taking-a-dose management information of the date selected for the pre-dispensing to the user terminal 300 during the Nth period.

Thereafter, when the date selected for pre-dispensing arrives, the server 100 may check whether it is the date selected for the pre-dispensing (SN1160), and provide the taking-a-dose management information for the pre-dispensing at the second time point in the Nth period to the user terminal 300 (SN1170). The user terminal 300 may output the taking-a-dose management information for the pre-dispensing (SN3050).

In FIG. 33B, the case where the server 100 transmits the taking-a-dose management information for the pre-dispensing to the user terminal 300 on the date selected for the pre-dispensing has been described as an example, but the present invention is not necessarily limited thereto. It is also possible to transmit the information to the user terminal 300 in the Nth period. In such a case, the current taking-a-dose status information among the taking-a-dose management information may be displayed on the user terminal 300 in advance before the date selected for the pre-dispensing arrives.

Hereinafter, a method in which the device 200 operates for pre-dispensing will be described in more detail.

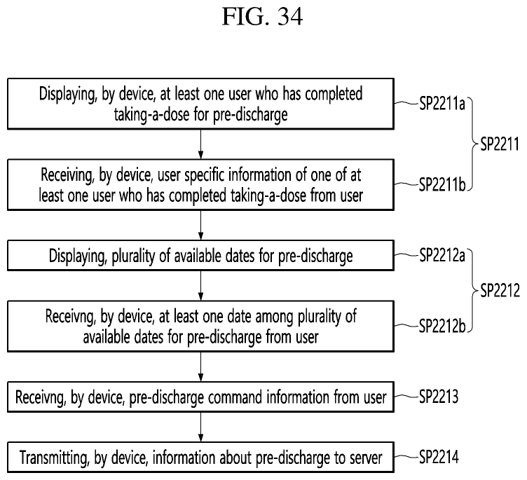

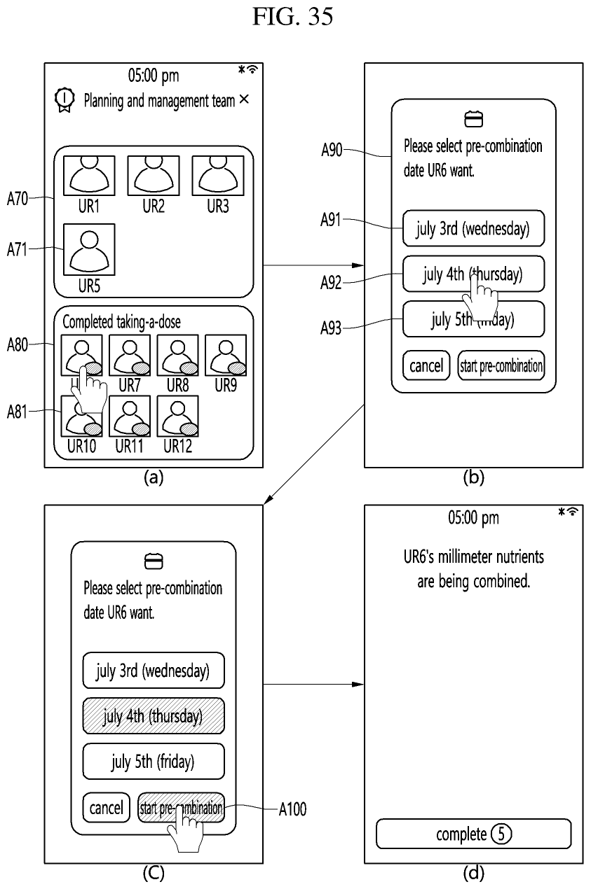

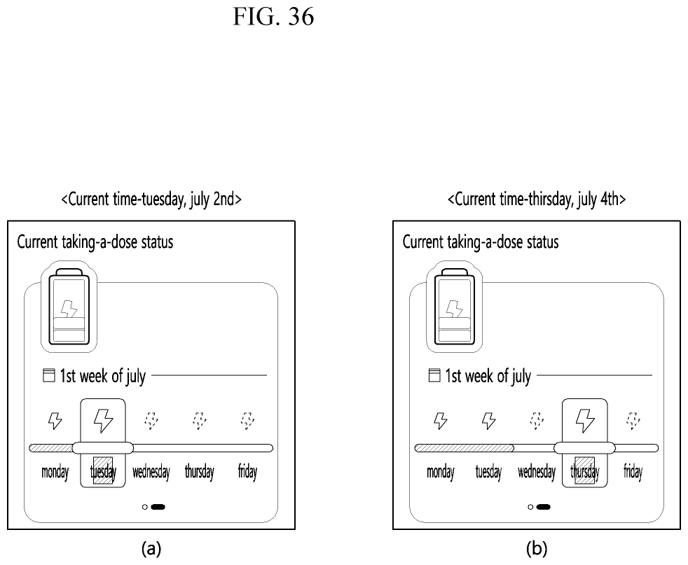

FIG. 34 is a diagram for explaining in detail the method in which the device 200 operates for pre-dispensing in FIGS. 33A to 33C, FIG. 35 is a diagram for explaining an example in which a user interface for pre-dispensing of the device 200 is output when the device 200 operates for pre-dispensing according to FIG. 34, and FIG. 36 is a diagram for explaining an example in which taking-a-dose management information is output to the user terminal 300 in relation to pre-dispensing.

The method in which the device 200 operates for pre-dispensing may include, as illustrated in FIG. 34, the steps of displaying a user (SP2211a), receiving user specific information (SP2211b), displaying an available date (SP2212a), receiving pre-dispensing date (SP2212b), receiving pre-dispensing command information (SP2213) and transmitting the information (SP2214).

In the step of displaying the user (SP2211a) of FIG. 34, the device 200 may display at least one user who has completed taking-a-dose for pre-dispensing through the terminal 230 of the device 200. For example, as illustrated in (a) of FIG. 35, the device 200 may display, based on the second time point of the Nth period, a list of users who have not taken the dose A70 who have not been provided with the dosage form through the device 200 and a list of users who have completed taking-a-dose A80 who have been provided with the dosage form through the device 200.

If a user 6 UR6 is provided with a dosage form through the device 200 at the first time point of the Nth period, the user 6 UR6 may be included in and displayed in the list of users who have completed taking-a-dose A80.

In the step of receiving the user specific information (SP2211b) of FIG. 34, the device 200 may receive user specific information of one of at least one user who has completed taking-a-dose from the user. For example, as illustrated in (a) of FIG. 35, the device 200 may receive the user 6 (UR6), which is selected by the user's selection. In this way, when the user 6 (UR6) is selected, the device 200 may obtain the user 6 (UR6) as user specific information.

In this way, when the device 200 receives the user specific information of one of the users who have completed taking-a-dose, the device 200 may display an available date for pre-dispensing as illustrated in FIG. 35 (b) while entering the pre-dispensing mode.

In the step of displaying the available date (SP2212a) of FIG. 34, the device 200 may display a plurality of available dates for pre-dispensing that arrive after the Nth period. As an example, if the Nth period is July 2nd, as illustrated in (b) of FIG. 35, three days from July 3rd A91 to July 5th A93 may be displayed as available dates for pre-dispensing. However, this is an example, the present invention is not limited thereto, and there is no problem as long as a plurality of dates are displayed after the Nth period.

Next, in the step of receiving the date for pre-dispensing (SP2212b) of FIG. 34, the device 200 may receive, from the user, input or selection of at least one date among a plurality of available dates for pre-dispensing. As an example, as illustrated in (b) of FIG. 35, the device 200 may receive, from the user, input or selection of July 4th (Thursday) A92 among the period from July 3rd to July 5th, and accordingly, the device 200 may obtain information about the date (July 4th) at which the dosage form is to be dispensed in advance. Thereafter, in the step of receiving the pre-dispensing command information (SP2213) of FIG. 34, the device 200 may receive the pre-dispensing command information from the user. For example, when the device 200 receives selection of the pre-dispensing command information A100 from the user as illustrated in (c) of FIG. 35, a screen for ending the pre-dispensing mode may be displayed as illustrated in (d) of FIG. 35.

Thereafter, in the step of transmitting the information (SP2214), the device 200 may transmit information about the pre-dispensing to the server 100.

FIG. 36 is a diagram for explaining an example in which taking-a-dose management information is output to the user terminal 300 in the Nth period and at a pre-dispensing date in relation to pre-dispensing.

As an example, if the Nth period in FIG. 33A is Tuesday, July 2nd, the user terminal 300, after receiving the taking-a-dose management information from the server 100, may output the current taking-a-dose status information, which is one of the taking-a-dose management information at the first time point, as illustrated in (a) of FIG. 36 to display that the user has received the dosage form on Tuesday, July 2nd.

However, as illustrated in FIG. 33B, when the dosage form is provided in advance for the date (e.g., Thursday, July 4th) selected by the user through the device 200 at the second time point of the Nth period, the server 100 may store the dispensing amount information at the second time point as dosage form provision information on a date selected for pre-dispensing, and may not provide the information to the user terminal 300.

Accordingly, the user terminal 300 may output the same current taking-a-dose status information as illustrated in (a) of FIG. 36 at the second time point of the Nth period.

However, the present invention is not necessarily limited thereto. It is also possible to display that the dosage form for Thursday, July 4th has been provided in advance by outputting the current taking-a-dose status information as illustrated in (b) of FIG. 36 at the second time point of the Nth period (e.g., Tuesday, July 2nd). However, a case in which the user terminal 300 outputs the current taking-a-dose status information as illustrated in (a) of FIG. 36 at the second time point of the Nth period will be described as an example.

Thereafter, when the date selected for pre-dispensing (e.g., Thursday, July 4th) arrives, the user terminal 300 may display that the user will receive a dosage form for Thursday, July 4th by outputting the current taking-a-dose status information as illustrated in (b) of FIG. 36.

In this way, when the device 200 provides the dosage form to the user in advance by pre-dispensing, the device 200 may restrict the user from receiving the dosage form again through the device 200 on a date selected for pre-dispensing.

That is, if the user is provided with the dosage form for the date (e.g., Thursday, July 4th) selected for pre-dispensing in advance, the device 200 may restrict the use of the device 200 by the user on the selected date to thereby prevent overdose.

The disclosed technical features disclosed in each embodiment and modified example of the present invention are not limited to the corresponding embodiment, and may be incorporated or modified and applied to other embodiments or modified examples unless they are incompatible with each other.

Therefore, in each embodiment, respective technical features are mainly described, but unless respective technical features are incompatible with each other, they can be merged and applied.

The present invention is not limited to the embodiments and accompanying drawings described above, and various modifications and alterations may be made thereto from the viewpoint of those skilled in the art in the field to which the present invention belongs. Therefore, the scope of the present invention should be defined not only by the claims of this specification but also by those equivalent to these claims.

1. A method for calculating dosage form composition information by an electronic device, the method comprising the steps of:
obtaining first current condition information for a user at a first time point in a first period;
calculating first dosage form composition information by reflecting the first current condition information in base dose information of the user for a plurality of nutritional components;
obtaining second current condition information for the user at a second time point in a second period; and
calculating second dosage form composition information by reflecting the second current condition information in the base dose information, wherein
the base dose information is determined before the first time point of the first period, and has a dose value personalized for the user for the plurality of nutritional components, and
in the step of calculating the second dosage form composition information, the second dosage form composition information is calculated by reflecting the second current condition information to the dose value of the base dose information used when calculating the first dosage form composition information.

2. The method according to claim 1, wherein
the base dose information is determined based on at least personal health information, and
the personal health information includes at least one of identification information, medical examination-by-interview result information, or medical information for the user.

3. The method according to claim 1, further comprising:
a step of obtaining dose information for each nutritional component before the first time point to calculate the base dose information.

4. The method according to claim 1, wherein
the first or second current condition information includes information about at least one of a mental health condition or symptom of the user, a physical health condition or symptom of the user, a mood or behavior of the user, and biometric information of the user.

5. The method according to claim 1, wherein
the first or second current condition information is collected from at least one of a device for dispensing dosage forms for the plurality of nutritional components, a user terminal of the user, or an external device for collecting the biometric information of the user.

6. The method according to claim 1, further comprising the steps of:
after the step of obtaining the first current condition information, calculating first change amount information about a first nutritional component related to the first current condition information among the plurality of nutritional components; and
after the step of obtaining the second current condition information, calculating second change information about a second nutritional component related to the second current condition information the among plurality of nutritional components.

7. The method according to claim 6, wherein
in the step of calculating the first dosage form composition information,
for the first nutritional component, a daily serving amount in the first period is calculated by including the first change amount information in base dose information about the first nutritional component, and
for a nutritional component unrelated to the first current condition information among the plurality of nutritional components, base dose information about the nutritional component unrelated to the first current condition information is calculated as the daily serving amount in the first period, and
in the step of calculating the second dosage form composition information,
for the second nutritional component, a daily serving amount in the second period is calculated by including the second change amount information in base dose information about the second nutritional component, and
for a nutritional component unrelated to the second current condition information among the plurality of nutritional components, base dose information about the nutritional component unrelated to the second current condition information is calculated as the daily serving amount in the second period.

8. An electronic device that calculates dosage form composition information, the electronic device comprising:
a memory; and
a processor, wherein
the processor
obtains first current condition information for a user at a first time point of a first period, calculates first dosage form composition information by reflecting the first current condition information in base dose information about a plurality of nutritional components, obtains second current condition information for the user at a second time point of a second period, and calculates second dosage form composition information by reflecting the second current condition information in the base dose information,
the base dose information has a dose value personalized for the user for the plurality of nutritional components, is calculated before the first time point of the first period, and is stored in the memory, and
when calculating the second dosage form composition information, the processor calculates the second dosage form composition information by reflecting the second current condition information in a dose value of the base dose information used when calculating the first dosage form composition information.

9. The electronic device according to claim 8, wherein
the processor calculates the base dose information in advance before the first time point of the first period.

10. The electronic device according to claim 8, wherein
the first or second current condition information is collected from at least one of a device for dispensing dosage forms for the plurality of nutritional components, a user terminal of the user, or an external device for collecting biometric information of the user.

11. The electronic device according to claim 8, wherein
after receiving the first current condition information, the processor calculates first change amount information about a first nutritional component related to the first current condition information among the plurality of nutritional components, and
after receiving the second current condition information, the processor calculates second change amount information for a second nutritional component related to the second current condition information among the plurality of nutritional components.

12. The electronic device according to claim 11, wherein
the processor
when calculating the first dosage form composition information,
for a nutritional component unrelated to the first current condition information among the plurality of nutritional components, calculates base dose information about the nutritional component unrelated to the first current condition information as a daily serving amount in the first period and
for the first nutritional component, calculates the daily serving amount of the first period by including the first change amount information in base dose information about the first nutritional component, and
when calculating the second dosage form composition information,
for nutritional components unrelated to the second current condition information among the plurality of nutritional components, calculates base dose information about a nutritional component unrelated to the second current condition information as a daily serving amount in the second period, and
for the second nutritional component, calculates the daily serving amount in the second period by including the second change amount information in base dose information about the second nutritional component.

13. The electronic device according to claim 8, wherein
the processor
when calculating the first dosage form composition information, calculates the first dosage form composition information by converting a daily serving amount in the first period for the plurality of nutritional components into the number of dosage forms to be dispensed from each cartridge, and
when calculating the second dosage form composition information, calculates the second dosage form composition information by converting a daily serving amount in the second period for the plurality of nutritional components into the number of dosage forms to be dispensed from each cartridge.

14. The electronic device according to claim 8, wherein
the processor transmits the first or second dosage form composition information to a device dispensing the dosage form.

15. The electronic device according to claim 14, wherein
the processor
after the first or second dosage form composition information is transmitted, receives dispensing amount information about the number of dosage forms dispensed from each cartridge of the device from the device, and transmits the dispensing amount information to a user terminal of the user.

16. A dispensing device that dispenses a dosage form comprising:
a memory;
a processor that is connected to the memory and executes instructions stored in the memory;
an input unit that receives information under the control of the processor; and
a dosage form dispensing unit that dispenses a dosage form from a plurality of cartridges storing a plurality of dosage forms for a plurality of nutritional components under the control of the processor, wherein
the processor
obtains first current condition information for a user through the input unit at a first time point of a first period, dispenses the dosage form from the plurality of cartridges through the dosage form dispensing unit according to first dosage form composition information in which the first current condition information is reflected, obtains second current condition information for the user through the input unit at a second time point of a second period, and dispenses the the dosage form from the plurality of cartridges through the dosage form dispensing unit according to second dosage form composition information in which the second current condition information is reflected,
each of the first and second dosage form composition information includes information in which base dose information about the plurality of nutritional components and each of the first and second current condition information are reflected together, and
the base dose information has a dose value personalized for the user and is determined before the first time point of the first period, and the second dosage form composition information is calculated by reflecting the second current condition information in a dose value of the base dose information used when calculating the first dosage form composition information.

17. The dispensing device according to claim 16, wherein
the base dose information is calculated based on personal health information including at least one c of identification information, medical examination-by-interview result information, or medical information for the user.

18. The dispensing device according to claim 16, further comprising:
an output unit that outputs information under the control of the processor, wherein
the processor outputs medical examination-by-interview inquiry information through the output unit before the first time point of the first period, receives answer information from the user through the input unit, and generates medical examination-by-interview result information.

19. The dispensing device according to claim 16, further comprising:
a step of obtaining, by the processor, base dose information for each of the nutritional plurality of components before the first time point of the first period to calculate the base dose information.

20. The dispensing device according to claim 16, wherein
the processor calculates the first dosage form composition information by reflecting the first current condition information in the base dose information, and calculates the second dosage form composition information by reflecting the second current condition information in the base dose information.

21. The dispensing device according to claim 16, wherein
first current condition information is further collected from a user terminal of the user or an external device that collects biometric information of the user until before the first time point during the first period, and
the second current condition information is further collected from the user terminal of the user or the external device that collects biometric information of the user until before the second time point during the second period.

22. The dispensing device according to claim 16, further comprising:
a plurality of dosage form dispensing sensors that detect the dosage form dispensed from each of the plurality of cartridges under the control of the processor; and
a communication unit that transmits and receives information to and from a server under the control of the processor, wherein
the plurality of dosage form dispensing sensors detect a dispensing amount of the dosage form dispensed from each cartridge when the dosage form is dispensed from the plurality of cartridges according to the first or second dosage form composition information to generate dispensing amount information, and
the communication unit transmits the dispensing amount information to the server.

23. A computer program stored in a computer readable storage medium, the computer program
obtains first current condition information for a user at a first time point of a first period,
calculates first dosage form composition information by reflecting the first current condition information in base dose information for a plurality of nutritional components,
obtains second current condition information for the user at a second time point of a second period, and
calculates second dosage form composition information by reflecting the second current condition information in the base dose information,
the base dose information having a dose value personalized for the user for the plurality of nutritional components and being determined before the first time point of the first period, and
the computer program, when calculating the second dosage form composition information, calculates the second dosage form composition information by reflecting the second current condition information in the dose value of the base dose information used when calculating the first dosage form composition information.

24-63. (canceled)
